(19) United States
(12) Patent Application Publication
(10) Pub. No.: US 2025/0166171 A1
(43) Pub. Date: May 22, 2025

(54) METHOD FOR COMPLEX NON-INVASIVE MONITORING OF PHOTONS CAUSED BY THE INTERACTION OF ATOMS WITH ELECTRONS AND PROTONS TO DETECT ORGAN PATHOLOGY AND STRUCTURE DAMAGE

(71) Applicant: Volodymyr Pavlovich Rombakh, Edmonds, WA (US)

(72) Inventor: Volodymyr Pavlovich Rombakh, Edmonds, WA (US)

(21) Appl. No.: 18/513,446

(22) Filed: Nov. 17, 2023

(51) Int. Cl. G06T 7/00; A61B 5/00; G01N 21/88; G06T 7/90

(52) U.S. Cl. CPC G06T 7/0012; A61B 5/0082; G01N 21/88; G06T 7/90

(57) ABSTRACT

The present invention relates to the observation of organic and inorganic processes by measuring radiation emitted by p-photon activity (e.g., p-photon radiation). In at least one embodiment, a method for measuring p-radiation is used to detect structural defects, safely synthesize toxic compounds, and track the growth of organisms inside a human body. Methods and systems are disclosed using electromagnetic radiation, called p-photons, with the help of sensors and artificial intelligence, to read spatial-temporal physical processes in inorganic material, plant cells, and living organs; broadcast information associated with those processes; and store and analyze that information.

### TECHNICAL FIELD

The law of equivalence of mass and energy m_{c}=E/c², formulated by Einstein in 1905, was experimentally confirmed 34 years later during the fission of a neutron-irradiated uranium nucleus. This experiment marked the beginning of the creation of nuclear energy and, in turn, the beginning of nuclear astrophysics. Protons and photons play an important role in the development of astrophysics, being the main carriers of information about processes in the Universe. The paper argues that “the second law of infodynamics supports this principle, potentially validating the idea that information is a physical entity, equivalent to mass and energy.” [See: Melvin M. Vopson, The second law of infodynamics and its implications for the simulated universe hypothesis featured, AIP Advances Volume 13, Issue 10 (2023)]. These conclusions are based on theoretical (mathematical) research, while the mathematical theories of quantum mechanics are based on experimental facts. There is no computer science in the universe, but there are photons that are its carriers.

The proposed method is intended for use in all areas of human activity, including non-invasive detection of organ pathologies at all stages of human life, identification of subcritical damage, or one or more characteristics thereof, in structural elements and devices under nanoscale displacements, including nanoscale displacements of lithospheric layers. The earth before the earthquake. The possibility of practical implementation is predetermined by the properties of photons emitted due to a previously unknown atomic-proton reaction, which occurs during all types of deformations of metal alloys, plastics, glass, wood and natural materials in the temperature range from liquid nitrogen to evaporation of iron; at speed: from the growth of a pine tree to the movement of the Earth and its rotation, as well as from human organs spontaneously in natural conditions. The inventor describes the properties of photons (called p-photons) emitted during an atomic-proton reaction in terms of quantum mechanics and quantum electrodynamics. This conclusion is based on an analysis of 3,765 color photographs obtained from the study of inorganic materials and organs, of which about six hundred were demonstrated. The photons emitted as a result of the atomic-proton reaction are called p-photons, and the resulting photographs are called farograms. The information content of farograms is many times higher than the information content of line and continuous spectrograms caused by atomic-electronic rectification. The creation of computers stimulated the creation of a science called information (in English literature computer science). The improvement of computers, in turn, gave rise to the idea of artificial intelligence and special laws of computer science. Here is a quote from the work [Melvin M. Vopson, The second law of infodynamics and its implications for the simulated universe hypothesis, AIP Advances (2023). DOI: 10.1063/5.0173278]“Dr. Vopson's previous research suggests that information is the fundamental building block of the universe and has physical mass. He even claims that information could be the elusive dark matter that makes up almost a third of the universe, which he calls the mass-energy-information equivalence principle.

The paper argues the second law of infodynamics lends support to this principle, potentially validating the idea that information is a physical entity, equivalent to mass and energy.” These conclusions are based on theoretical (mathematical) research: [Melvin M. Vopson, the second law of infodynamics and its implications for the simulated universe hypothesis featured, AIP Advances Volume 13, Issue 10 (2023)]. Note that the mathematical theories of quantum mechanics are not based on experimental facts. There is no computer science in the universe, but there are photons that are its carriers. The rest mass of photons is zero, but it spreads at the speed of light c it acquires mass mc=E/c2, where E is the photon energy.

The proposed method is based on a natural phenomenon discovered by the inventor based on an experiment called p-radiation (p-photon radiation). He postulates that atomic reactions in the Universe are caused by the interaction of atoms not only with electrons and photons but also with protons. The maximum photon energy in electron-atom interaction occurs when an electric field accelerates the electrons. This radiation is called X-rays. The mass of a proton is 1836 times greater than the mass of an electron, but its charge is positive. This leads to the fact that the properties of the photons produced during this interaction are fundamentally different from those that arise during the collision of an electron. However, this difference is due not only to the proton's mass but also to its charge. The penetrating power of these photons in metal is a million times greater than the power of x-rays and gamma rays, but being born in plants and living organs, they do not damage them.

This apparent paradox is explained by the fact that they are emitted in all directions of space and are not capable of ionizing atoms. Consequently, they are not able to break the bond between atoms. Photon energy is measured with the highest precision and maximum sensitivity. P-photons are easily controlled by optical methods, unlike X-rays. It has been experimentally proven that these p-photons are born during all processes in the Universe. Consequently, the atomic energy of protons proposed in the invention can be used in all spheres of human activity. The creation of a new scientific and technical field called protonics expands the capabilities of atomic energy.

### BACKGROUND

The proposed method uses natural phenomena discovered by the inventor when performing experiments and confirmed by specialists. The properties of photons of this radiation are fundamentally different from those of photons emitted in other regions of the spectrum, regardless of the composition of the object, its size, shape, and nature of external influences. These properties are due to a natural phenomenon called by B. Franklin and M. Faraday, a spark.

Experimental confirmation of Maxwell's electromagnetic theory made it possible to call this phenomenon an electromagnetic impulse. However, experimental studies at the end of the 19^{th} centuries showed that the electromagnetic impulse is insufficient to explain this phenomenon's properties in the Universe since the experiment disproved classical mechanics. This contradiction was eliminated in quantum mechanics and quantum electrodynamics by the recognition that electromagnetic waves exhibit the properties of particles, but also particles, such as an electron, ion, or atom, exhibit wave properties. A particle of an electromagnetic impulse is called a photon. The mathematical theory of quantum mechanics and electrodynamics requires particular study, like any foreign language, but its physical properties are simple and understandable for practical application. This conclusion is confirmed by a laser, a computer, a smartphone, and other devices created based on quantum theories. The method proposed in the invention allows for solving the most important problem of mankind: ensuring the safety of human life based on the early detection of pathologies and viruses, refusing to use X-rays and replacing them with X-rays, preventing man-made disasters based on measuring stored energy, accumulation rate, calculating critical energy and shutting down devices and structures before reaching it; early warning of a possible earthquake or tsunami based on the assessment of energy accumulation by lithospheric plates under nanoscale displacements.

Experimental confirmation of Maxwell's Electromagnetic Theory made it possible to call this phenomenon an electromagnetic impulse. Still, experimental studies at the end of the 19^{th} century showed that the electromagnetic impulse cannot explain this phenomenon's properties in the universe since the experiment refuted classical mechanics. This contradiction was eliminated in quantum mechanics and quantum electrodynamics by recognizing that electromagnetic waves exhibit the properties of particles, but also particles, for example, an electron, an ion, or an atom, exhibit wave properties. A particle of an electromagnetic impulse is called a photon. The mathematical theory of quantum mechanics and electrodynamics requires particular study, like any foreign language, but the physical properties are simple and understandable for practical application. This conclusion is confirmed by the laser, computer, smartphone, and other devices created based on quantum theories. The method proposed in the invention allows for solving the most critical problem of humanity: ensuring the safety of human life based on the early detection of pathologies and viruses, the rejection of the use of X-rays and their replacement with p-rays, prevention of man-made disasters based on the measurement of accumulated energy, accumulation rate, calculation of critical energy and shutdown of devices and structures before it is reached; early warning of a possible earthquake or tsunami based on the assessment of energy accumulation.

The proposed method is intended for use in all areas of human activity, including non-invasive detection of organ pathologies at all stages of a person's life, detection of subcritical damage in structural elements and devices, and nanoscale tissue displacements. Layers of the earth before an earthquake. The possibility of practical implementation is predetermined by the properties of photons emitted due to a previously unknown atomic-proton reaction, which is realized with all types of deformations of metal alloys, plastics, glass, wood, and natural materials.

However, the radiation discovered by the inventor creates regions of a continuous and linear spectrum on a photographic film, sensor, or any light-sensitive material, in a wide frequency range, at which a color farogram is formed on the film. The photographic film can be said to generate one or more colors when reacting to, or interacting with, the radiation comprised of p-photons. The photographic film, or sensor, can also be said to detect the radiation when it reacts to the radiation.

Low refractive and reflective indices hamper the creation of an X-ray microscope. P-rays, having a high penetrating ability, are reflected from the mirror and can be focused, which makes it possible to use p-radiation in nuclear energy, for example, when probing processes in a nuclear reactor with a laser, as follows from the experimental studies of the inventor. However, p-radiation has one more feature: a spatial distribution of frequencies occurs, and a color pattern is formed on the film.

The inventor conducted a total of 446 experimental studies, as a result of which 3,765 farograms were obtained with 100% reliability, of which 1,549 were irradiation of organs. However, only 14% are used for demonstration purposes; the result indicates a natural phenomenon with a strict pattern, which M. Faraday in 1827-1848 pointed out. Particularly interesting are farograms in which luminous and dark areas are repeated many times. One of them turned out to be white fan-shaped white radiation. The inventor discovered the mechanism of this radiation based on Faraday's experiments. Farograms are often observed that have a clear boundary between black and colored areas, accounting for 20% of all used. The colored part of the farogram is due to p-photons. This fact indicates an identical mechanism for forming a solid crystalline cell and a molecule of an organic organ. The appearance of a dark region occurs when atoms do not emit energy but can absorb and store it. However, this does not exclude the possibility that some hydrogen atoms form vacancies, becoming neutral. When a crystal or organ molecule loses a hydrogen atom, it can form a water molecule called water of crystallization.

A living organism contains different types of water. The synthesis of water during the oxidation (combustion) of fats has been known for a long time, but it should be noted that hydrogen is required for this. The experiment confirmed Faraday's predictions and suggested that Rutherford's neutrons form a dark region. The practical significance of this fact can be determined using nuclear magnetic resonance. However, a neutron outside the nucleus decays into a proton, electron, and neutrino, and modern methods should have detected this reaction, but this did not happen. But perhaps there was no problem, and the experiment was not carried out. However, if Rutherford is correct, such a neutron is an iceberg in the Universe with a different lifetime (half-life).

In the sixth lecture, Faraday paid particular attention to the combustion of carbon. The practical significance of this fact can be determined using nuclear magnetic resonance. However, a neutron outside the nucleus decays into a proton, electron, and neutrino, and modern methods should have detected this reaction, but this did not happen. But perhaps there was no problem, and the experiment was not carried out.

It is known that Rutherford gave a lecture at the Royal Society of London in June 1920 [E. Rutherford, Bakerian Lecture: Nuclear Structure of Atoms. Jun. 3, 1920, 370-400]. He proposed that the electron in a hydrogen atom could spin so close to the proton that it would form a neutral doublet (a neutron outside the nucleus) that could move freely through the material.

There are farograms particularly interesting, on which luminous and dark objects are repeatedly repeated. One of these turned out to be white fan-like white radiation. The inventor discovered this radiation's mechanism based on Faraday's experiments. Farograms are often observed, having a clear boundary between black and color areas, making up 20% of all used ones. The colored part of the farogram is due to p-photons. This fact points to an identical mechanism of formation of a solid-state crystalline cell and a molecule of an organic organ. The appearance of a dark region occurs when atoms do not emit energy but can absorb and store it. However, this does not exclude the possibility that some hydrogen atoms form vacancies, becoming neutral.

When a crystal or organ molecule loses a hydrogen atom, it can form a water molecule called water of crystallization. A living organism contains various types of water. The synthesis of water during fat oxidation (combustion) has long been known, but it should be noted that this requires hydrogen. The experiment, which confirmed Faraday's predictions, allows us to conclude that Rutherford's neutrons form the dark region. The practical significance of this fact can be determined with the help of nuclear magnetic resonance. However, the neutron outside the nucleus decays into a proton, an electron, and a neutrino, and this reaction should be detected by modern methods, but this did not happen. But perhaps the problem did not occur, and the experiment was not performed. Faraday, in the sixth lecture, paid particular attention to the combustion of carbon. The practical significance of this fact can be determined with the help of nuclear magnetic resonance. However, the neutron outside the nucleus decays into a proton, an electron, and a neutrino, and this reaction should have been detected by modern methods, but this did not happen. But perhaps the problem did not occur, and the experiment was not performed. Faraday, in the sixth lecture, paid particular attention to the combustion of carbon.

The farograms show a flat, cylindrical border between the dark and colored area and a border of indeterminate shape. Such areas are observed in inorganic (nonorganic) materials and human organs, even when performing a DNA test. The total carbon content in the human body reaches 21%. If we assume that forming such boundaries is due to carbon, then studying this phenomenon is particularly important for medicine, biology, and technology.

Farograms obtained simultaneously in different directions from the source are different, i.e., the figures' frequency, intensity, type, shape, and arrangement are different. For example, farograms of a candle flame obtained from different sides are different. In addition, the inventor conducted experiments in which it was shown that photons emitted by a laser and an LED create p-photons in the material along their entire path.

In this patent application, more than five hundred farograms are given, which show a clear, flat, cylindrical border between dark and colored areas and white-red spherical figures, similar to fireballs, which are conditionally called ball lightning (BL). By decoding farograms, we will undoubtedly create all the prerequisites for creating the quantum theory of strength and destruction and the physics of life that Faraday, Maxwell, and Schrödinger dreamed about.

The proposed method is applicable in all areas of human activity, including medicine, technology, and scientific research. The inventor made this conclusion based on an analysis of experiments set up by M. Faraday Maxwell, the results of which are interpreted based on quantum mechanics and quantum electrodynamics. The inventor repeated several previous experiments, using the photographic method of recording electromagnetic radiation outside the vacuum ultraviolet, without using other methods of its registration.

Experimental studies were carried out using the most straightforward tools; color photographs of photon emission were obtained for all types of deformation of inorganic materials in the temperature range from liquid nitrogen to the evaporation of the iron and in size from the birth of a parasite egg in the inventor's stomach to the sun, from the growth rate of a pine tree to the speed of the Earth's orbit and its rotation, as well as from cells and plant organs in natural conditions. The penetrating power of photons of this radiation in a metal exceeds the penetrating power of X- and gamma rays by a million times. However, they, radiating from the cells of living organs, do not damage them.

Both properties are based on experiments. A paradox has arisen, but one experiment cannot disprove the result of another experiment. Consequently, the paradox is due to an erroneous interpretation of the laws of interaction between atoms, leading to an increase in the bond between them or its rupture. The main error of modern theories of strength is that the strengthening of the bond, in which the synthesis of molecules occurs, the formation of an integral structure or organ, and the weakening, leading to the breaking of the bond and destruction, are due to a change in the structure of the atomic nucleus and its electron shell, but not mechanical impact. This conclusion was reached by M. Faraday and J. C. Maxwell before the creation of quantum theory and R. Feynman when creating quantum electrodynamics. Using the unrealized ideas of his predecessors, the inventor conducted a series of experiments that made it possible to formulate the laws of synthesis and destruction and propose a non-invasive method for detecting the precursors of catastrophic destruction of structures and pathology of organs. Examples and evidence are given in the description of the experiments.

The inventor postulates that the properties of photons and the radiation discovered by him are due to the atomic reaction between an atom and a proton, regardless of the emission of photons caused by the transition of electrons from an upper energy level to a lower one or the exchange of atoms by electrons and photons. The theoretical description of the atomic-proton reaction is given with the help of quantum electrodynamics (QED), which is currently the most accurate physical theory. The difference in the properties of photons in an atomic-proton reaction, called p-photons, from photons in reactions of another type was established based on color photographs, called farograms. The information that can be obtained by reading farograms is necessary to reveal the mechanism of processes both in inorganic materials and in the cells of living organs. M. Faraday demonstrated the synthesis of water during the combustion of wax in a candle flame and iron and zinc in a gunpowder flame in lectures given to young people in 1827. The inventor, repeating experiments with candles, found that a candle flame emits p-photons. In several experiments in the study of inorganic materials and organs, fan-shaped white farograms were demonstrated. A comparison of all the facts led to the conclusion that this radiation is associated with the synthesis of water. Experimental proof of the synthesis of water in inorganic materials, the brain, chest, and other organs is a significant discovery in medicine and technology that should be used immediately.

The properties of photons of this radiation are fundamentally different from those of photons emitted in other regions of the spectrum, regardless of the composition of the object, its size, shape, and the nature of external influences. These properties are due to a natural phenomenon called an electromagnetic pulse, previously called a spark, including by B. Franklin and M. Faraday. The versatility of the proposed method makes it possible to solve the most critical problems of science. It is extending human life and increases its comfort by early detection of pathologies and viruses, using not X-rays but photons emitted by the body, refusing to use electrodes for mapping the brain and heart, improving the quality of medicines and physiotherapy; prevention of man-made disasters based on the measurement of accumulated energy in local groups of metastable atoms (destruction domains), accumulation rate, calculation of critical energy, and shutdown of devices and structures before it is reached; early warning of a possible earthquake or tsunami based on the assessment of energy accumulation by lithospheric plates under nanoscale displacements.

The proposed method is based on the quantum-mechanical interpretation of the experimental results of M. Faraday, his ideas about the magnetic field, and the ideas of J. C. Maxwell, formulated at the birth of the electromagnetic theory. The inventor conducted the experiment recommended by Maxwell, which confirmed Maxwell's hypothesis about the relationship between the mechanical properties of a solid body and optical properties, expressed as early as 1850 in their work “[Maxwell J. C. III. On the Equilibrium of Elastic Solids, pp. 31-74, The Scientific Papers of James Clerk Maxwell, Edited by W. D. NIVEN, M. A., F. R. S. Two Volumes Bound as One. Dover Publication Inc. N.Y], in which he proposed the theory of plasticity of a solid body in equilibrium. The mathematical theory of plasticity under linear deformation, based on two postulates of linear and cubic coefficients of plasticity, is flawless. He gives mathematical solutions to fourteen solid mechanics problems and proposes experimental verification methods. This work should still be an example today, but it is not cited. Maxwell, in a letter to W. Thomson (Lord Kelvin) dated Dec. 18, 1856, proposes an equation in which the energy U=U₁+U₂ predicted by Maxwell is interpreted by the inventor from the standpoint of quantum mechanics as the sum of the binding energy between atoms U₁, and U₂ is the energy of local groups of atoms in a metastable state. In this letter, he proposes a simple equation ΣpdS=0 “where p is the stress perp. to ds. That is the whole pressure=whole tension in the section.” In addition, he repeatedly stated: “strain not to the stresses; strain without stress.” The letter ends with the phrase: “Given the mechanical strain in 3 directions on an element, when will it give way? I think this notion will bear working out into a mathematic theory of plasticity when I have time to compare with experiment when I know the right experiments to make. “This letter, written by Maxwell ten years before the publication of the equations of electrodynamics, became known in 1937. It refutes the modern theories of strength, elasticity, and failure. The experiment he dreamed of is being performed today by a smartphone or computer user. Still, in 76 years, the letter has been cited in scientific papers only thrice without understanding what is written. [See Vladimir Rombakh, Two Interpretations of Maxwell's Solid-State Mechanics, Amazon.com Services LLC (2013), pp. 78].

The possibilities of using the method are due to the properties of the signal, which has a maximum speed equal to the speed of light, maximum penetrating ability, and non-invasiveness in inorganic natural materials, products, plants, and organs, regardless of composition, size, shape, temperature, and speed. The method is applied in a vacuum and any environment. Damage, structural defects, and pathology of organs are detected using farograms obtained remotely from any point in space concerning the source of photons, which is the object under study. The information content of a color and numerical farogram exceeds that obtained by other methods that do not apply to living organs.

The inventor proposes equations for calculating the potential distortion energy based on experimentally determined parameters. Examples of modern experimental methods for identifying and destroying zones for the quantitative assessment of wear, fatigue accumulation (wear rate), critical fatigue (critical wear), residual life, and operating time of safe operation are given. This prevents catastrophic failures. However, the energy of U₂ is due to electron-photon exchange, which is accompanied by p-radiation but not by X-rays, as the inventor previously assumed [See: U.S. Pat. No. 11,047,813 B2].

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-M illustrate farograms related to absorbing mediums, according to at least one embodiment;

FIGS. 2A-H illustrate farograms related to light-emitting diode (LED) radiation, according to at least one embodiment;

FIGS. 3A-F illustrate farograms related to lead plates, according to at least one embodiment;

FIGS. 4A-F illustrate farograms related to lead plates, according to at least one embodiment;

FIG. 5A-F illustrate farograms related to lead plates, according to at least one embodiment;

FIGS. 6A-P illustrate farograms related to a lead plate, according to at least one embodiment;

FIGS. 7A-F illustrate farograms related a rolled lead plate, according to at least one embodiment;

FIGS. 8A-F illustrate farograms related a rolled lead plate, according to at least one embodiment;

FIGS. 9A-F illustrate farograms related a rolled lead plate, according to at least one embodiment;

FIGS. 10A-F illustrate farograms related a rolled lead plate, according to at least one embodiment;

FIGS. 11A-F illustrate farograms related a rolled lead plate, according to at least one embodiment.

FIGS. 12A-F illustrate farograms related a steel beam, according to at least one embodiment;

FIGS. 13A-F illustrate farograms related a steel beam, according to at least one embodiment;

FIGS. 14A-F illustrate farograms related a steel beam, according to at least one embodiment;

FIGS. 15A-F illustrate farograms related a steel beam, according to at least one embodiment;

FIGS. 16A-J illustrate farograms related to a candle flame, according to at least one embodiment;

FIG. 17 illustrate a system to generate a farogram related to a candle flame, according to at least one embodiment;

FIG. 18A and FIG. 18B illustrate farograms related to a candle flame, according to at least one embodiment;

FIGS. 19A-D illustrate farograms related to solar rays, according to at least one embodiment;

FIGS. 20A-O illustrate farograms related to a train rail and/or lead plates, according to at least one embodiment;

FIGS. 21A-P illustrate farograms related to a candle flame, according to at least one embodiment;

FIGS. 22A-C illustrate farograms related to a candle flame, according to at least one embodiment;

FIGS. 23A-C illustrate farograms related to a candle flame, according to at least one embodiment;

FIGS. 24A-L illustrate farograms related to a candle flame, according to at least one embodiment;

FIGS. 25A-H illustrate farograms related to a candle flame, according to at least one embodiment;

FIGS. 26A-H illustrate farograms related to a candle flame, according to at least one embodiment;

FIGS. 27A-K illustrate farograms related to a candle flame, according to at least one embodiment;

FIGS. 28A-Q illustrate farograms related to a hacksaw blade, according to at least one embodiment;

FIGS. 29A-L illustrate farograms related to nail corrosion, according to at least one embodiment;

FIGS. 30A-I illustrate farograms related to sodium hypochlorite in a steel container, according to at least one embodiment;

FIGS. 31A-F illustrate farograms related to sodium hypochlorite in a steel container, according to at least one embodiment;

FIGS. 32A-L illustrate farograms related to sodium hypochlorite in a steel container, according to at least one embodiment;

FIGS. 33A-L illustrate farograms related to steel container and sand, according to at least one embodiment;

FIGS. 34A-L illustrate farograms related to steel container and sand, according to at least one embodiment;

FIGS. 35A-I illustrate farograms related a pipe and water pressure, according to at least one embodiment;

FIGS. 36A-I illustrate farograms related to steel container, sand, and water, according to at least one embodiment;

FIGS. 37A-I illustrate farograms related to steel angle, according to at least one embodiment;

FIGS. 38A-M illustrate farograms related to steel angle, according to at least one embodiment;

FIGS. 39A-H illustrate farograms related to aluminum foil, according to at least one embodiment;

FIGS. 40A-D illustrate results of studies related to X-ray emissions from Earth, concrete, and/or metal, according to at least one embodiment;

FIGS. 41A-H illustrate farograms related to human organs, according to at least one embodiment;

FIGS. 42A-C illustrate farograms related to the human body, according to at least one embodiment;

FIGS. 43A-I illustrate farograms related to the human body, according to at least one embodiment;

FIGS. 44A-M illustrate farograms related to the human body, according to at least one embodiment;

FIG. 45A and FIG. 45B illustrate farograms related to a parasite in the human body, according to at least one embodiment;

FIGS. 46A-E illustrate farograms related to the genesis of organisms, according to at least one embodiment;

FIGS. 47A-I illustrate farograms related to a candle flame, according to at least one embodiment;

FIGS. 48A-F illustrate farograms related to the genesis of organisms, according to at least one embodiment;

FIGS. 49A-I illustrate farograms related to the genesis of organisms, according to at least one embodiment;

FIGS. 50A-I illustrate farograms related to an infrared heating mat, according to at least one embodiment;

FIGS. 51A-H illustrate farograms related to an infrared heating mat, according to at least one embodiment;

FIGS. 52A-H illustrate farograms related to an infrared heating mat, according to at least one embodiment;

FIGS. 53A-I illustrate farograms related to an infrared heating mat, according to at least one embodiment;

FIGS. 54A-K illustrate farograms related to an infrared heating mat, according to at least one embodiment;

FIGS. 55A-K illustrate farograms related to an infrared heating mat, according to at least one embodiment;

FIGS. 56A-C illustrate farograms related to an infrared heating mat, according to at least one embodiment;

FIGS. 57A-I illustrate farograms related to an infrared heating mat, according to at least one embodiment;

FIGS. 58A-D illustrate farograms related to an infrared heating mat, according to at least one embodiment;

FIGS. 59A-G illustrate farograms related to persons of a family, according to at least one embodiment;

FIGS. 60A-H illustrate farograms related to persons of a family, according to at least one embodiment;

FIGS. 61A-L illustrate farograms related to persons of a family, according to at least one embodiment;

FIGS. 62A-F illustrate farograms related to persons of a family, according to at least one embodiment;

FIG. 63A and FIG. 63B illustrate farograms related to persons of a family, according to at least one embodiment;

FIGS. 64A-S illustrate farograms related to persons of a family, according to at least one embodiment;

FIGS. 65A-M illustrate farograms related to the human body, according to at least one embodiment;

FIGS. 66A-F illustrate farograms related to sugar content in the human body, according to at least one embodiment;

FIGS. 67A-O illustrate farograms related to blood of the human body, according to at least one embodiment;

FIG. 68 illustrates at least a portion of a farogram shown in FIG. 67N, according to at least one embodiment;

FIGS. 69A-F illustrate farograms related to urine from the human body, according to at least one embodiment;

FIGS. 70A-F illustrate farograms related to mineral water, according to at least one embodiment;

FIGS. 71A-F illustrate farograms related to an iodine solution, according to at least one embodiment;

FIGS. 72A-G illustrate farograms related to vitamin E oil, according to at least one embodiment;

FIGS. 73A-L illustrate farograms related moisturizing cream, according to at least one embodiment;

FIGS. 74A-H illustrate farograms related omega-3 fish oil, according to at least one embodiment;

FIGS. 75A-I illustrate farograms related to the human body, according to at least one embodiment;

FIGS. 76A-I illustrate farograms related to the human body, according to at least one embodiment;

FIGS. 77A-I illustrate farograms related to the human body, according to at least one embodiment.

FIGS. 78A-E illustrate farograms related to the human body, according to at least one embodiment;

FIGS. 79A-G illustrate farograms related to the human body, according to at least one embodiment;

FIGS. 80A-G illustrate farograms related to the human body, according to at least one embodiment;

FIGS. 81A-N illustrate farograms related to the human body, according to at least one embodiment;

FIGS. 82A-H illustrate farograms related to the human body, according to at least one embodiment;

FIGS. 83A-L illustrate farograms related to the human body, according to at least one embodiment;

FIGS. 84A-D illustrate farograms related to the activities of ants, according to at least one embodiment;

FIG. 85 illustrates a photograph from the fragment of an aircraft, according to at least one embodiment;

FIG. 86A and FIG. 86B illustrate photographs of cracks in silicon, according to at least one embodiment;

FIG. 87 illustrates a table of lost mass related to the corrosion of nails, according to at least one embodiment;

FIG. 88A illustrates a table related to cracks in an aircraft structure, according to at least one embodiment;

FIG. 88B illustrates a photograph of cracks in an aircraft structure, according to at least one embodiment;

FIG. 89 illustrates a chart representing crack energy in an aircraft structure, according to at least one embodiment;

FIG. 90 illustrates a chart representing crack energy in an aircraft structure, according to at least one embodiment;

FIG. 91 illustrates chart representing crack energy in an aircraft structure, according to at least one embodiment;

FIG. 92 illustrates chart representing crack energy in an aircraft structure, according to at least one embodiment; and

FIG. 93 illustrates a block diagram of a computing device that can be used to generate farograms and/or identify aspects of farograms, according to at least one embodiment.

### DETAILED DESCRIPTION

All processes in nature, from forming the simplest ion-molecule of hydrogen, H²⁺, consisting of two protons and one electron, to the cluster of galaxies, are due to atomic reactions in which atoms exchange electrons and photons. An essential feature of an atomic reaction is that no photons are in the atom. The nature of photons is such that they exist only when they move. It is born when the energy of an atom changes and leaves it at the speed of light c, which is the maximum in nature. However, the emission of gamma photons is due to the nuclear reaction in the nucleus. Thus, the experiment demonstrates electromagnetic radiation with high penetrating power, characteristic of a nuclear reaction, but in its absence. Modern quantum mechanics proceeds from the fact that in nature, there are only four types of interatomic interaction: strong (nuclear), taken as 1; electromagnetic, equal to 1/137; weak, equal to 10⁻¹²; gravitational, equal to 10⁻³⁸. A contradiction arose between experiment and theory, but the experiment cannot be refuted. Consequently, there was a need to use quantum electrodynamics.

M. Faraday begins the first lecture with a statement, “I will tell you in five lectures the Chemical History of a Candle. There is no law under which any part of the universe is governed that does not come into play and is touched upon in the chemistry of a candle. There is no better, there is no more open door by which you can enter into the study of science than by considering the physical phenomena of a candle.” [See: The Chemical History of a Candle by Michael Faraday (Author), William Crookes (Editor) 1861, 109 pages].

The inventor concluded that Faraday, starting a series of lectures on the chemical history of the candle, formulated the fundamental law of nature in terms understandable for that level of the physical picture of the world. Quantum mechanics allows us to rephrase it based on the experiments performed by the inventor.

All processes in the Universe are caused by reactions between protons and neutrons in the nucleus and atomic reactions outside it, occurring as a result of the transition of electrons from one energy level to another, the exchange of atoms by electrons and photons; these reactions are accompanied by an atomic-proton reaction that proceeds independently of them, but photons are produced in all types of reactions, the properties of which differ.

Fdf fdf Ignorance of the law regulating a natural phenomenon does not prevent its use. However, knowledge of the law indicates the limit at which the law is not violated. Catastrophic destruction results from disregard for the law. All substances are made of atoms; their interaction is determined by the properties of nuclei, electrons, and photons, which exist only in motion at the speed of light. Quantum mechanics and quantum electrodynamics have become the basis of modern science and technology. However, the theories of strength, plasticity, elasticity, and fracture remain in the position of classical mechanics. Theoretical analysis of experimental results obtained on devices created thanks to quantum mechanics is carried out using erroneous equations. One of the most critical discoveries in quantum theory is using quantum numbers that characterize the state of an object. For an electron in an atom, these are the main quantum, orbital, magnetic, and spin.

Ff Modern experimental equipment with high resolution and sensitivity makes it possible to study the influence of electric and magnetic fields on an individual atom or molecule. Such a study was carried out in the work of [Electric Control of Spin Transitions at the Atomic Scale, Piotr Kot, Maneesha Ismail, Robert Drost, Janis Siebrecht, Haonan Huang, and Christian R. Ast, arXiv:2209.10969 v1 [cond-mat.mes-hall]22 Sep. 2022.]“The measurements were done on TiH molecules that adsorb on the bridge-site between two O atoms of the MgO double layer. They are labelled as TiHOO. Varying the bias voltage continuously, we observe the evolution of the ESR (electron spin resonance spectroscopy) with STM (scanning tunneling microscopy) peak as a function of both bias voltage and external magnetic field at a constant microwave radiation frequency of 61.545 GHz and a microwave amplitude of 20 mV. This is shown for two different setpoint currents of Isp=100 pA and Isp=250 pA.”

Consider the research method (ESR) and sample selection (TiH). The methods of classical mechanics cannot describe the spin of the nucleus of an atom or electron. All descriptions, including the name of the parameter itself, are conditional. However, using this parameter makes it possible to describe the properties of chemical compounds, which is especially important for practical applications.

Titanium-hydrogen compounds have variable valence, in which the number of hydrogen atoms in the molecule varies from two to one, taking fractional values. Fractional valency means the sample simultaneously contains compounds 1 and 2 in a particular ratio. [See: Wang, Xin-Quan; Wang, Jian-Tao (15 Jun. 2010). “Structural stability and hydrogen diffusion in TiHx alloys.” Solid State Communications. 150 (35-36): 1715-1718. doi:10.1016/j.ssc.2010.06.004. Retrieved 10 Mar. 2013] “Structural stability and hydrogen diffusion in TiHx alloys, with x=0-2, have been studied by ab initio total-energy calculations.” But it is necessary to know that spin is a special parameter. It is expressed through Planck's constant. It is expressed in terms of Planck's constant h, or Dirac's constant, equal to Planck's constant divided by ℏ=h/2π. The dimension of which is J·s. This physical parameter is called action. In quantum theory, it is the most important. The spin of particles is a vector quantity and is special since it has only two directions. This summary of one experiment is presented to show, using the ESR method as an example, that modern resonance methods can decipher a farogram since a TiH molecule containing one proton, during atomic-proton reaction captures another proton, and a more stable TiH2 molecule is formed, at which it emits p-photons

TiH+H→TiH2+hvₚ,

where is the frequency of the p-photon. This physical parameter is called action. In quantum theory, it is the most important. The spin of particles is a vector quantity and special since it has only two diametrically opposite directions. Consequently, any method in which the electromagnetic spectrum is studied can be used to obtain farograms. The action of histamine and acetylcholine on the light signal was studied in work [See Xiao, N. et al. A single photoreceptor splits perception and entrainment by cotransmission. Nature https://doi. org/10.1038/s41586-023-06681-6 (2023)]“These two molecules act on different neurons having different functions: one type creates an image, and more synchronizes biological rhythms with a day-night cycle.” The simplicity and reliability of obtaining farograms of inorganic materials, fly larvae, and parasites in the stomach guarantee the successful obtaining of farograms on modern, susceptible, high-precision equipment.

Photonics (manipulation of photons) and nanotechnology (manipulation of atoms to create new compounds and structures using lasers) are used in technology and medicine. The causes of brain pathology are often unknown, and the diseases are hazardous, but invasive research is unacceptable, including using electromagnetic waves. Extrapolation of the results of studies of the mouse brain to the human brain is incorrect despite some interesting results obtained in experimental studies.

A group of researchers from six leading US laboratories [See: Aniruddha Das et al. Large-scale recording of neuronal activity in freely-moving mice at cellular resolution, Nature Communications|(2023) 14:6399 11 pp.] developed a method based on luminescence. They write:

“Here, we introduce a different method to acquire snapshots of single-cell cortical activity maps from freely-moving mice using a calcium sensor called CaMPARI. CaMPARI has a unique property of irreversibly changing its color from green to red inside active neurons when illuminated with 400 nm light. We capitalize on this property to demonstrate cortex-wide activity recording without any head fixation, tethering, or attachment of a miniaturized device to the mouse's head.” However, the method, being invasive, does not apply to the study of the human brain. Experiments performed by the inventor demonstrate intense p-radiation from the brain. The use of this radiation requires only deciphering it.

The properties of photons emitted in an atomic-proton reaction fundamentally differ from those in other reactions. The penetrating power of metal exceeds the penetrating power of X-rays by a million times, but they are emitted from the organs without damaging them. Photons emitted by organs pass through glass and plastic, causing a photochemical reaction. This allows the use of electromagnetic radiation sensors for a given frequency of one or more p-photons. These seemingly contradictory properties are because p-photons are incoherent and multidirectional, and the absorption of an individual photon is not enough to form defects in inorganic materials, but it can be helpful for a living organ. Photonics (manipulation of photons) and nanotechnology (manipulation of atoms to create new compounds and structures using lasers) are used in technology and medicine. The causes of brain pathology are often unknown, and the diseases are hazardous, but invasive research is unacceptable, including using electromagnetic waves. Extrapolation of the results of studies of the mouse brain to the human brain is incorrect despite some interesting results obtained in experimental studies.

A group of researchers from six leading US laboratories [See: Aniruddha Das et al. Large-scale recording of neuronal activity in freely-moving mice at cellular resolution, Nature Communications|(2023) 14:6399 11 pp.] developed a method based on luminescence. They write: “Here, we introduce a different method to acquire snapshots of single-cell cortical activity maps from freely-moving mice using a calcium sensor called CaMPARI. CaMPARI has a unique property of irreversibly changing its color from green to red inside active neurons when illuminated with 400 nm light. We capitalize on this property to demonstrate cortex-wide activity recording without any head fixation, tethering, or attachment of a miniaturized device to the mouse's head.” However, the method, being invasive, does not apply to the study of the human brain. Experiments performed by the inventor demonstrate intense p-radiation from the brain. The use of this radiation requires only deciphering it.

### Analysis of Farograms Based on Visual Research

The inventor uses color photography as the most crucial method of demonstrating the results of an experimental study. A distinctive feature of the proposed method is non-invasiveness, which is especially important in the study of organs, objectivity, one hundred percent reliability, high sensitivity, and ease of registration. The photographic film is placed in a container made of paper that is opaque to visible and ultraviolet rays.

The color and shape of images, called color farograms, are the most important sources of information about proton-electron and atomic-proton reactions, the use of which is the purpose of the present invention. A colored farogram is a record on the plane of electromagnetic radiation of dynamic processes occurring in the volume of the object under study for a certain period. High-frequency p-radiation photons emitted from an atomic-proton reaction cause a photochemical reaction by the Stokes law, i.e., the frequency of the luminescence photon is less than that of the absorbed p-photon. A colored farogram allows you to reveal the mechanism of the process caused by it. For example, white fan-like radiation was observed more than once during the deformation of metals and organs in natural conditions. The inventor concluded that this radiation is due to the synthesis of water, discovered by M. Faraday. Water synthesis plays a massive role in forming cracks and functioning organs, including the human brain. This example shows that replacing a color farogram with black and white is prohibited. The most important information about the processes in inorganic materials and organs is shown on the farogram in the form of linear or solid monochromatic and multi-colored areas, the formation mechanism of which is unclear. However, this does not prevent the possibility of using p-radiation with the help of electromagnetic radiation sensors in this area. Modern experimental equipment and computer programs make it possible to detect and transmit photons at any distance with the maximum speed in the universe, sum up the energy accumulated during the operation of a product, structure, or device, analyze the results obtained, compare them with critical parameters, inform about the technical condition of the structure, device, or degree of organ pathology.

One of the essential features of farograms is the multidirectional p-radiation, which indicates a spontaneous emission. This leads to the fact that two farograms located side by side are different. The spatial distribution of the energy of the p-radiation source reduces the density to such a value that it is not enough to break the bond between atoms. This is due to non-invasiveness, eliminating the loss of solid integrity (porosity, cracking, and fracture) caused by stimulated emission of fracture domain energy, as shown in the inventor's U.S. Pat. No. 11,047,813 B2.

### Note

The inventor uses color photography as the sole method of recording the results of the experimental study. The photo film is placed in a container made of paper that is opaque to visible and ultraviolet rays. (Further: Film). The color and shape of the images, called farogram, are the only sources of information about atomic-proton reactions, the use of which is the purpose of this invention.

Let's call a farogram, a graph, or a set of numbers obtained from the research a farogram. Thus, the first step in the application of the method proposed by the invention is to create a device for creating a farogram, including measurements of the intensity of p-radiation, its frequency, the transmission of information, its storage and use by the object, the safe functioning of which it should provide.

The complex structure of white light was first discovered by I. Newton using dispersion. The polarization of light was discovered at the beginning of the 19^{th} century. J. Fraunhofer observed dark lines in the spectrum of solar radiation. We can undoubtedly note that Faraday observed the Fraunhofer lines in a candle flame. He drew the listeners' attention to the dark area, noting that it was the brightest, but did not explain why.

The discovery of spectral analysis plays a unique role in science and technology. As an exception, spectral lines are observed on the farogram. There is much more information in the farogram than we get based on a line or continuous spectrum due to the transitions of electrons in an atom. But it's encrypted. Decoding is possible only based on a comparative analysis when the same atomic process is studied by another method. A huge role in understanding the properties of light, more precisely electromagnetic radiation, was played by quantum mechanics and quantum electrodynamics, thanks to the prediction of which many discoveries were made. But the invention is a separate sphere of human activity. T. A. Edison, who received 1093 US patents, added: “I did not investigate the laws of nature and did not make significant scientific discoveries. I'm just a professional inventor. All my research and experiments were aimed solely at finding something of practical value.” Edison's recognition means that the user can use the invention without knowing the laws on which it is based. However, the theoretical substantiation of the method is more important than the discovery because it helps to make a discovery, ensuring progress and the creation of new technologies.

Everything indicates that p-radiation confirms the quantum principle of least actions proposed by R. Feynman.

This comparison shows that experimental proof of the existence of a natural phenomenon is sufficient for its use. But quantum electrodynamics revealed the mechanism of some phenomena and predicted them. The mathematical apparatus of quantum electrodynamics is complex, but the physics of the described process is simple. The difference between a farogram and a spectrogram is that it is impossible to repeat it since it is a function of time in the universe but not of the experiment. One of the most critical parameters in physics is action. The dimension of this parameter is the product of energy and time (J·s).

The quantum principle of least action, proposed by R. Feynman, is one of the sections of this theory. There is no doubt that this theory is capable of describing p-radiation, the existence of which has been confirmed by numerous experiments. This will make it possible to decode farograms and offer new methods for analyzing processes in living organs and technical structures.

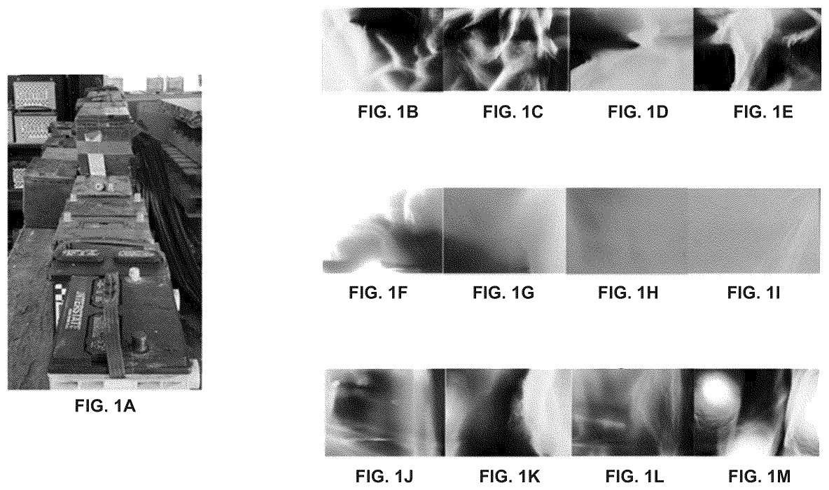

Experiment No. 1, FIGS. 1A-M. The inventor checked Rutherford's idea that a hydrogen atom could form a neutron outside the nucleus. Fourteen lead batteries arranged one behind the other form an absorbing medium 307.34 cm long. The penetrating power of laser radiation was studied using a Bosch Professional GLL laser with a maximum power of ˜1 mW and 635 nm wavelength. The electromagnetic pulse emitted by the laser passed almost unhindered through 307.34 centimeters of lead-acid battery filler. No signs of the appearance of a neutron were found. Consequently, such a high penetrating ability of an electromagnetic pulse is due to another natural phenomenon in which the propagation of an electromagnetic pulse occurs with virtually no energy loss. FIGS. 1B-E show results using an absorbing medium that was 124.46 cm long. FIGS. 1F-I show results using an absorbing medium that was 106.68 cm long. FIGS. 1J-M show results using an absorb in 7-10 g medium that was 76.2 cm long.

Experiment No. 1, Farograms shown in FIGS. 1J-M are obtained by passing the pulse of the battery section at the end. The brightness of the figures does not differ from those of the figures a-d obtained after passing the first section. It means that the pulse energy has practically not changed. The experiment confirmed the pulse's high penetrating power, but the farogram formation mechanism remained unrevealed. It should be noted here that the laser was at a distance of one meter from the first battery, and between the thirteenth and fourteenth batteries, there was a gap of 60 cm. Let's focus on the farogram shown in FIG. 1M, which resembles ball lightning (BL). The bright white central region indicates the presence of a p-radiation source with high energy luminosity in a wide spectral range, causing a white glow. The frequent observation of such a farogram is due to the law, the disclosure of which will help to understand the mechanism of a hazardous type of corrosion called pitting.

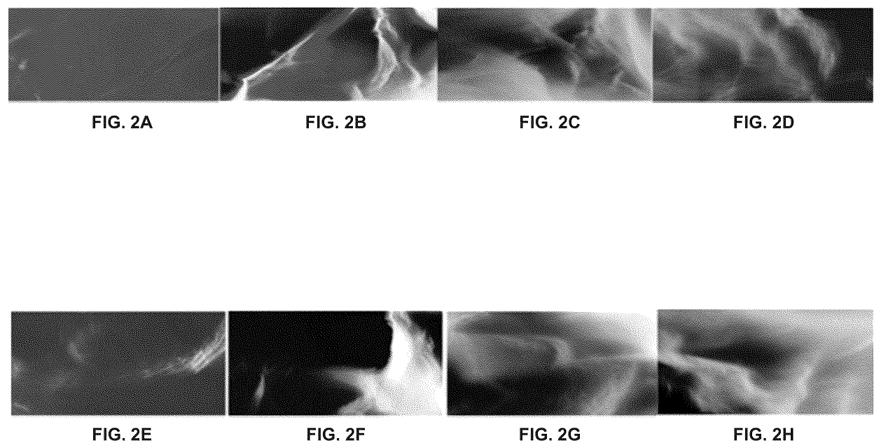

Experiment No. 2, FIGS. 2A-D. The farograms shown in the frames FIGS. 2A-D illustrate the penetrating power of the LED radiation passing through a lead battery, the length of which was 16 cm.

Experiment No. 3, FIGS. 2E-H The farograms shown in the frames FIGS. 2E-H illustrate the penetrating power of the LED radiation passing through a lead battery, the length of which was 28 cm.

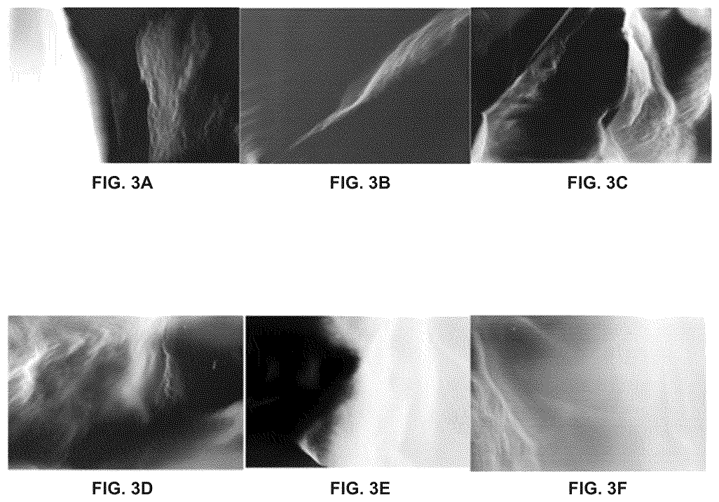

Experiment No. 4, FIGS. 3A-F. Two lead plates 0.12 cm thick and 30 cm long each were irradiated with a laser beam over 600 s. The Film was placed under the plates.

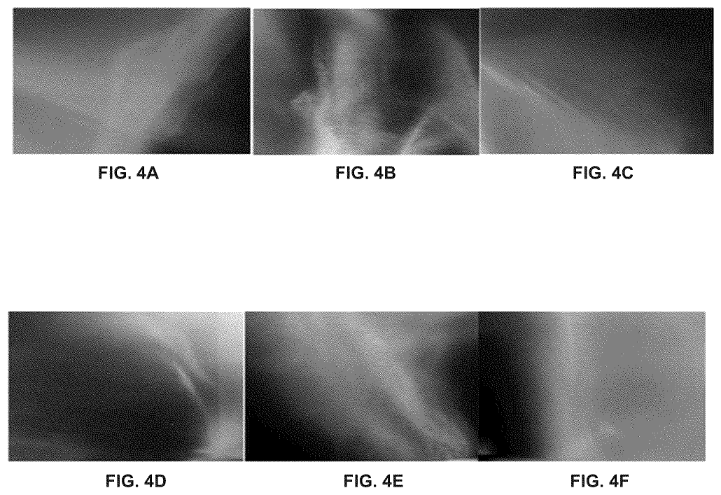

Experiment No. 5, FIGS. 4A-F. Four lead plates 0.12 cm thick and 30 cm long each were radiated with a laser beam over the entire length for 60 s. The Film was placed under the plates.

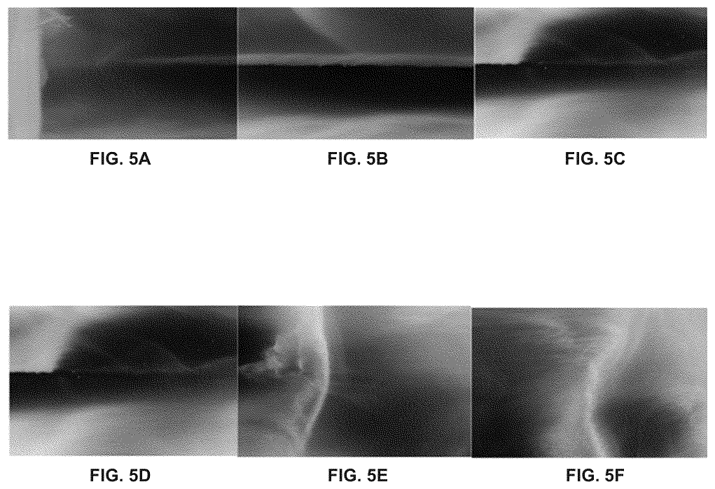

Experiment No. 6, FIGS. 5A-F. Seven lead plates, which were 0.12 cm thick and 30 cm long, were irradiated with a laser beam over the entire length for 12 s. The Film was placed under the plates.

Experiments 4, 5, and 6 were performed so that each subsequent exposure decreased, but the thickness increased. It assumed that the energy absorbed by the photo emulsion would decrease in the ratio of 1:0.05:0.0057, which only leads to a decrease in the brightness of farograms. The brightness of the farograms decreased, but there was a change in the spectral composition and shape of the luminous objects. The wavelength of the laser is longer than the wavelength of the objects shown in the farograms, especially in FIGS. 3A-F. It is contrary to the Stokes law. Still, it can be justified by doubling the laser frequency. The alternation of bands, characteristic of wave interference, is observed in most farograms.

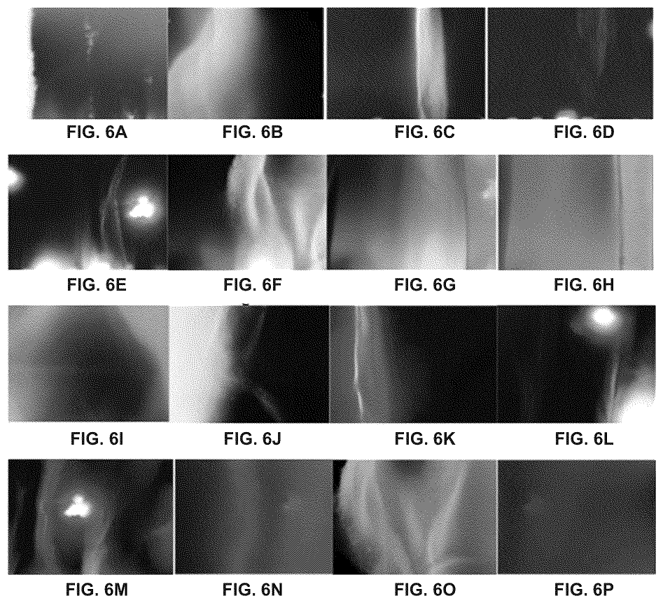

Experiment No. 5, FIGS. 6A-P. A 3 mm thick and 30 cm long lead plate was irradiated with LED radiation for 4 minutes. Farograms obtained on the Film under it are shown in frames shown in FIGS. 6A-H. The plate was irradiated with laser radiation for 4 minutes with a photo film placed under it. Farograms are shown in frames shown in FIGS. 6I-P. It is necessary to pay attention to the similarities and differences of the formed figures.

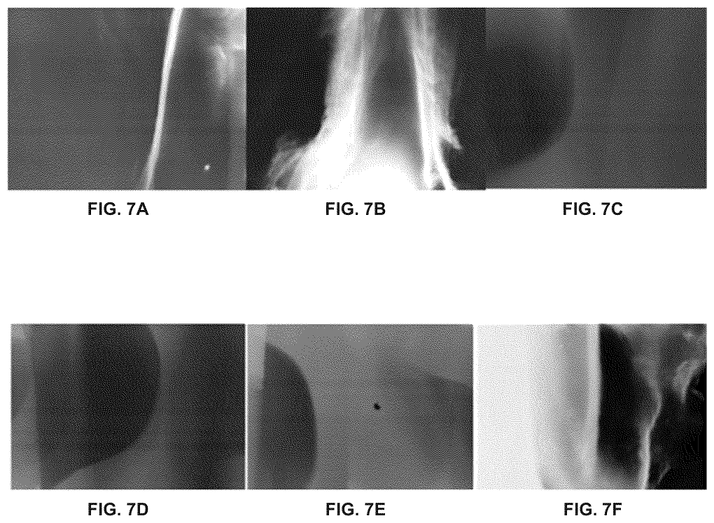

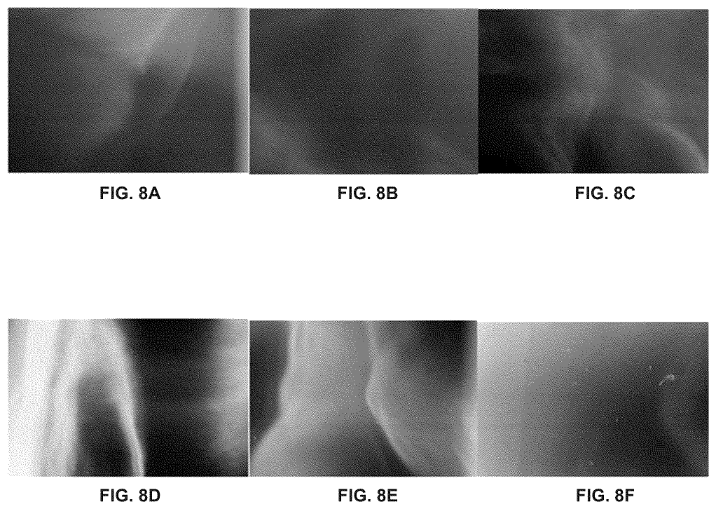

Experiment No. 6, FIGS. 7A-F and FIGS. 8A-F. A lead plate 30×30 cm², 1.2 mm thick, was rolled into a roll consisting of six layers, the maximum diameter of which was equal to the width of the Film. It was located in a west-east direction. Thus, the Films recorded the radiation caused by the laser photons directed to the north (See FIGS. 7A-F) and south (See FIGS. 8A-F).

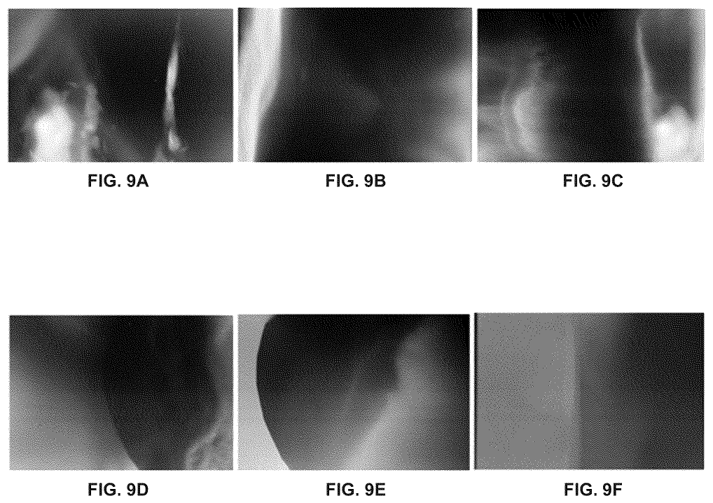

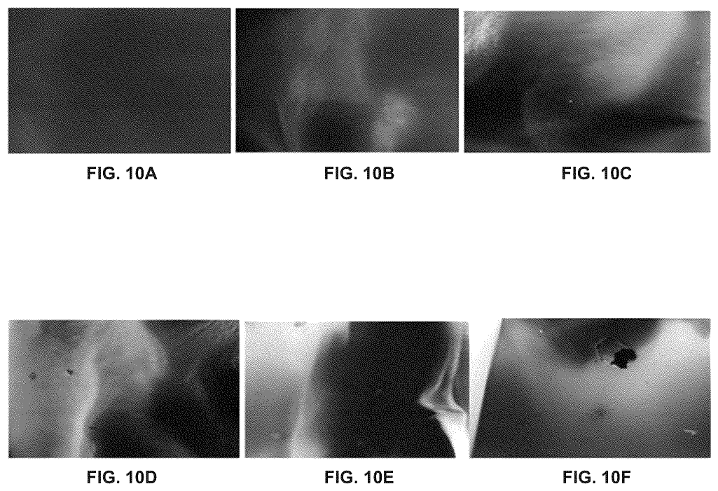

Experiment No. 7, FIGS. 9A-F and FIGS. 10A-F. The experiment was carried out similarly to the previous one but differed in that the roll was located north-south, and farograms were obtained on the east (See FIGS. 9A-F) and west side (See FIGS. 10A-F).

FIGS. 7A-F, FIGS. 8A-F, FIGS. 9A-F, and FIGS. 10A-F demonstrate farograms received from all four cardinal points.

Experiments No. 6 and No. 7 were performed to study the total influence of the magnetic field of the Earth and the Sun, the influence of the forces of attraction of the Sun, the Moon, and the Coriolis force. The farograms obtained on each of the cardinal points are different. A similar result was obtained when a candle was located in the center of the square. Three experiments are insufficient to determine how much this effect can be used for navigation. Note that the parabolic boundary is observed on six farograms, which is 25%. If we consider two more flat borders separating the black area from the color one, we should note that there is a law by which such a phenomenon is due. Such an effect is observed during the deformation of a solid, radiation from organs, and, as we see, during the movement of the Earth.

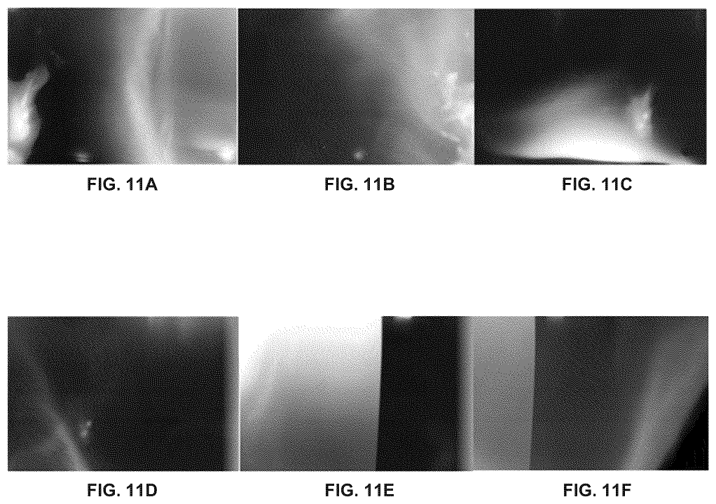

Experiment No. 8, FIGS. 11A-F. Permanent magnets were located along the side surface of a lead roll, the upper surface of which was irradiated with a laser beam. The farograms were obtained on a Film along the roll surface's bottom.

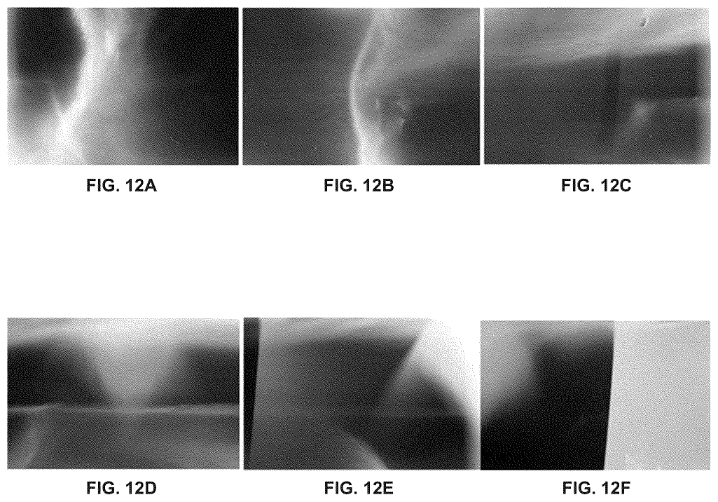

Experiment No. 9, FIGS. 12A-F. Permanent magnets were located along the side surface of a steel beam, the upper surface of which was irradiated with a laser beam. Farograms were obtained on a Film located along the lower surface of the roll.

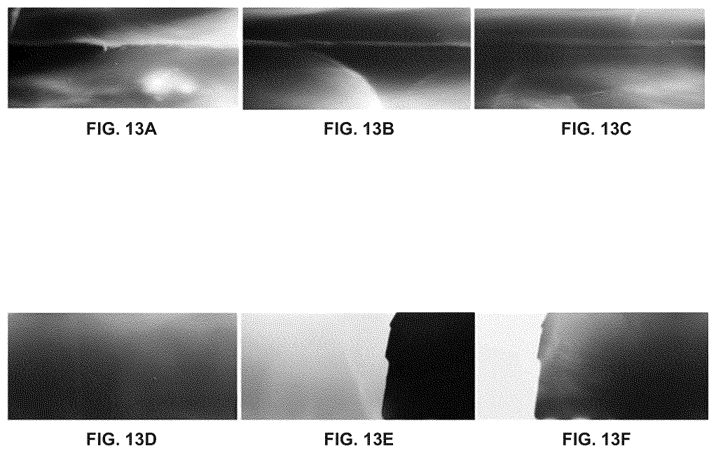

Experiment No. 10, FIGS. 13A-F. A steel beam, having the shape of a rectangular parallel piped 8×8×30 cm3, was located in the north-south direction, while the 8×8 cm² surface was facing north. This surface is irradiated with a laser beam as a thin line. The Film was placed on the surface facing east. The permanent magnets were placed on a surface facing west. The exposure time was 11 seconds. We should note that the ray trajectory is fixed on the farograms, shown in FIGS. 12A-F and FIGS. 13A-F.

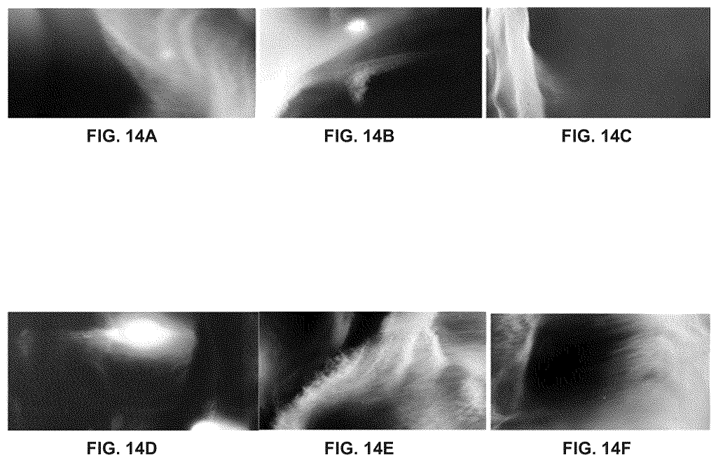

Experiment No. 11, FIGS. 14A-F. This experiment was carried out similarly to the previous one, but the upper surface was a blow at the exposition time.

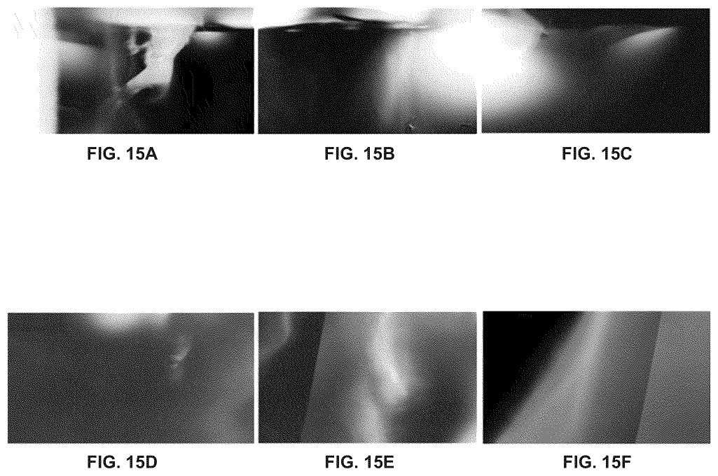

Experiment No. 12, FIGS. 15A-F differed from the previous one in that the beam was located west-east, and the Film was placed on the surface facing north.

The inventor carried out an experimental study to understand to what extent the method proposed by him is consistent with the prediction of E. Rutherford, despite the generally accepted opinion that the transformation of a proton into a neutron occurs only in a nuclear reaction.

So, the farograms shown in FIGS. 1A-M, FIGS. 2A-H, FIGS. 3A-F, FIGS. 4A-F, FIGS. 5A-F, FIGS. 6A-P, FIGS. 7A-F, FIGS. 8A-F, FIGS. 9A-F, FIGS. 10A-F, and FIGS. 11A-F demonstrate the high penetrating power of the photons emitted by the laser and the photodiode. Furthermore, they confirm the inventor's hypothesis that p-radiation is electromagnetic since the magnetic field did not affect the rectilinear propagation of the signal, as depicted in farograms shown in FIGS. 5A-D, FIG. 12D, FIG. 12E, and FIGS. 13A-C. The absence of a charge in photons has been experimentally proven with an accuracy of up to 33 decimal places. A magnetic field does not act on a photon. But the impact resulted in a change in the farograms, in which the glow caused by the laser radiation disappeared, as shown in FIGS. 14A-F and FIGS. 15A-F. It is observed in nine farograms out of a hundred.

It means that other processes leading to high transparency are realized in nature. Note that this conclusion is doubtful and requires additional evidence.

Another energy source is the sun, without which plant growth and life are impossible. The third source of energy that causes p-radiation is gravity. The influence of gravitation on the processes between atoms on Earth is so tiny compared to nuclear and electronic ones that it is neglected. But cannot ignore the gravitational pull of the sun and moon. Only gravity can explain the difference between the farograms shown in FIGS. 7A-F. Farograms obtained from the study of electromagnetic radiation of a laser and LEDs expand the possibilities of the method proposed in the inventor's patent U.S. Pat. No. 11,047,813 B2. For example, the high penetrating power of the p-radiation of a laser photon makes it possible to use it in metallurgy to control processes in the blast and open-hearth furnaces, integrity assessment of multi-ton ingots, search for damage in nuclear reactors.

The role of the magnetic field in nature and technology is enormous. The beneficial effect of a magnetic field on an organ and the detrimental effect of magnetic storms on it are well known, but the photon is neutral and does not interact with it. The charge of a neutron is also equal to zero, but unlike a photon, it interacts with an external magnetic field due to a magnetic moment. Using farograms allows you to create new magnets and understand the mechanism of influence of the magnetic field and magnetic storms. The ease of obtaining farograms and their information content predetermines the need for their use.

### M. Faraday's Candle and P-Radiation

M. Faraday performed and recorded more than 30,000 experiments, some of which he demonstrated at lectures for young people, which are of great scientific importance due to his discoveries. These lectures, first published in 1861, were republished in all developed countries but are still ignored in the scientific and technical literature. They are unknown to students at technical universities. The inventor concluded that the processes occurring in the flame of a candle and gunpowder are accompanied by p-radiation. Some of them are reproduced by him and are shown in this application.

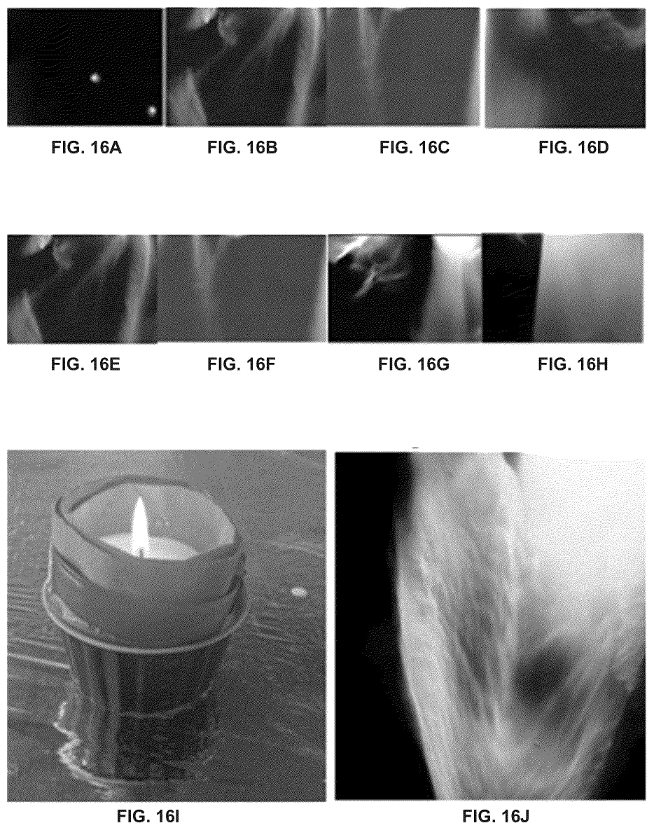

Experiment No. 13, FIGS. 16A-J. The experiment was conducted in a dark room at night for 30 minutes. Frame b, shown in FIG. 16B, demonstrates the flame of a candle in the p-rays. An experiment to test the hypothesis that the radiation of a candle, which M. Faraday demonstrated in his lectures, should be accompanied by p-radiation, was carried out by the inventor using seven thin burning candles located on a container with photographic film. Photos a-h, shown in FIGS. 16A-H, illustrating p-radiation of melting paraffin, confirm the hypothesis. Frame A, shown in FIG. 16A, was obtained when the first candle burned out completely. The rest of the candles were extinguished until they were completely burned out. It illustrates two red and white objects observed for both inorganic and organic objects. Let's call them conditionally the lighting ball (LB). The mechanism of LB formation is unknown.

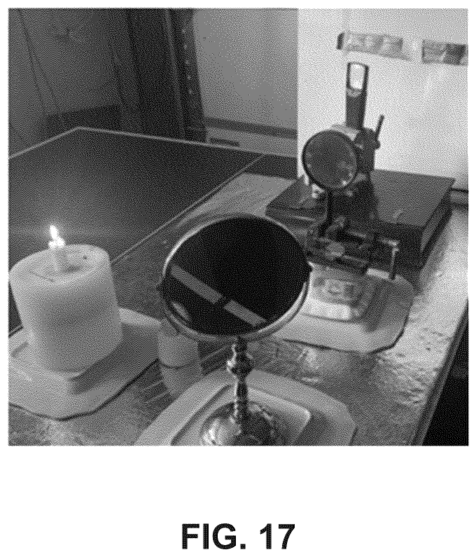

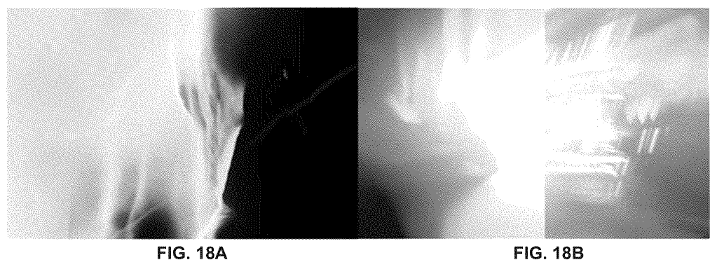

Experiment No. 14, FIG. 17. The farograms demonstrate an experimental study of the possibility of interference of p-radiation when combining an image obtained with two lenses. Light from a candle passes in two directions: through a rectangular lens and, reflected from a mirror, through a round one. The Film is placed on the screen. Spots formed by different beams on the screen are shown separately for demonstration. During the experiment, they are combined. The result of the experiment is shown in FIG. 18A and FIG. 18B. In a rectangular area, interference fringes are observed. The mechanism for the formation of the red line is unknown.

Experiment No. 15, FIG. 18A and FIG. 18B. The farograms show the result of the second experiment to observe interference, performed with one lens. Mirror, candle, lens, and screen all lined up. Thus, there has been interference between beams emitted by the same atom since photons emitted by different atoms are not coherent.

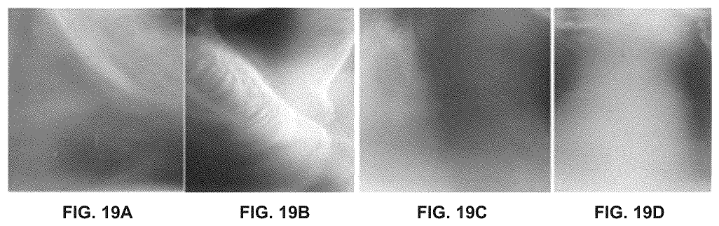

Experiment No. 16, FIGS. 19A-D. Farograms shown in FIGS. 19A-D were obtained by irradiating with a Film with solar rays passing through the glass of a window. The interference pattern observed in frame b is due to the window glass or a thin opaque film inside the container. Still, the very fact of observing the interference of p-rays emitted by the Sun on Earth is significant.

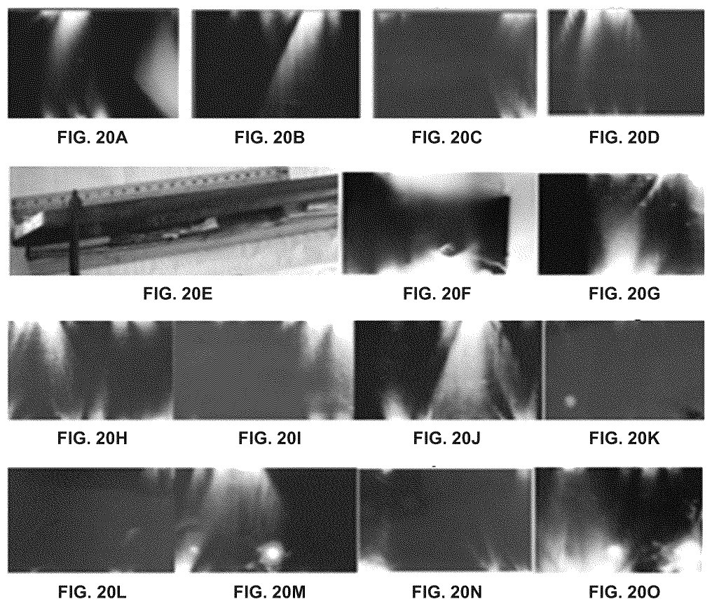

Experiments No. 17, 18, 19; FIGS. 20A-O. Farograms shown in FIGS. 20A-O are obtained from three experiments, two performed using a driving locomotive. Films were located on different surfaces of the rail. The locomotive approached them, moved away, slowed down, and accelerated. The maximum distance at which farograms were taken was 800 m. The first study was carried out in winter. Snow lay on the rails. The farograms shown in frames a and b were ˜10%. It has been hypothesized that they are due to the deformation of snow or ice. But when re-examined in the summer, their number did not change. Two farograms are shown in frames c and d, FIG. 20C and FIG. 20D. An experiment was performed on the device shown in frame e, FIG. 20E. The Film is placed on a wooden bar. Six stacks of lead plates with a thickness of up to 15 mm were stacked. The blow is applied to a steel beam with a section of 38×38 mm² located on lead plates. Ten farograms illustrate fan-like radiation at the film boundary. If we assume that this is water, then the question arises about its source in metals. Faraday answered it.

M. Faraday's ingenious foresight is that he, not knowing about the proton, predicted the unique role of hydrogen in those processes that we now call exothermic and endothermic atomic reactions. He, using simple experiments, proved that when iron and zinc burn, hydrogen is released, which, reacting with atmospheric oxygen, forms water. Attempts to eliminate hydrogen in vessels using the most advanced modern vacuum creation methods are unsuccessful because protons freely penetrate the vessels' walls. This means that, by violating the integrity of the material, we contribute to the formation of water, the monoatomic layer of which becomes one of the reasons for the formation of cracks. The synthesis of water is an exothermic chain reaction, as a result of which the emitted photons are incoherent and multidirectional. Such partially absorbed photons heat a solid or liquid but cannot break the atoms' bond. The high penetrating power of p-photons leads to the fact that the photons have left the place where the synthesis is made. The chemical reaction of water synthesis is no different from those reactions called corrosion. Therefore, another energy source is needed for pore formation, crack formation, and fracture. The modern theory of strength considers mechanical stress to be such a source, neglecting that Newton's force cannot be applied to the atom since the wave properties of electrons and ions prevail at a distance of 4 nanometers. The electron microscope, field ion microscope, and neutron diffraction are based on the wave properties of particles.

All this indicates that the energy source sufficient for destruction is a local group of metastable (long-lived) atoms. Such atoms have been observed many times in experiments conducted by the inventor. P-radiation from metal particles formed during destruction was delayed by a thousandth of a second. This means that a second energy source is a local group of metastable atoms, called a destruction domain, that emits spontaneously and stimulated energy, like a laser.

Stimulated photons are coherent and emitted in one direction; their energies are summed up. Only such photons create pores and cracks and lead to destruction.

A pore or crack formation is possible only when the bond between a certain number of atoms is broken. The bond is broken only when the repulsive force rises sharply and exceeds the force of attraction. A change in dynamic equilibrium at a certain bond length occurs due to a change in the electron shell of atoms caused by the impact of an electron, ion, or absorption of a photon.

P-photons emitted during the synthesis of water form a fan-shaped figure. P-photons emitted during a chemical may form a different shape indicative of that chemical reaction. Such figures have been repeatedly observed on inorganic materials, plant cells, and organs of living organisms. The experiments of M. Faraday demonstrated combustion processes in which hydrogen is released, water is formed, and carbon dioxide is formed. The connection of these processes with human life is essential for biology and medicine. Still, scientific publications in which M. Faraday's lectures would be quoted are not unknown to the inventor. The reproduction of these experiments by the inventor showed that each experiment is accompanied by p-radiation. The quantum mechanical interpretation makes it possible to use this radiation to control the creation of materials and products during operation to prevent failure leading to disaster.

The synthesis of water is a source of energy for the vital activity of organisms. It plays a unique role in the brain's functioning, which consumes 20% of a person's energy. The calorific value of hydrogen (the energy released during combustion) is 141 megajoules/kilogram, which is the maximum for elements and compounds from them. Ignition of a mixture of hydrogen and oxygen (chain reaction) begins with the absorption of two photons that ionize hydrogen atoms. The device for testing the oxygen content in the blood plays a vital role in treating diseases. Undoubtedly, the control of water synthesis, which is easy to implement, will be used. The simplicity of detecting p-radiation, one hundred percent reliability, versatility, and low cost allow it to control processes in all branches of human activity.

The emission of a photon accompanies each exothermic atomic reaction, the parameters of which are the frequency v related to the wavelength λ by the

$$c = λ v ) , \qquad ( 1 )$$

where c is the speed of electromagnetic waves in the medium; energy

$${ε}_{p} = hv , \qquad ( 2 )$$

where h is Planck's constant. The emission of a photon is associated with the transition of an electron from the upper-level E₂ to the level E₁ located below. Let us pay attention to the fact that the dimension of the Planck constant J·s, i.e., the dimension of action.

Photon energy

$$hv = {E}_{2} - {E}_{1} . \qquad ( 3 ) .$$

The charge of a photon and its rest mass are equal to zero.

A. Einstein theoretically showed that the mass of bodies is not constant and depends on speed, contrary to Newton's postulate. He concluded that the energy of a body and its mass are related by the equation

$$E = {mc}^{2} . \qquad ( 4 )$$

This means that the photon has mass but only in motion. The mechanism of electromagnetic radiation of atoms is revealed with the help of quantum mechanics and quantum electrodynamics.

The successes of modern technology, biology, and medicine are associated with the development of quantum theory. Equation (4) has been experimentally confirmed and is currently the basis of nuclear energy. But in the works devoted to the technology of materials, their strength, and destruction, the achievements of quantum theory have been ignored for a hundred years.

P-photons are invisible but cause luminescence, making it possible to control processes visually and with the help of instruments that determine the parameters of emitted photons.

The inventor, using p-radiation, studied the processes from the synthesis of inorganic compounds to the appearance of fly larvae in rotting fish and chicken eggs, parasites in his stomach to the functioning of the brain; the process in inorganic objects from the temperature of liquid nitrogen to the melting and evaporation of the iron; synthesis of compounds and their destruction. To paraphrase M. Faraday, the inventor has the right to assert that no such process in the Universe would occur without p-radiation. This allows him to rephrase the fundamental law of nature that Faraday spoke about when he began his cycle of lectures on the candle flame.

The inventor, interpreting the law discovered by Faraday, relied on the work of A. Zewail on femtochemistry, especially in the part in which femtochemistry confirmed the predictions of L. Pauling set out in the book [Linus Pauling, The Nature of Chemical Bond, 3 Edition, 1960]. The inventor, interpreting the law discovered by Faraday, relied on the work of A. Zewail on femtochemistry, especially in the part in which femtochemistry confirmed the predictions of L. Pauling set out in the book [See Linus Pauling, The Nature of Chemical Bond, 3rd edition 1960] and the rationale Belousov-Zhabotinsky reaction. We only note that p-radiation was discovered by the inventor based on simple experiments like Belousov discovering the reaction.

R. Feynman, proposing a new area known as the nano-region, noted that other laws operate in it than at the top. This prediction came true. New effects were discovered, for example, the quantum mirage and the quantum Hall effect. Undoubtedly, the atomic-proton reaction can be investigated by all those methods that demonstrate high efficiency. This will make it possible to decode farograms and propose new methods of using them to create a new branch of science, technology, and medicine, which can be called photonics.

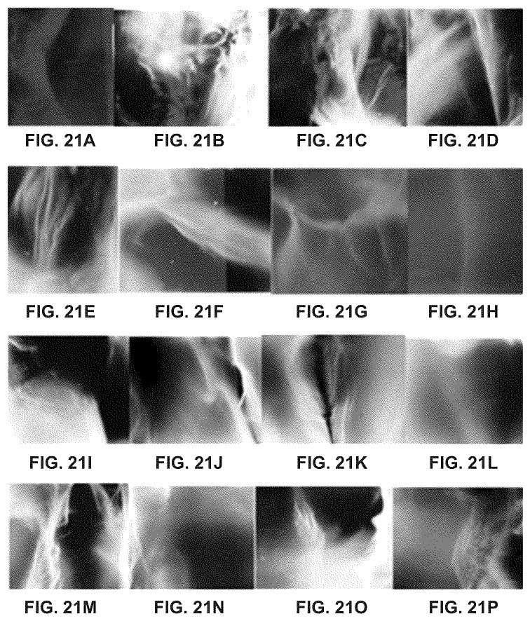

Experiment No. 20, FIGS. 21A-P. Two Films were placed around the candle flame, as shown in FIGS. 16A-J, but one Film over another. The frames shown in FIGS. 21A-H illustrate the farograms obtained on the upper Film; The frames shown in FIGS. 21I-P illustrate farograms obtained on the lower film. The analysis illustrates their similarities and differences. Even the farograms of the wick, shown in FIG. 21J and FIG. 21K, obtained after ˜15°, are noticeably different. Faraday drew the listeners' attention to the dark area near the wick, stating that it was the brightest, but did not explain why. There is no doubt that Faraday knew about the Fraunhofer lines in the sun, but he demonstrated them when irradiated with an electric lamp. We can only assume what Faraday saw, believing that all the laws of nature are manifested in the flame of a candle. Spectral analysis, self-reversal of spectral lines, and resonance have not been proposed. It is known that P. Zeeman discovered in 1896 that spectral lines split in a magnetic field, but noted that the idea belongs to M. Faraday.

An analysis of the farogram of fan radiation showed that it is due to the synthesis of water. This, in turn, helped reveal the mechanism of hydrogen embrittlement and crack formation. This example demonstrates the importance of correctly interpreting farogram objects. The farogram of FIG. 21G shows a clearly defined boundary between the black and color regions crossed by white radiation. The black area has been repeatedly observed in inorganic and organic materials, including organs. The mechanism of the formation of such areas by the inventor is not disclosed and remains mysterious. The experiment showed that all the processes inherent in light radiation manifest in p-radiation. The repeated repetition of farograms testifies to a mechanism by which they are conditioned. Of particular importance is the disclosure of the mechanism of the farogram.

Faraday used the flames as a detector. The inventor repeated Faraday's experiments, irradiating the flame of a candle with an electric lamp, a voltaic arc, a laser, an LED, a router, and radiation from the hands and head of a person.

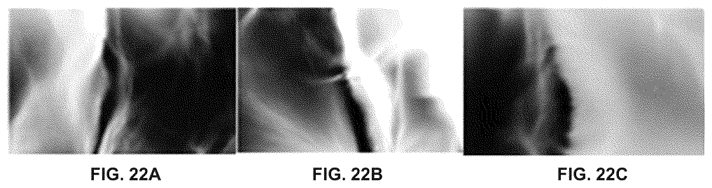

Experiment No. 21, FIGS. 22A-C. Farograms shown in FIG. 22A and FIG. 22B illustrate photographs of a candle flame when irradiated with light from a voltaic arc. In this case, the farogram shown in FIG. 22C is obtained by irradiation with electromagnetic radiation from the router. Note that the energy of a photon of ultraviolet radiation of a voltaic arc exceeds the energy of photons of a router by 5-6 orders of magnitude. However, in both cases, a dark region is observed near the wick, indicating that the atoms of the flame absorb photons emitted by an external source.

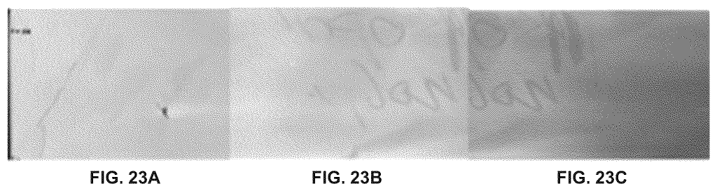

Experiment No. 22, FIGS. 23A-C. Three farograms out of 12, obtained by irradiating a candle flame with electromagnetic radiation from a router, show the latent JaNJaN of the inscription on a paper container, opaque to visible and ultraviolet rays, is imprinted on photographic film. Therefore, p-radiation can be used in archeology, forensics, and restoration of paintings or inscriptions.

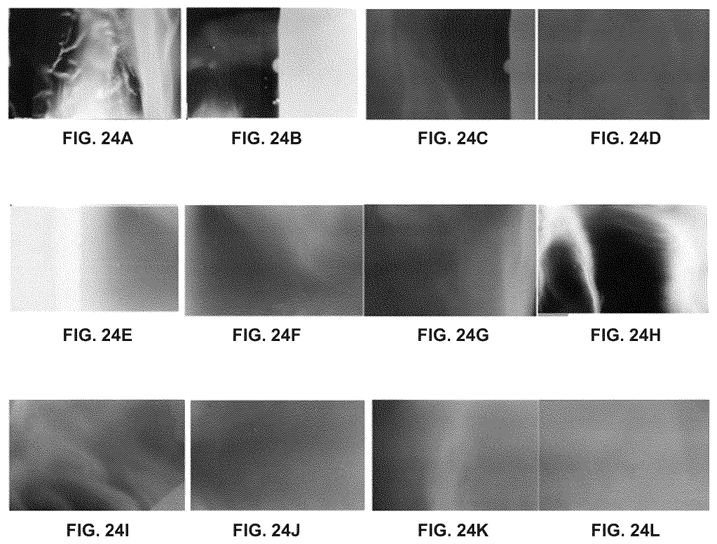

Experiment No. 23, FIGS. 24A-D. The flame of a candle was focused on the screen using a lens. It was illuminated by LED radiation perpendicular to the candle-screen direction. This ruled out the possibility of a direct hit on the screen by p-photons emitted by a photodiode. Farograms shown in FIGS. 24A-D illustrate the result of the action of photons of the p-radiation of the LED on the p-radiation of the candle.

Experiment No. 24, FIGS. 24E-L. The farogram of a candle was focused on the screen using a lens. It was illuminated by laser radiation perpendicular to the candle-screen direction. This ruled out the possibility of p-photons emitted by the laser hitting the screen directly. Farograms shown in FIGS. 24E-L illustrate the result of the action of photons of the p-radiation of the laser on the p-radiation of the candle.

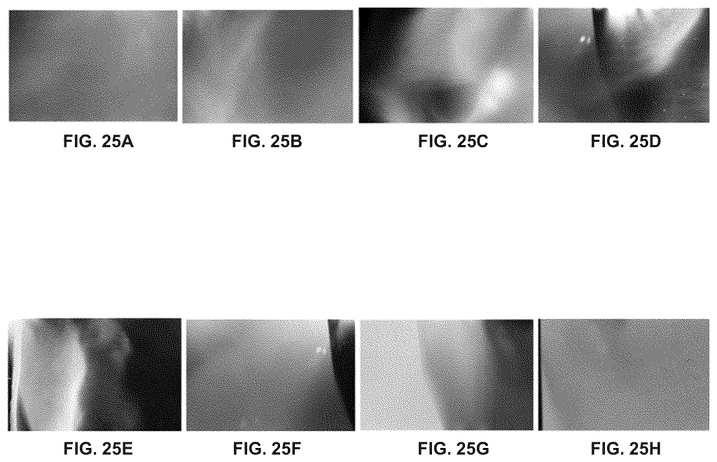

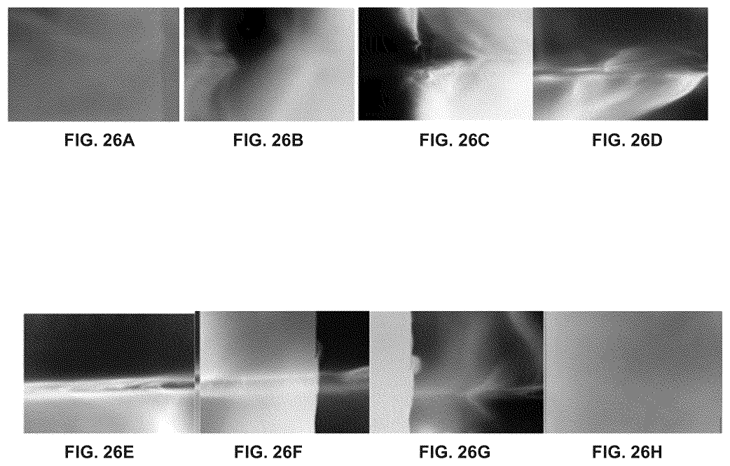

Experiment No. 25, FIGS. 25A-H. The experiment was carried out similarly to experiment no. 24, but the photodiode's emission direction coincided with the candle's emission direction. Farograms shown in FIGS. 25A-H illustrate the effect of photons from the p-radiation of the LED on the p-radiation of the candle.

Experiments No. 24-26 were performed to evaluate the effect of radiation, both the laser and the LED, on the p-radiation of the candle. Analysis of the research results shows that a thin layer of candle flame selectively absorbed laser radiation.

The presence of a border between dark and colored areas, observed repeatedly, indicates the existence of a law that determines this. Revealing the law that determines this is particularly important for understanding the role of each chemical element in an alloy, composite, and organ, including the proton. It is present in the material even when it is undesirable. Farogram provides the most accurate and objective information about the process by which it is due, but this information is encrypted. An example of water synthesis shows that the shape of a luminous and dark object allows us to understand the mechanism of the process, and the spectral composition informs those chemical elements involved in the process, which may provide one or more indications of one or more characteristics of damage to an object. Farograms were obtained due to the absorption of photons by photographic material. The disclosure of each farogram's formation mechanism is of particular importance.

Experiment No. 27. The Film was placed on a stone embankment of a railway track four meters from it, along which a train consisting of 113 loaded platforms passed. The Geiger counter was located at a distance of 0.5 m from the rails. Twelve farograms were received, but the radioactive background practically did not change. This fact testifies to the safety of p-radiation but requires explanation.

The experiment shows that any process in the universe is caused by an atomic or nuclear reaction, which is accompanied by the emission of photons. Neglect of this fact is unacceptable, as it leads to disaster. But until now, theoretical materials science has ignored this fact. The title of many works on materials science begins with the words “Atomic mechanism . . . ” but the description of the atomic mechanism is possible only with the help of quantum mechanics. However, the authors of these publications replace it with mathematical models and equations with no physical meaning. For example, in [M. Chang, Y. Zang, L. Mang, et al. The atomic mechanism of notch sensitivity of the deformation mode in metallic glasses, J. Appl. Phys. 131, 225108 (2022), 1-9]. The authors used stress-strain curves and atomic Voronoi volume.

The surface of crystals, grains, and individual crystalline cells significantly affects their properties. The creation of heterojunction lasers shows that only quantum mechanics can describe the process on the surface, which is ignored in modern research—for example, [M. Calcagnotto, Y. Adachi, D. Ponge, D. Raabe, Deformation and fracture mechanisms in fine- and ultrafine-grained ferrite/martensite dual-phase steels and the effect of aging, Acta Materialia, (2011) V. 59, Issue 2, 658-670] the interpretation of the obtained results is based on the Orowan stress. The problem of martensitic transition in alloys remains relevant. The main difference between this phase transition and others is its high speed. Using farograms makes it possible to study processes occurring at any speed. A unique role in this transition is played by carbon, whose atoms can form structures from graphite to graphene. The formation of graphene, which has maximum strength at a single-atom thickness, radically changes the strength properties of the material. The advantage of the farogram is that it displays the properties of each chemical element during the destruction of the material.

### Cracking Mechanism

### Warning.

Violation of the integrity of a solid (the formation of pores, cracks, destruction) occurs due to breaking the bond between a finite number of atoms that have absorbed the energy accumulated in a local group of metastable atoms, called the destruction domain. The energy emitted by a separate metastable (excited) atom spontaneously is insufficient to break the integrity. Such an act is possible only with stimulated (induced) radiation, as it happens in a laser. The degree of damage (the number of broken bonds) depends on the absorbed energy. Breaking a bond is an atomic reaction that upsets the dynamic equilibrium so that the atoms move away to a distance of no return.

An atomic reaction due to electron-photon exchange stimulates an atomic-proton reaction, which always occurs according to the abovementioned law.

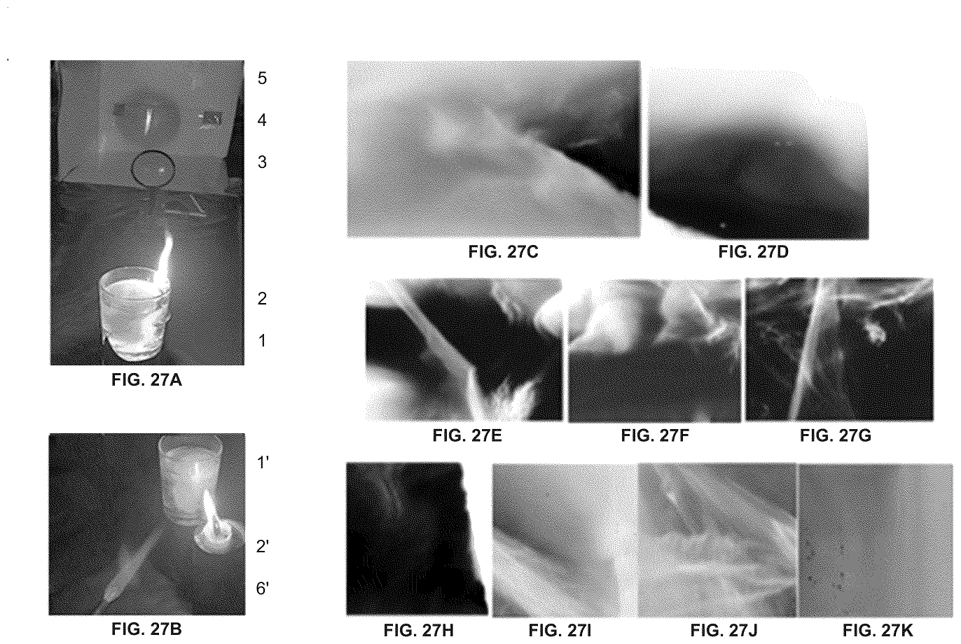

Experience No. 28, FIGS. 27A-K. Quartz glass, cooled with ice to −12° C., was placed before a candle flame. The image of the flame was recorded on a film similar to that shown in FIG. 17. The lower part of the glass was irradiated with the intense flame of a gas burner. The destruction of the glass began from below, located 40 cm below the film. FIGS. 27A-K shows farograms of p-radiation from a candle flame irradiated with p-radiation caused by an explosion of quartz glass. The color of the frame fragments is due to the glow of the photographic material under the action of p-photons.

1. We see nine frames, while visible radiation is only on an opaque container. It is focused, while farograms are observed on both sides of the focus of the visible beams. This fact can only be explained by the fact that the radiation of a candle directed outside the lens is recorded on the film.
2. Each farogram is unique, and no two are alike. Therefore, the radiation is anisotropic in space. This fact can be explained only by external electromagnetic and gravitational influences.
3. These hypotheses are confirmed experimentally.
4. The source of destruction energy (burner flame) was 40 cm below the film and 120 cm further, where red luminous channels converged, similar to lightning. We see them in frames c and e in the direction perpendicular to the channels. This phenomenon is possible only with an electromagnetic pulse. Consequently, a crack in a solid is formed at the speed of an electromagnetic wave.
5. To the right and left of the c-channel, dark bands are observed, indicating the absorption of energy in this region and the formation of a crack. This crack is visible in farograms shown in FIG. 27A and FIG. 27B. The fracture shape is similar to that shown in the Irwin model.

The farograms shown in FIG. 27A and FIG. 27B are particularly interesting, showing at least three color crack tips.

6. The experiment proves that the crack formation mechanism is electromagnetic; the crack ends with a tip but does not begin. An acoustic pulse accompanies this process, the speed of which is 50,000 times less than the speed of light. Just as thunder lags behind lightning. But, unfortunately, the speed of the acoustic pulse in the modern theory of strength is taken as the rate of crack formation.
7. Distinct spectral lines are repeatedly observed. The nature of this phenomenon allows us to conclude that the inventor observed the dispersion of p-radiation. It is known that F. P. Leroux (1860) was the first to observe the anomalous dispersion of light in iodine vapor.

The discovery of p-ray dispersion is of great practical importance. This makes it possible to determine the frequencies of photons and control them by measuring the intensity of the radiation.

Modern fracture mechanics and government standards are mainly based on Irwin's idea. Various equations are proposed relating the rate of crack propagation dl/dN (change in the length of the crack per cycle) with the change in the stress intensity factor. For example,

$$Peris - Erdogan \frac{dl}{dN} = {C ( Δ K )}^{n}$$

or NASGRO. Unfortunately, these equations are erroneous since the dimensions of the right and left sides are different.

Delayed emission from fragments with a delay c indicates that not all metastable atoms of the destruction domain emit stimulated emission. Thus, the photographs illustrate spontaneous emission before and after a fracture. We observe not one crack tip but four, three of which are colored. This means that photons of a specific frequency are absorbed. The blacktip indicates the absorption of photons of all frequencies. Therefore, a crack has formed there. The red area shown in farogram (b) ends with a tip in farogram (a). Blackening is incredibly intense in farogram (c). Consequently, the crack formed there earlier. So, we conclude that cracks end at the tip, the mechanism of crack formation is electromagnetic, and the rate of crack formation is equal to the speed of electromagnetic waves.

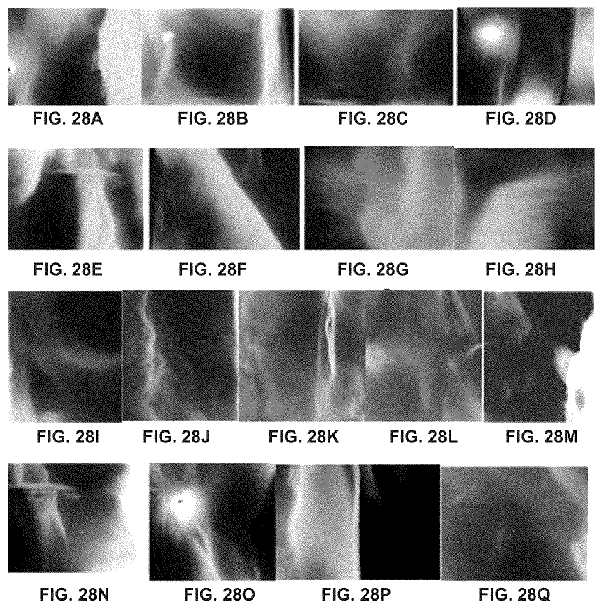

Experiments Nos. 29, 30, 31, 32. FIGS. 28A-Q. Four experiments were performed to obtain farograms for brittle and plastic fracture of a hacksaw blade that was not used before the experiment and subjected to annealing. Two types of deformation were used: bending and impact.

Experiment No. 29, FIGS. 28A-D show brittle fracture in bending.

Experiment No. 30, FIGS. 28E-H show plastic fracture in bending.

Experiment No. 31, FIGS. 28I-M deformations of the unannealed sample.

Experiment No. 32, FIGS. 28N-Q deformation of the annealed sample.

The emission of a p-photon is possible only when an atom is ionized, that is, when an electron is removed from the K or L energy levels close to the nucleus. The inventor postulates that in the absence of an accelerating voltage, the removal of an electron from these levels occurs due to the impact of a proton, the mass of which is 1836 times greater than the mass of an electron. The properties of the proton are unique.

All experiments carried out by the inventor testify to the high sensitivity of p-radiation to external influences, including magnetic ones, when p-rays pass through materials, especially ferromagnetic ones. The beam interference in FIG. 18 was obtained by reflecting light from a mirror in two experiments. Thus, using p-radiation to create instruments like microscopes has no fundamental difficulties. The formation of a boundary separating the dark, non-radiating region from the region in which p-quanta is emitted has been repeatedly observed in the study of processes in inorganic materials and human organs. The appearance of a region that does not emit photons in a living organism requires an explanation, which can only be given with a comprehensive study of organs using modern equipment.

M. Faraday described in the sixth lecture on the history of candles the unique mechanism of carbon combustion, which predetermined the discovery of fullerene, nanotubes, and graphene. The formation of such phases in an inorganic material significantly changes its properties. However, in a living organ, this can lead to the cessation of normal functioning of the molecule.

### Corrosion and Stress Corrosion Cracking

Corrosion is the result of a chemical reaction in which the formed compound destroys the integrity of the product, leading to disaster. In particular, damages leading to depressurization of, for example, the aircraft fuselage, containers with liquid or gas, and pipelines are dangerous.

Forty-two nails, 0.079″ (2 mm) in diameter and 1.54″ (39 mm) long, with an average weight of 0.795 g, were fixed equally on two delicate washcloths 1 and 2, which were attached separately to the inner surfaces of identical hollow steel cylinders. Three small magnets are attracted to the surface of the cylinder wall of one of the cylinders, pressing the bunches against the wall. The induction of the magnets, determined from the lift, was 9:3:1 from left to right.

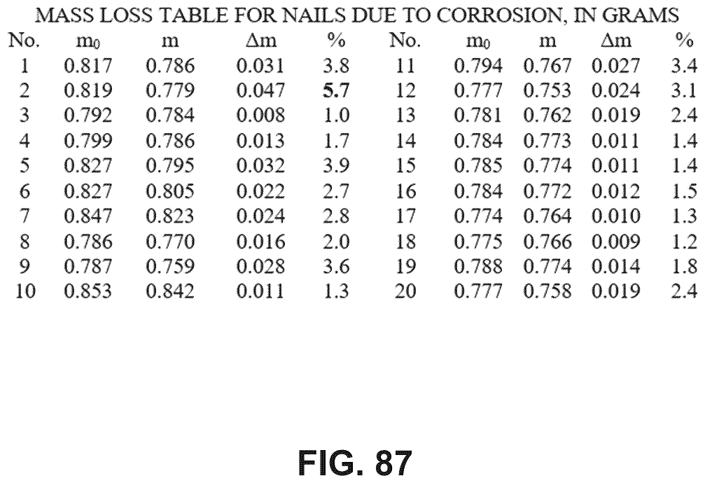

A plastic container with an aqueous solution of NaOCl was located at the bottom of the cylinder at a distance of 15 cm from the nails. The cylinder was closed with a cap, but not hermetically. Thus, metal corrosion occurred under the action of water vapor and chlorine. The experiment lasted 144 hours. The experiment results are shown in the Mass loss table for nails due to corrosion, as shown in FIG. 87. A similar experiment was carried out with nails placed on washcloth 2, but without magnets and film, in another cylinder under the same conditions simultaneously.

Visual analysis makes it possible to smooth out a significant difference in the corrosion rate in and without a magnetic field. There are quantitative estimates of the difference in corrosion rates based on measuring the mass of nails before and after the experiment. The weighing results are shown in the table. The maximum weight loss of nails without magnets was 5.7%. This fact is decisive in most cases, especially when depressurization is already a disaster.

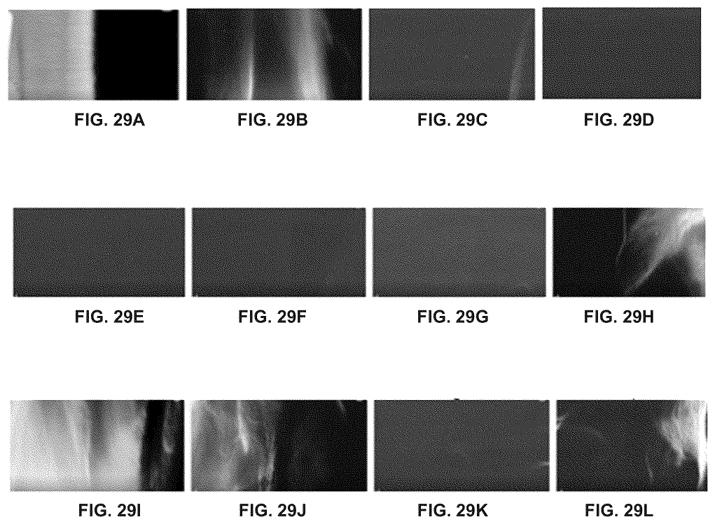

The magnetic field inhibited the chemical reaction that led to corrosion, so much so that the mass of 20 nails fluctuated within the statistical error. The mass of nail No. 21 changed by 0.8%. Visual and weight analysis of nails in a magnetic field showed that the corrosion rate increases with decreasing magnetic induction. The induction of the magnetic field was not sufficient to suppress the nails No. 19-No. 21. Twelve farograms shown in FIGS. 29A-L indicate that p-radiation was used to study the processes occurring in the depth of the material, in which there was no change in the mass and corrosion of the surface. Farograms (a and k) illustrate the surface separating dark and color areas; The farogram shown in FIG. 29H shows the explosion BL. The dark color of farograms, shown in FIGS. 29C-G and FIG. 29K means suppression of chemical processes. However, these conclusions of the inventor require additional verification by other methods.

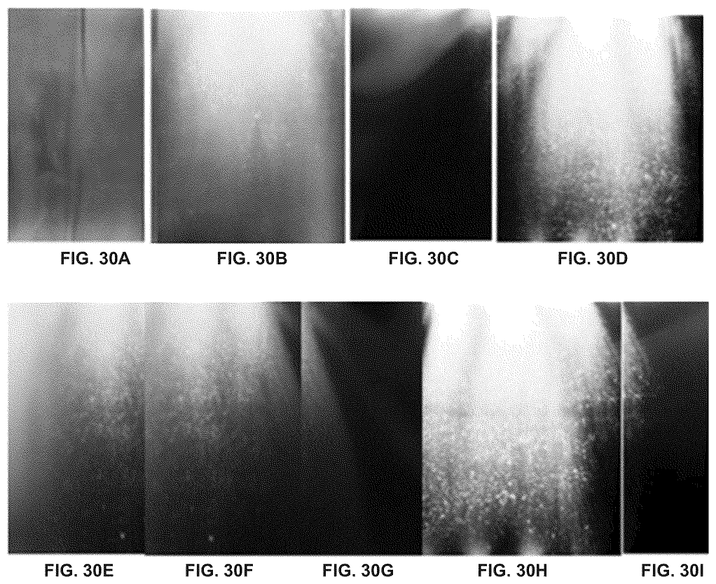

Experiment No. 33, FIGS. 30A-I. The steel cylinder was partially filled with an aqueous solution of NaOCl. The film was located above the surface of the aqueous solution inside the cylinder. The experiment lasted 48 hours. FIGS. 30A-I shows nine farograms taken over the surface of the solution. Corrosion occurred under the action of solution vapor rising from bottom to top. Let us note some features of farograms. Farogram a illustrates a green spectral doublet. The synthesis of water is observed on farograms shown in FIG. 30C, FIG. 30D, and FIG. 30H; farograms shown in FIG. 30d and FIG. 30H have minor differences; on the farogram, an explosion is observed during the synthesis of water.

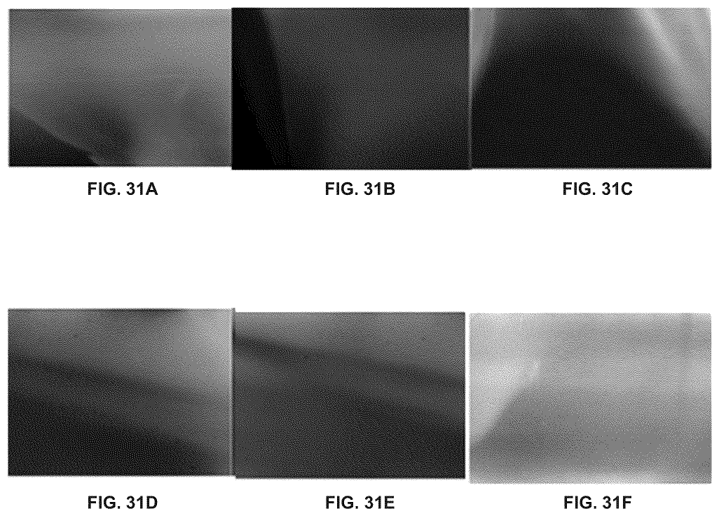

Experiment No. 34, FIGS. 31A-F. The same steel cylinder was partially filled with an aqueous solution of NaOCl. The film was located on the outer surface of the cylinder so that it was located above the surface of the aqueous solution. A permanent magnet 78 mm in diameter was placed on the film surface. The experiment lasted 48 hours. The farograms presented in FIG. 31 show the changes that have occurred due to the influence of a magnetic field.

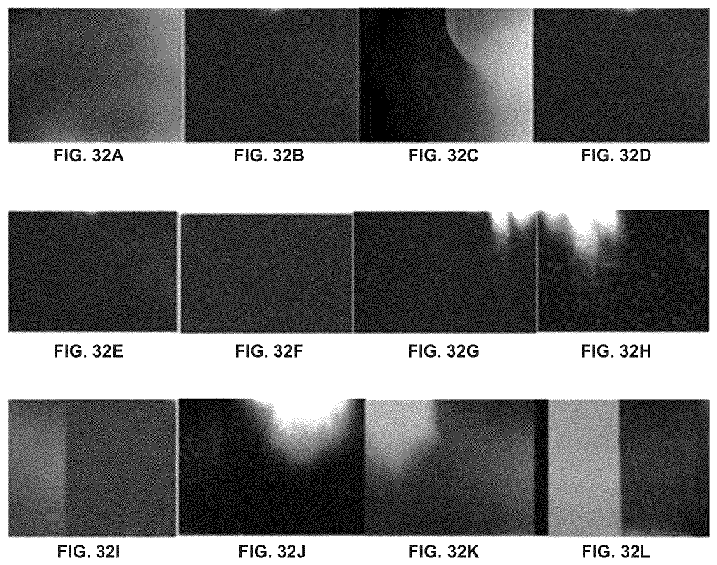

Experiment No. 35, FIGS. 32A-L. This experiment differed from the previous one in that metal corrosion was studied in a solution, not above it.

Twelve farograms demonstrate the effect of a magnetic field on chemical reactions occurring in a product that simulates a pipeline. The magnetic field inhibited the radiation, as shown in most farograms, but did not completely suppress the reaction. For example, the synthesis of water, shown in farograms (g and h), is accompanied by red emission, which indicates the absorption of higher photon frequencies. A similar pattern is observed in farogram (j). However, in the dark region (j), water synthesis occurred. The inventor demonstrates the possibilities of the method based on experimental studies carried out without modern equipment.

However, the proposed method does not exclude the possibility of studying processes simultaneously with other methods since it does not require a vacuum and has a high penetrating power.

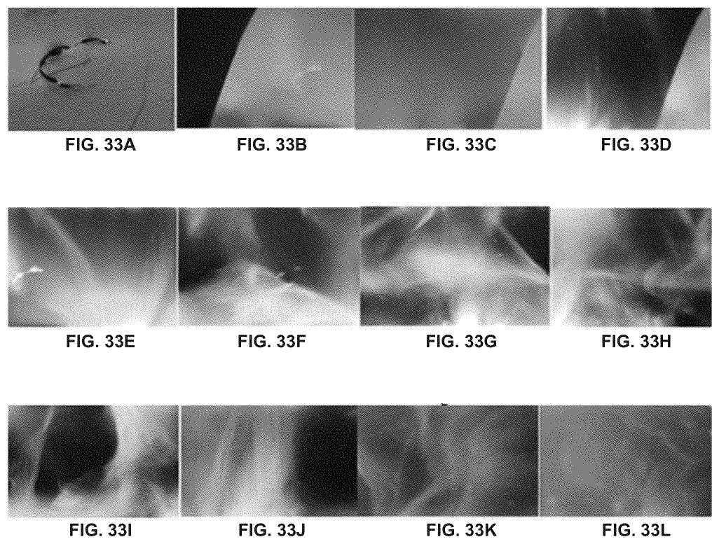

Experiment No. 36, FIGS. 33A-L. A steel cylinder 9 inches (228.6 mm) in diameter, 2 feet (609.6 mm) long, and 0.118 inches (3 mm) thick in the wall, having a bottom, longitudinal, and transverse seams at a 45° angle to the ground open hole. The sandblasting nozzle was located inside the cylinder 5 inches (127 mm) from the bottom. Sand at a pressure of ˜1500 psi (10.34 MPa) was sent to the center of the face.

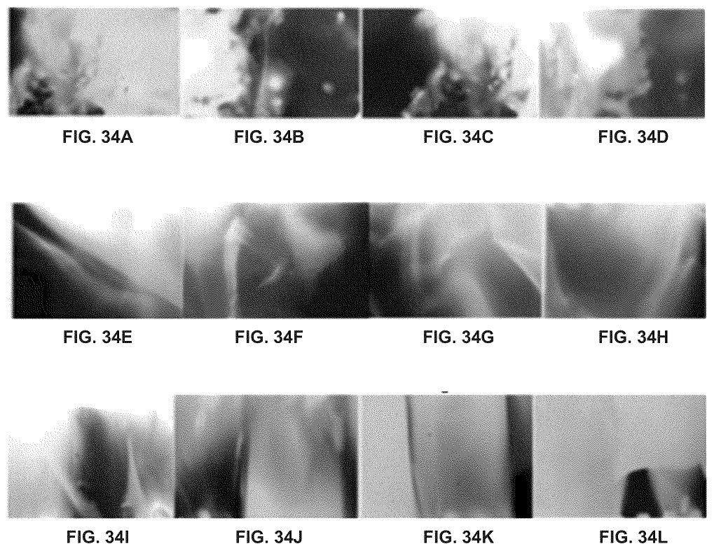

Two films were U-shaped on the outer surface of the cylinder and the lid in mutually perpendicular planes: top-bottom (FIGS. 33A-L), 2) north-south (FIGS. 34A-L), so that their centers coincided with the center of the lid. Thus, each of the three parts consisted of four frames. The experiment lasted 4 minutes. The test result is shown in FIGS. 33A-L and FIGS. 34A-L. The farograms shown in FIGS. 33E-H and FIGS. 34E-H of both films fix p-radiation from the lid caused by the ingress of sand on the inner surface of the lid. The remaining frames register p-radiation from the side surfaces of the cylinder. I draw your attention to three essential facts. First, farograms taken in two mutually perpendicular directions differ. Such a difference was observed in FIGS. 7A-F, FIGS. 8A-F, FIGS. 9A-F, and FIGS. 10A-F. Secondly, the film is intentionally damaged in the place where the farogram, shown as FIG. 33A, is located. The damaged image is reproduced on farograms shown in FIG. 33B, FIG. 33E, FIG. 33F, FIG. 34B, FIG. 34E, and FIG. 34F. Thirdly, the form of farograms shown in FIGS. 33B-D and FIGS. 34B-D are identical. These repetitions cannot be random, and we must explain them.

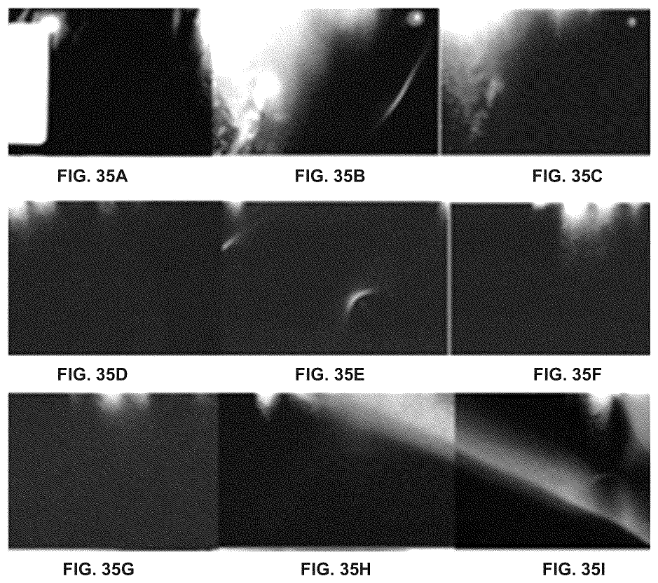

Experiment No. 37, FIGS. 35A-I. A jet of water at a pressure of 1500 psi (10.34 MPa) was directed into the pipe on the lid for 200 seconds. The container with the film was placed on the surface of the cylinder in a U-shape in such a way four frames were placed on the lid and side surfaces. The test result is shown in FIGS. 35A-I. The farograms obtained on the outer sides of the cylinder are due to the impact of water reflected from the lid. Farograms shown in FIGS. 35A-C illustrate intense radiation during BL generation and water synthesis, farograms shown in FIGS. 35D-I illustrate water synthesis against the background of BL. There is an intense glow on the farograms shown in FIG. 35H and FIG. 35I, which was not previously recorded.

FIGS. 35A-I. The farograms obtained on the outer sides of the cylinder are due to the impact of water reflected from the lid. The farograms shown in FIGS. 35A-C illustrate intense radiation during BL generation and water synthesis. The farograms shown in FIGS. 35D-I illustrate water synthesis against the background of BL. There is an intense glow on the farograms shown in FIG. 35H and FIG. 35I, which was not previously recorded.

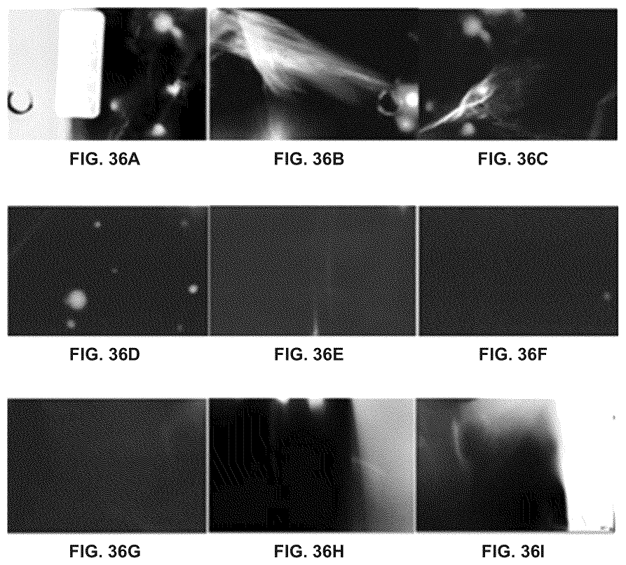

Experiment No. 38, FIGS. 36A-I. The sandblasting nozzle was located inside the cylinder 5 inches (127 mm) from the bottom. Sand with water at a pressure of ˜1500 psi (10.34 MPa) was sent to the center of the lid. The experiment lasted 200 seconds. The result of the experiment is shown in FIGS. 36A-I.

FIGS. 36A-I. The farograms obtained with the simultaneous action of sand and water on the metal show that energy redistribution has occurred. The yellow and red color of defects indicates that high-frequency photons are absorbed. Point defects indicate a high concentration of energy, at which the risk of pitting corrosion and pipeline depressurization increases. Let's pay attention to the fact that the film is damaged in the farogram shown in FIG. 36A, and its display is recorded in the farogram shown in FIG. 36B, on which there is no damage. Thus, we observe an effect that does not exist. Such a phenomenon is well-known in optics. It is called a mirage. But on this farogram and the farograms shown in FIGS. 33A-L, damage does not disappear, as in a quantum mirage.

The study of farograms allows you to establish the area of additional applications.

The color of each object shown on the farograms is due to the frequencies of p-photons emitted when an electron fills the energy level K or L of a chemical element or atoms of various elements. The description of the mechanism of K or the formation of farogram elements is possible only with the help of quantum mechanics. Nanotechnology became possible after a series of experiments. One such experiment was the discovery of a quantum mirage in which a cobalt atom placed at the focus of an ellipse formed by 36 other cobalt atoms produced an actual signal at a second focus where the atom was not present. This effect became the basis for the creation of quantum computers. P-photons propagate not only in conductors but also in a dielectric, which makes it possible to create devices at the atomic level without conductors. We will assume that the channels between the yellow BL can be both straight and with such a bend.

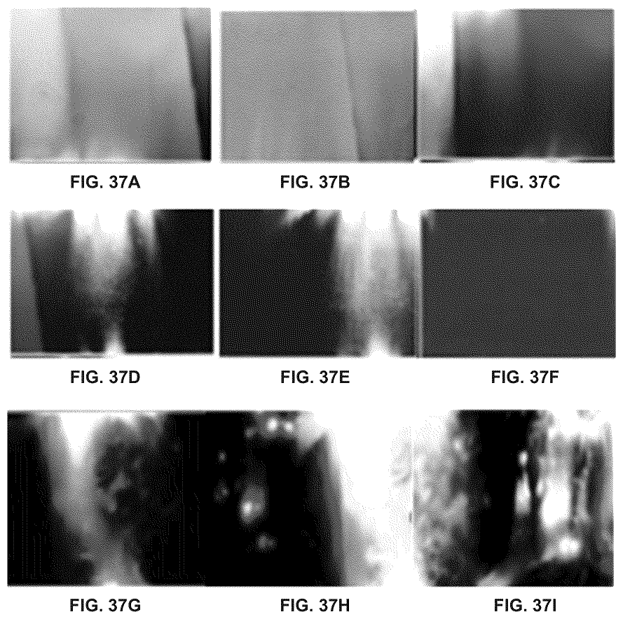

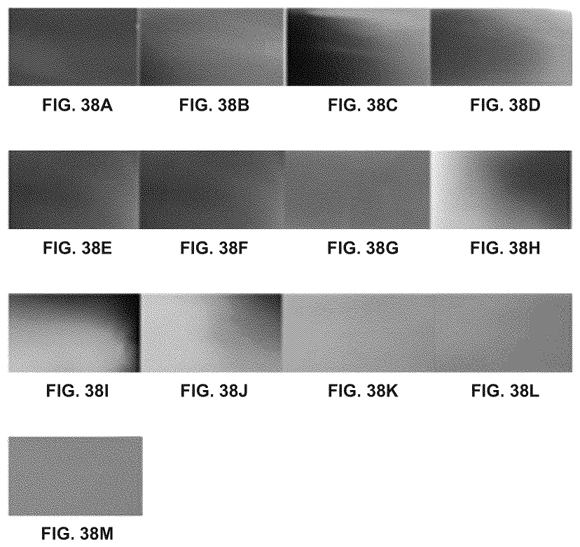

Experiment No. 39, FIGS. 37A-I. A 2″×3″×¼″ steel angle was used to study impact farograms. The Film was attached to the horizontal bottom surface (XY). The blow of the ax is applied on the upper surface (XY) in the Z direction. The farograms shown in FIGS. 37A-E illustrate the synthesis of water, which is intense in the farograms shown in FIG. 37D and FIG. 37E. Water synthesis occurred in the dark area, separated by a flat boundary from the red one. In addition, it is necessary to pay special attention to the shape of the white radiation on the farograms shown in FIG. 37D and FIG. 37E. Experiment No. 40, FIGS. 38A-M. The thirteen farograms shown in FIGS. 38A-M were obtained in an experiment that differed from the previous one in that a permanent magnet 4×30 cm² in size was placed on the Film. The features of the farograms presented in FIGS. 37A-I and FIGS. 38A-M make it possible to explain the mechanism of the influence of an impact on the change in their color under the action of a magnetic field. The impact results in a displacement of the ions, in which the displacement of the electron cloud is delayed. The white color of BL indicates the absence of absorption of emitted p-photons in the region, and the red color indicates the absorption of photons with a higher frequency. According to Stokes' law, the frequency of photons in the visible region is less than that of p-photons absorbed by the photographic emulsion. The magnetic field changes the trajectory of the electrons. It prevents the formation of local areas of energy accumulation, as shown in FIG. 38. However, p-photons, which cause luminescence in the visible region, make up only an insignificant part of the total emission of p-photons. The maximum number of n-photons absorbed by the metal causes luminescence in the high-frequency region of the spectrum.

### Note.

The magnetic field vector is perpendicular to the ax's velocity vector.

This experiment is of particular importance. Almost uniform energy distribution over the entire surface eliminates the possibility of stimulated radiation, cracking, and destruction. The absence of regions with a high concentration of radiation energy, characteristic of water synthesis, can only be explained by the fact that in a magnetic field, the radiation of excited atoms is incoherent, and the destruction domain (local region of metastable atoms) is not formed.

The Genesis of Human Organs and their Monitoring

An analysis of numerous experiments performed by the inventor allows us to conclude about the unique properties of protons that appear when interacting with protons, neutrons, electrons, and atoms. Linear spectra accompany the formation of a neutral atom of a proton with an electron due to the monochromatic radiation of electromagnetic waves. Regularities in the spectrum of hydrogen were established by N. Bohr in 1913, who formulated the first quantum postulates that refute the ideas of classical electrodynamics. Quantum mechanics has been created, with the help of which the spectra of many-electron atoms are described-based on quantum postulates. Farograms obtained using p-radiation demonstrate individual spectral lines and multi-colored regions for all processes in inorganic and organic materials. P-photons, which cause visible radiation, comprise only a part of the spectrum. Several metals have been used as screens, which are irradiated with particles formed by the destruction of metal or glass in contact with a rotating whetstone. The time of delayed radiation by particles that have broken off from the material was 10⁻³-10⁻⁴ s.

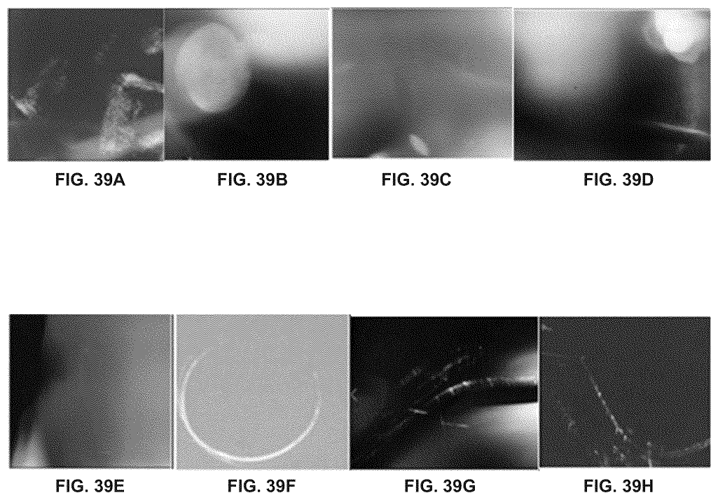

Experiments No. 41-47, FIGS. 39A-H. The farograms are shown in FIGS. 39A-H obtained under the following conditions: Aluminum foil in one, four, and eight layers, located on the surface of the container, was irradiated with p-rays emitted from iron alloy particles; b) a steel washer located on the surface of the container was irradiated with p-beams emitted from copper alloy particles; c) the same puck was irradiated at double exposure and e-aluminum foil, located on the surface of the container (shown at the bottom left), was irradiated with p-beams emitted from copper particles formed after destruction; f-Gartl's steel washer was irradiated with radiation from steel particles.

The samples above the film surface shielded it from direct radiation. The use of alloy screens does not indicate any fundamental obstacles to creating sensors for studying the p-radiation spectrum. The absorption of photons causes the glow of metals, including p-luminescence, the spectrum of which depends both on the frequency of the radiation source and on the absorber. The farograms show that the number of combinations of radiation source-absorber is not limited.

This makes it possible to assemble new sensors for studying p-radiation and use ready-made equipment and well-established programs in technology or medicine.

A broad spectrum of radiation and luminescence, to some extent associated with destruction, predetermines the possibility of predicting changes in the wear of a solid during its operation since the destruction mechanism has been disclosed. Detecting farograms allows you to create new, more accurate control methods. The high penetrating power of p-radiation and intense luminescence is because the proton's mass is 1836 times greater than the mass of the electron. This is observed for all types of solid body deformation. The inventor believes that such sources for plants and organs are the electromagnetic and gravitational influence of the Sun and the gravitational influence of the Moon. This hypothesis was formulated based on farograms shown in FIGS. 7A-F, FIGS. 8A-F, FIGS. 9A-F, and FIGS. 10A-F.

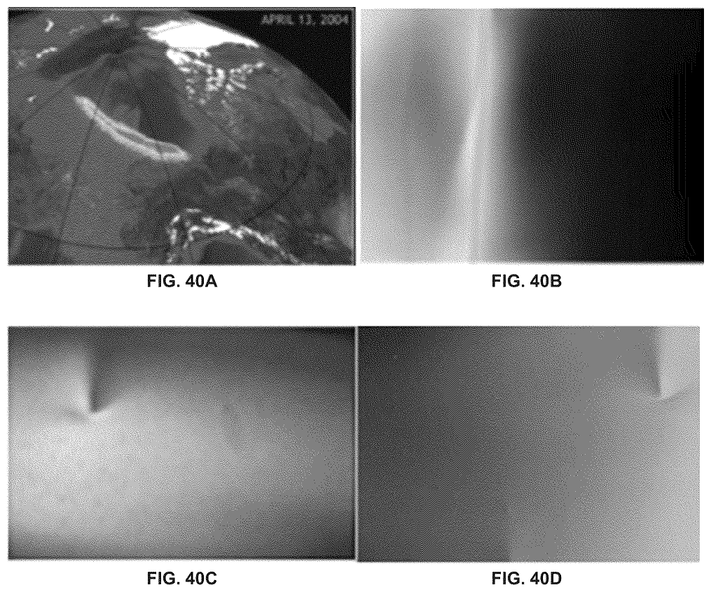

Experiment No. 42, FIGS. 40A-D. FIGS. 40A-D illustrate the results of four studies: FIG. 40A—“X-ray image of the north polar regions of Earth obtained by Chandra HRC-I showing large variability in soft (0.1-10.0 keV) X-ray emissions from Earth's aurora. The bright arc in this Chandra image shows low-energy X-rays generated during auroral activity.” [See: A. Bhardwaj, M. Lisse, K. Dennerl, X-Rays in the Solar System, Encyclopedia of the Solar System, (2014), 1019-1045]; FIG. 40B—the Film was placed on a concrete surface under a steel plate 6 mm thick, exposed to external influences, including sunlight, for 144 hours. We see in the photograph two spectral lines (bright blue and faint, probably red), the source of which is unknown; FIG. 40C and FIG. 40D—the two figures shown on the mirror frames are symmetrical. The image shown in farogram, FIG. 40C, is obtained by hitting the metal, while (d) is obtained by repeating the previous experiment. Let us pay attention to the fact that there is a border between multi-colored areas in all cases. This allows us to conclude that the photograph of the spectral lines and the result of the inventor's experiments are due to p-radiation since X-rays at such energy will not pass through the screen.

Using p-radiation in medicine and biology makes it possible to study processes at the atomic level without harming a plant or organ. The analysis shows that identical farograms display some processes in organs and inorganic materials. The reaction occurred in the crystal's unit cell or part of the biomolecule.

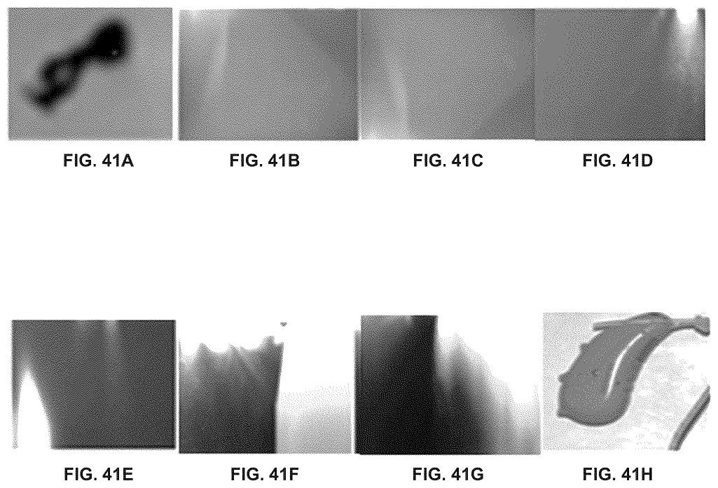

Experiment No. 43, FIGS. 41A-H. Several farograms of p-radiation from the organs of the inventor were demonstrated in his patent. They are unique. However, the results of new experimental studies of the p-radiation mechanism, especially by living organs, make it possible to use this radiation for more accurate, non-invasive monitoring of organ pathologies caused by external influences, including viruses.

The Film was placed on the head of the inventor in a circle at the level of the forehead. The experiment was performed during a night's sleep for two hours. Fourteen farograms were obtained, seven shown in FIGS. 41A-H, demonstrating two properties of p-radiation. The farograms shown in FIGS. 41B-G illustrate the synthesis of water, while in the farogram shown in FIG. 41A, we see blackening due to the absorption of p-radiation by the metal part of the hearing aid caught between the head and the Film. P-radiation passed through the plastic body of the HA, as shown in FIG. 41H.

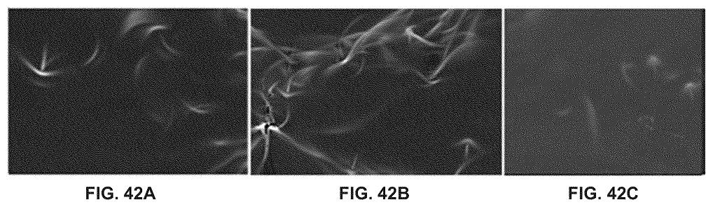

Experiment No. 44, FIGS. 42A-C. Three of the fourteen farograms obtained during the experiment illustrate photographs of the inventor's heart and vessels after three cardiac catheterization tests and bypass surgery.

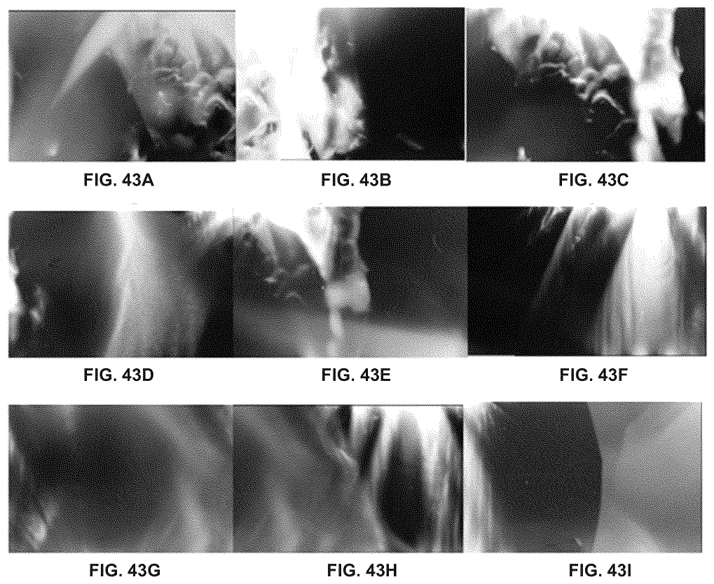

Experiment No. 45, FIGS. 43A-I. The Film was placed on the inventor's chest at the right atrium, left heart, ear, and pulmonary artery level. The location of farograms can be determined because the frame (h) is above the sternum. The experiment lasted two hours in the evening. Farograms (ab and c) are identical but formed at different frequencies of p-photons. Farograms were obtained by the inventor the day before the fourth cardiac catheterization test to understand the process they characterize, as was the case with water synthesis. X-rays, with which the cardiac catheterization test is performed, kill the living cell, while p-rays are non-invasive. But there are no obstacles to the simultaneous study of vessels using p-rays and X-rays to find a way to refuse X-rays.

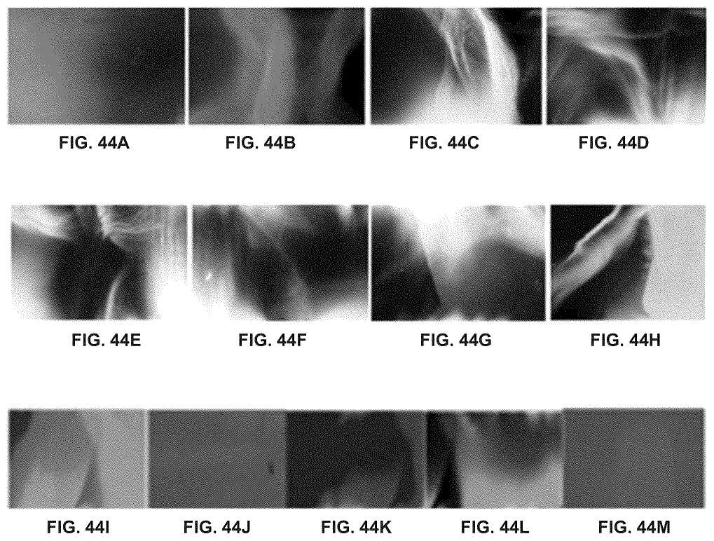

Experiment No. 46, FIGS. 44A-H. Eight farograms, shown in FIGS. 44A-H, illustrate the result of the earache test of the inventor's granddaughter. The ear is depicted in the farograms shown in FIG. 44B and FIG. 44C. The colored vertical and horizontal lines are depicted in the farograms shown in FIGS. 44C-F. Water synthesis is observed on the farograms shown in FIG. 44F and FIG. 44G; there is a need to pay attention to the intense radiation from the spinal cord, depicted in the farogram shown in FIG. 44E. The experiment was performed during sleep for one hour.

Experiment No. 47, FIGS. 44I-M. Five farograms, shown in FIGS. 44I-M, illustrate the result of the study of p-radiation from the eyes of the inventor's son. The distance between the dark pointed peaks on farograms, shown in FIG. 44I and FIG. 44L, equals the interpupillary distance. Water synthesis is observed on the farograms shown in FIG. 44k and FIG. 44L. The third image of the pupil, depicted in the farogram shown in FIG. 44k, is difficult to explain as a shift due to the different background colors. The experiment was performed during sleep for an hour.

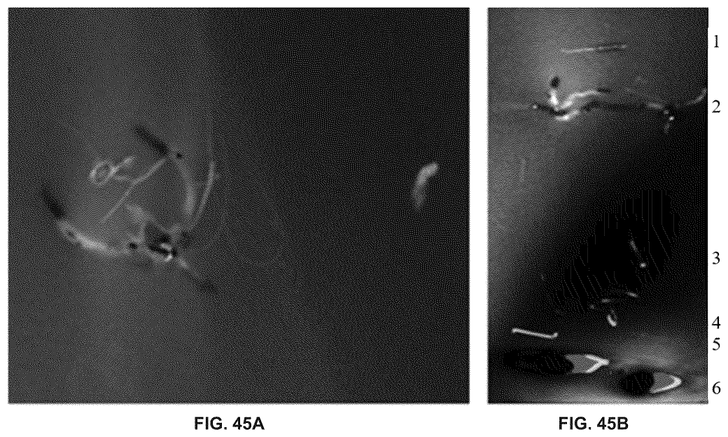

Experiment No. 48, FIG. 45A. The farogram illustrates a parasite found in the inventor's stomach during a seven-hour sleep. The Film was located on the surface of the body.

Experiment No. 49 FIG. 45B. The experiment was repeated during the working day with a two-hour exposure.

Fragments of the farogram make it possible to trace the process of the emergence of the parasite and its development in the stomach of the inventor in individuals at different stages of genesis.

Analysis of the photographs presented in FIG. 45A and FIG. 45B shows that all processes of the origin of cells of a living organism and its development, including the human brain, are accompanied by p-radiation, which can be recorded using farograms.

A fragment shown in FIG. 45A and five fragments shown in FIG. 45B allow helminthologists and parasitologists to control the emergence, development, and harmful effects of parasites to destroy them. Analysis of farograms allows us to conclude the mechanism of physical processes, the growth rate, their number, and the effect of the destruction of parasites. The blue color, indicating the high energy of the process of the birth of a living organism, is observed in the insect organ shown in FIG. 45B, stage 2. Weak plasma radiation is observed above it. Further development can be seen in FIG. 45B. Stages 2→4→3→5→1 indicate the birth of an individual and its development. Stage 6 probably illustrates bacteria. Modern instruments make it possible to detect electromagnetic radiation tens of thousands of times weaker. Using these devices and high-speed photography, the researcher can obtain information about the origin and development of the organ, sufficient for more accurate conclusions.

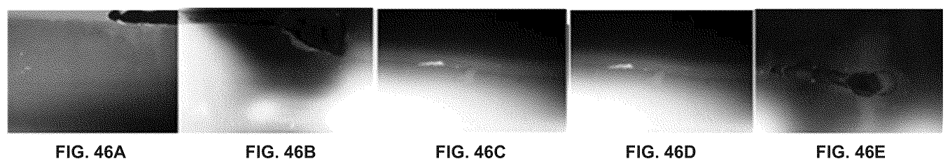

Experiment No. 50, FIGS. 46A-E. The five farograms shown in FIGS. 46A-E illustrates the genesis of a fly larva (Calliphoridae) in a rotting fish born outside the Film (farogram e). As we can see, the size of the larva has increased, and it has moved ˜10 mm. The work [See: S. V. Nesterov, N. S. Ilyinsky, V. N. Uversky, Biochimica et Biophysica Acta (BBA)—Molecular Cell Research, V. 1868, Issue 11, October 2021, 119102] is devoted to liquid-liquid phase separation in living cells.

S. V. Nesterov, corresponding author of the study, said: “It has been shown that liquid-liquid phase separation processes occur in the cells of living organisms, which control a wide variety of biological processes. The controlled process of forming a new phase and interphase boundaries can be used to transmit signals within a cell or between cells.” The mechanism of the detrimental effect of parasite bacteria on zombies' plants is described in work [See: Huang et al., Parasitic modulation of host development by ubiquitin-independent protein degradation, Cell (2021), https://doi.org/10.1016/j.cell.2021.08.029], which is designed to protect plants.

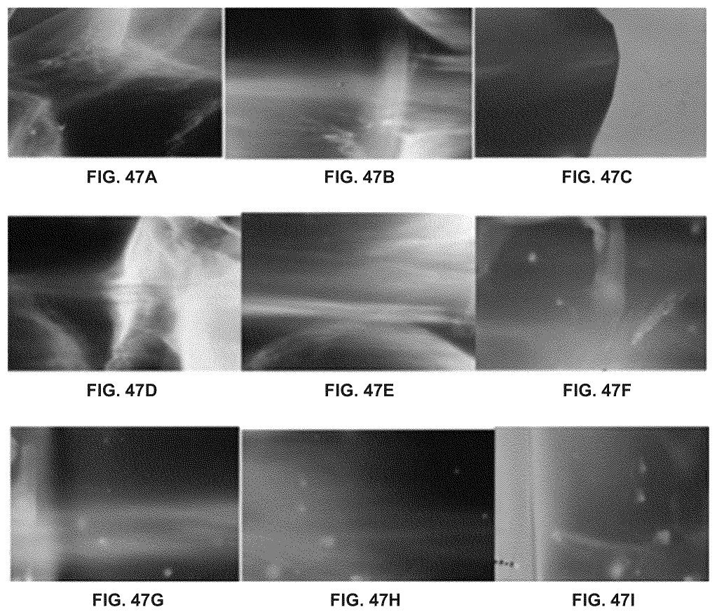

Experiment No. 51, FIGS. 47A-I. FIGS. 47A-I illustrates the emission of a candle flame under the influence of a constant magnetic field and the p-radiation of the inventor's hands, located between the poles of the magnet and candles, imitating concave mirrors irradiating the candle flame. The experimental setup is similar to that shown in FIG. 17, where a person replaced the router. However, the farograms obtained in this experiment are different from all the others. Here, attention should be paid to the predominance of brown, the absence of a fan pattern typical of water synthesis, the abundance of BF, the absence of white radiation, and the vertical spectral line on the frame shown in FIG. 471.

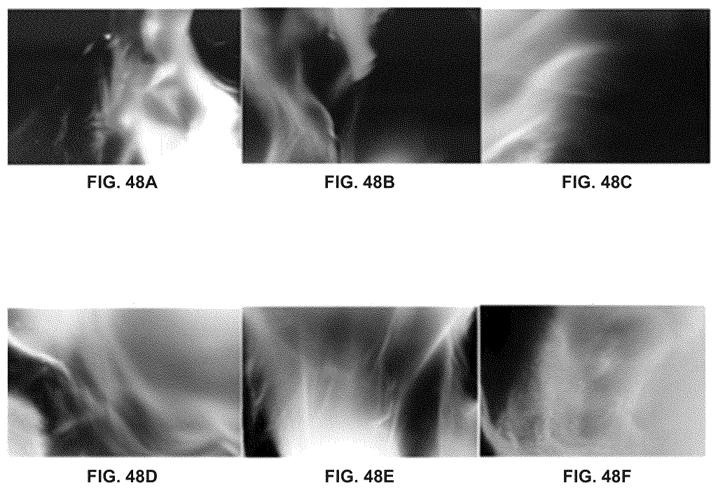

Experiment No. 52, FIGS. 48A-F. FIGS. 48A-F illustrate six farograms obtained in a similar experiment by the inventor's son but without magnets.

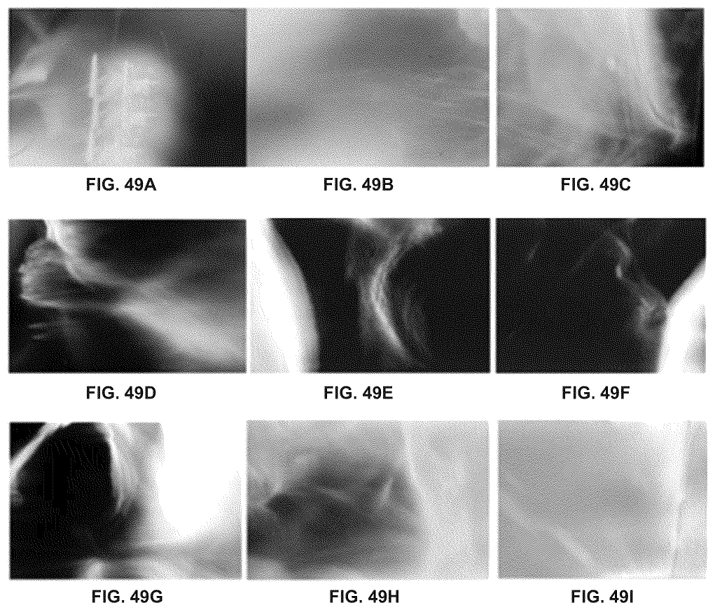

Experiment No. 53, FIGS. 49A-I. FIGS. 49A-I illustrate nine farograms obtained in a similar experiment the inventor's wife performed, but the candle flame was irradiated with p-radiation from the forehead. Similar experiments were conducted with family members who wished to receive their farograms. Each experiment lasted four minutes.

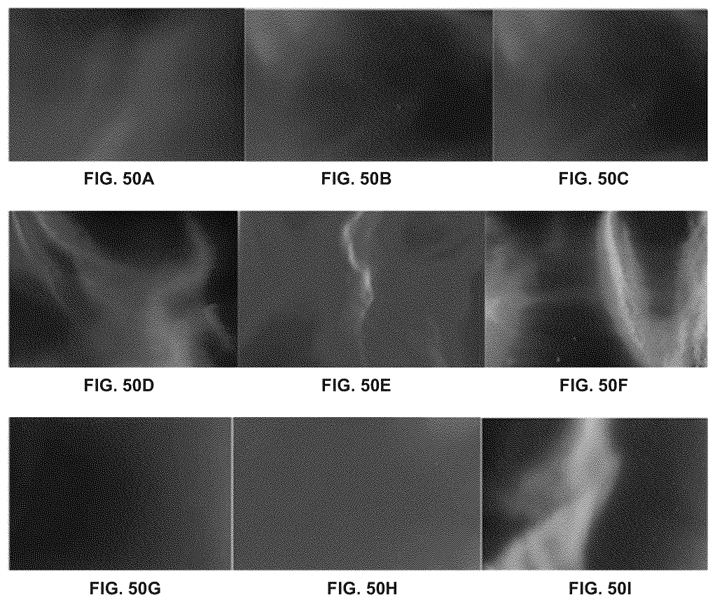

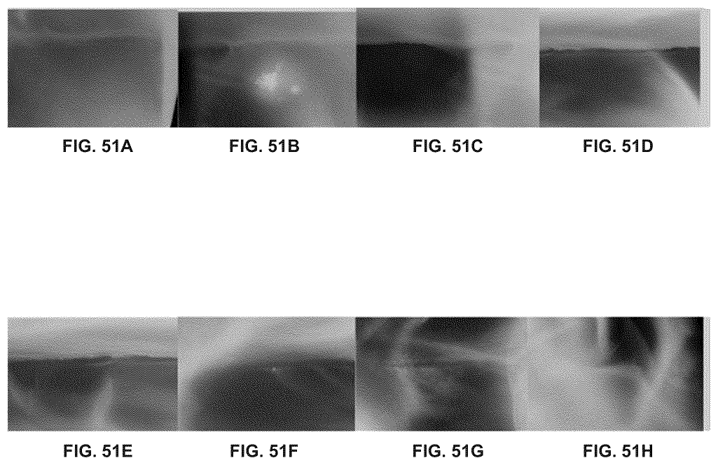

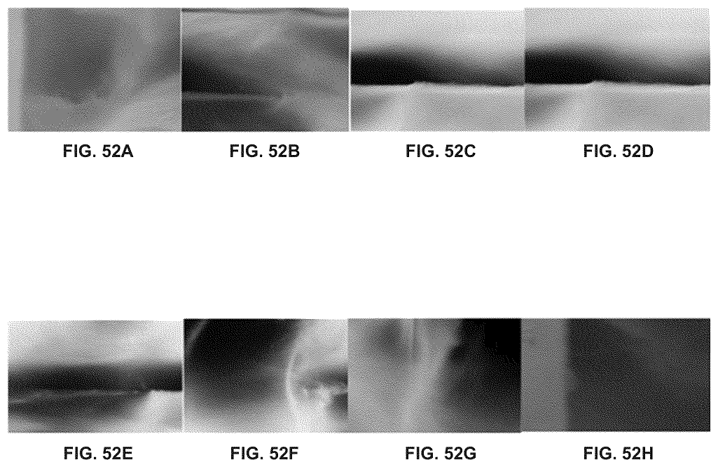

Experiment No. 54, FIGS. 50A-I. Manufacturers offer the INFRAMAT PRO device for infrared heating mats filled with natural gemstones. Unfortunately, the device's inventors do not know that it is not so much the infrared radiation of precious stones that warms the body and has a beneficial effect on the human body but the radiation that penetrates it. FIGS. 50A-I show nine farograms of p-radiation characterizing the frequency (energy) of p-radiation. Infrared rays do not cause a photochemical reaction, while the violet (a-d and g) and blue (e and h) colors of the farogram indicate the high energy of the p-photons that excited it. The P-rays emitted by this device the inventor used to eliminate the problem associated with a sharp exacerbation of inflammation of the ligaments on the right leg from the spine to the heel, which caused severe pain, depriving him of regular sleep and the ability to move. Traditional medicines and therapy did not eliminate the pain within a month. Eight farograms presented in FIGS. 51A-H illustrate the state of the leg from the knee joint to the hip. Complete elimination of the problem and the ability to walk came after 32 nights of sleep with the device turned on. Psoriasis was eliminated after 47 sessions; pain in the left hip joint—52; spinal pain—by 56. At the same time, problems with the intestines, urology, and uninterrupted sleep were eliminated.

The successes of modern medicine are mainly due to the creation of physical methods for controlling the processes occurring in the body based on scientific discoveries. Damage to the brain during its study, for example, cartography using electrodes or X-rays, is hazardous. Using p-radiation makes it possible to abandon methods and devices in which an organ, or even a single cell, is damaged.

The fundamental difference between a farogram and a radiograph is that p-photons do not damage the living cells of the organs that study them. As you know, the DNA helix was deciphered using X-ray diffraction. Consequently, the organic molecule has become inorganic. But living DNA emits a set of frequencies that is unique to each individual. Thus, the farogram of the DNA test is individual. The inventor conducted a DNA test on the relationship of six family members for four generations to find a similar farogram. Of particular interest is the inventor's wife's test.

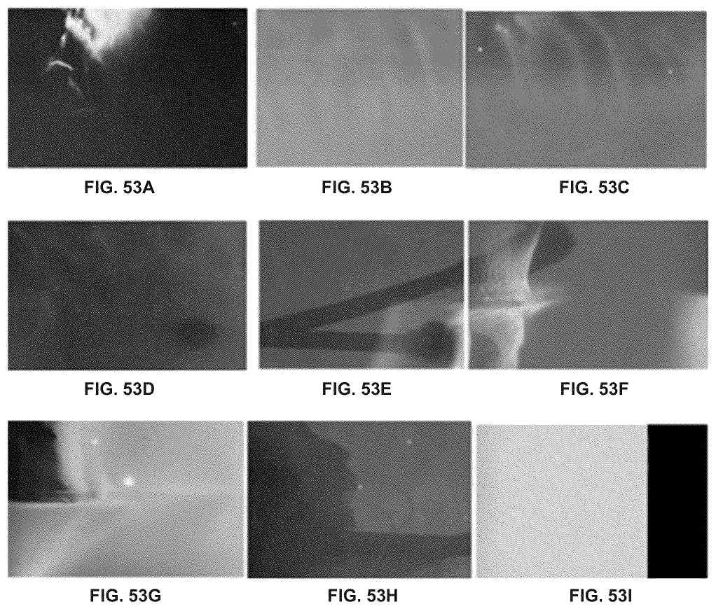

Experiment No. 57, FIGS. 53A-I. The test was carried out according to the generally accepted method: the composition of the liquid wetting the tampon inside the cheek was studied. After wetting, the swab was placed on the film's surface in the dark and left for one hour. The test was performed using two swabs. The simultaneous use of two tampons is accompanied by an interaction that leads to the formation of a luminous region, the processes of which are difficult to explain. We see interference with two minima and three maxima, but this is possible if the two sources are coherent, even if they are different. Therefore, it should be assumed that the radiating atoms retain a constant phase separation.

We observe p-radiation from the investigated liquid, just as from a living organ, for example, as shown with FIG. 41E and FIG. 41F; FIG. 43A, FIGS. 43C-f, and FIG. 43h; FIG. 44E;

FIG. 45B, stage 2; FIG. 47D; and FIG. 48A.

The farogram shown as FIG. 531 of these tests is chosen for comparison, as it is repeated in all six tests. Each farogram is divided by a transparent border, and the color on both sides differs. Each two-color farogram is unique in that the color of one part is different for each person.

The coincidence of shape and color (photon emission frequency) of farograms for inorganic and organic objects is possible only when the photon is emitted not from valence levels but deeper ones. This fact once again confirms the inventor's hypothesis about the p-radiation mechanism.

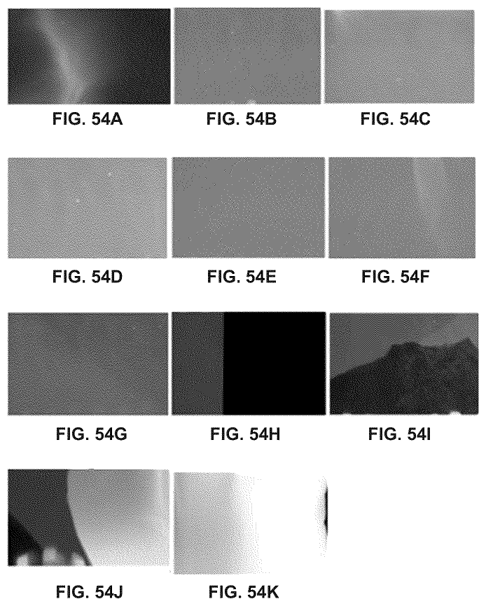

Experiment No. 58, FIGS. 54A-K. Eight farograms illustrate the test obtained by the inventor similarly. The farogram shown in FIG. 54H is used for comparison.

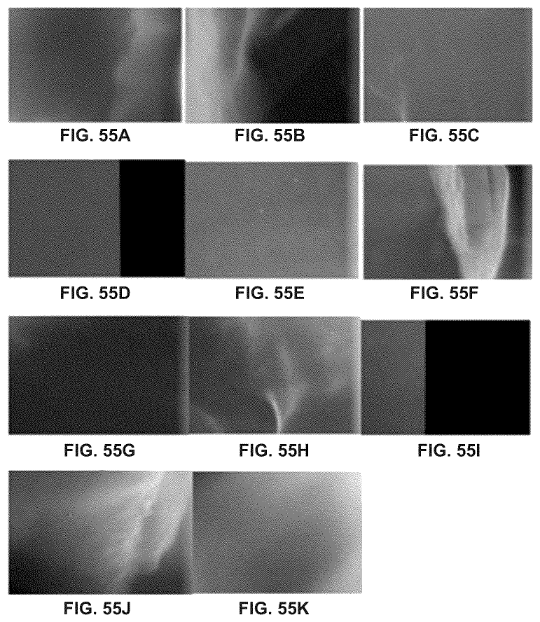

Experiment No. 59, FIGS. 55A-K. Eight farograms illustrate a test similarly obtained by the inventor from his son. The farograms shown in FIG. 55D and FIG. 551 are used for comparison. Three farograms illustrate a test similarly obtained by the inventor from his son's third daughter. The farogram shown in FIG. 55C is used for comparison.

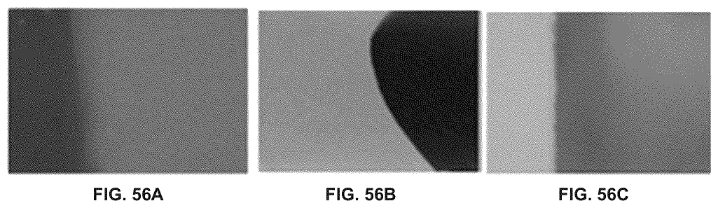

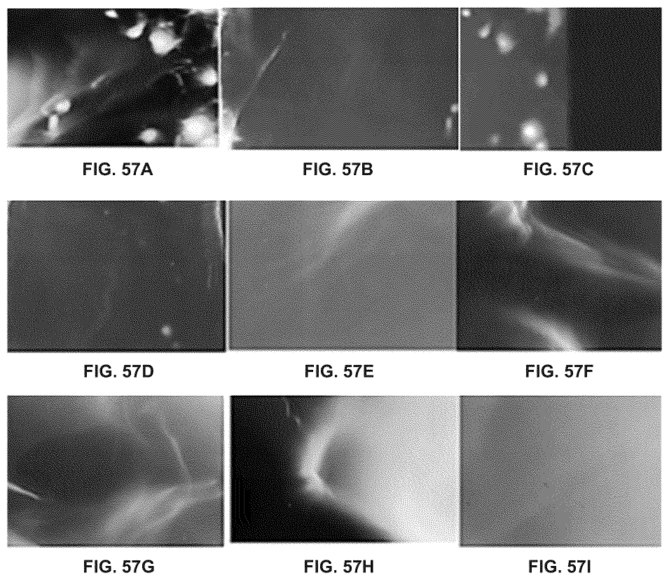

Experiment No. 61, FIGS. 56A-C and FIGS. 57A-I. Nine farograms illustrate a test similarly obtained by the inventor from his son's first daughter. The farogram shown in FIG. 57C is used for comparison.

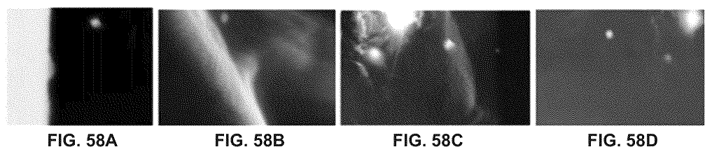

Experiment No. 62, FIGS. 58A-D. Four farograms illustrate a test similarly obtained by the inventor from his son's grandson. The farogram shown in FIG. 58A is used for comparison.

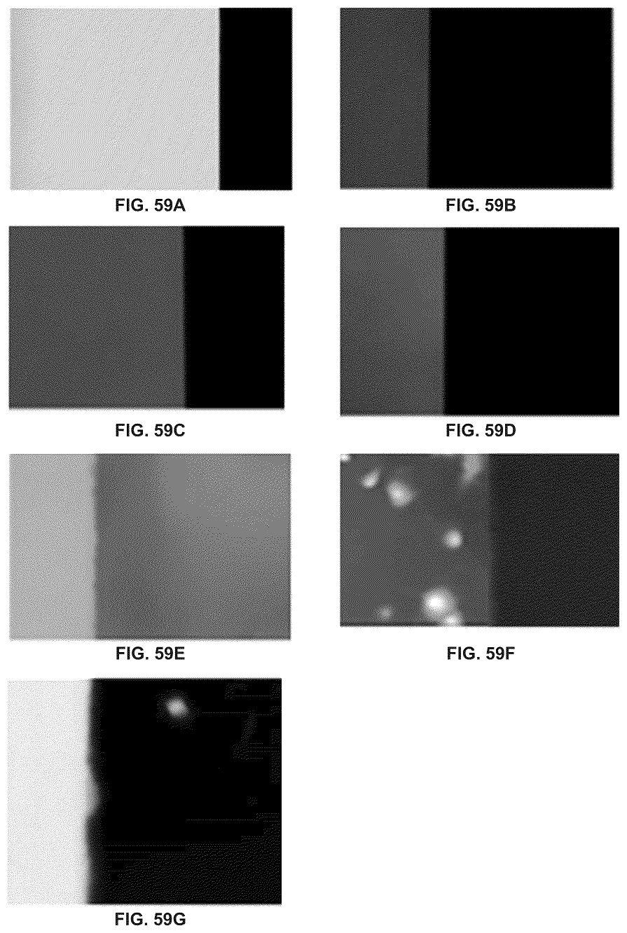

FIGS. 59A-G show a summary table of relationships established based on DNA. FIG. 59A shows a farogram based on a mother. FIG. 59B shows a farogram based on a father. FIG. 59C shows a farogram based on the mother's son. FIG. 59D shows a farogram based on the father's son. FIG. 59E shows a farogram based on the father's third daughter. FIG. 59F shows a farogram based on the father's first daughter. FIG. 59G shows a farogram based on the mother's son.

The experiment is intended to demonstrate the possibility of using p-radiation to analyze processes in a living organism. The relationship test should be considered as an example. The number of possible farograms, limited by the size of the film, is unlimited. But the interpretation of each farogram allows it to be used to develop new methods.

Distinctive Features of Viruses and the Ability to Fight them

The inventor studied the possibility of detecting the virus using p-radiation for a conventional test for people not infected with it. As in previous experiments, the swab dipped in the nose was placed on photographic film in the dark and exposed for an hour.

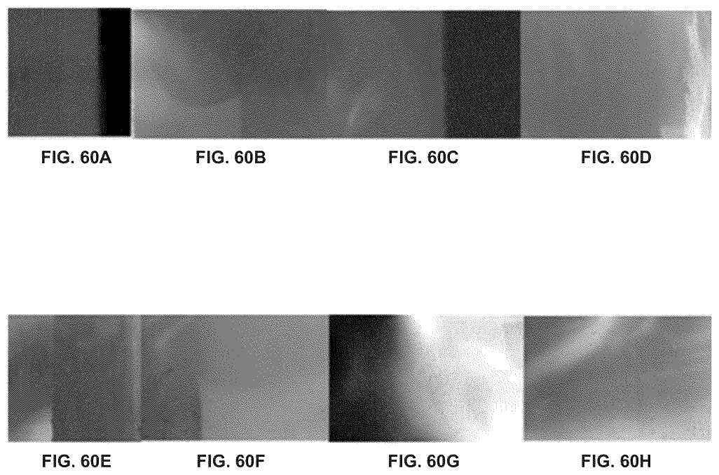

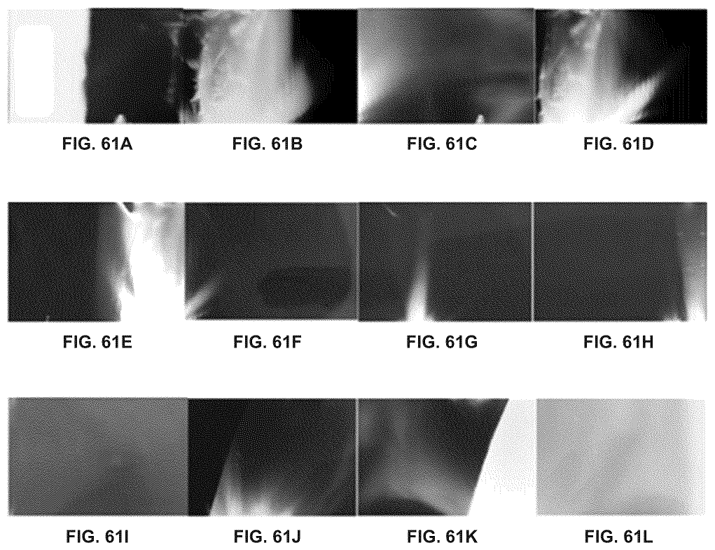

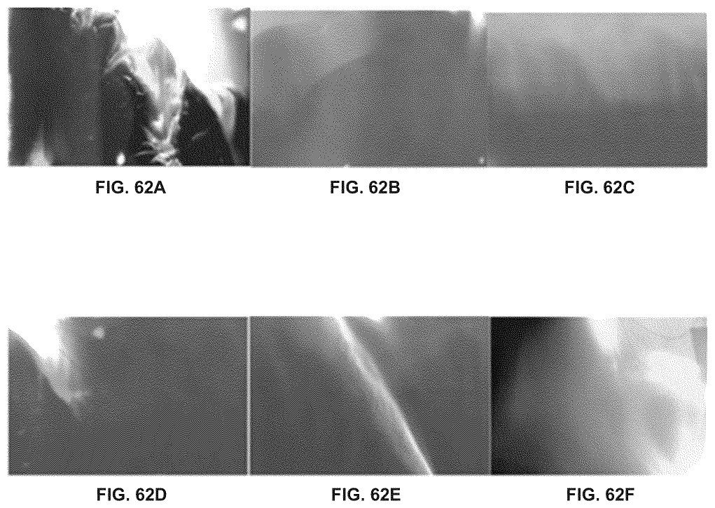

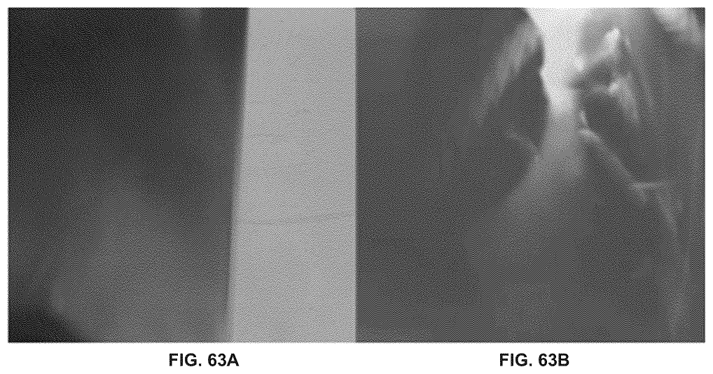

Twenty-eight farograms were obtained as a result of four tests: (Experiment No. 63, FIGS. 60A-H), his wife (Experiment No. 64, FIGS. 61A-L), their son (Experiment No. 65, FIGS. 62A-F), and their granddaughter (Experiment No. 66, FIG. 63A and FIG. 63B). They characterize those physical processes that occur during the formation of the composition that is being studied. The inventor has no right to work with viruses and send film with the virus to the laboratory. Farogram quality is good. They were obtained with a film length of 12 to 2 farograms, but it is impossible to detect the virus if it is absent. FIG. 63B illustrates some fragments in the nose of the inventor's granddaughter, similar to how they were found in his body (see FIGS. 43A-C). When protons remove electrons from K—or L-levels, p-photons are emitted due to the transition to these levels of both free and electrons from higher levels. Such radiation, called cascade radiation, is observed when materials are irradiated with high-energy X-ray photons and gamma rays. Modern science has accumulated extensive experience studying cascade radiation [See: A. Moves, R. G. Wilks, A. G. Kochur, E. Z. Kurmaev, Resonantly excited cascade X-ray radiation La, Phys. Rev. B (2005) 72, 075129]; Raman spectroscopy using a laser has been widely used in nanotechnology, biology, and pharmacology [See: H. Butler, L. Ashton, B. Bird, et al., Using Raman spectroscopy to characterize biological materials. Nature Protocols V. 11, 664-687 (2016). https://doi.org/10.1038/nprot.2016.036], which can be used to reveal the farogram code.

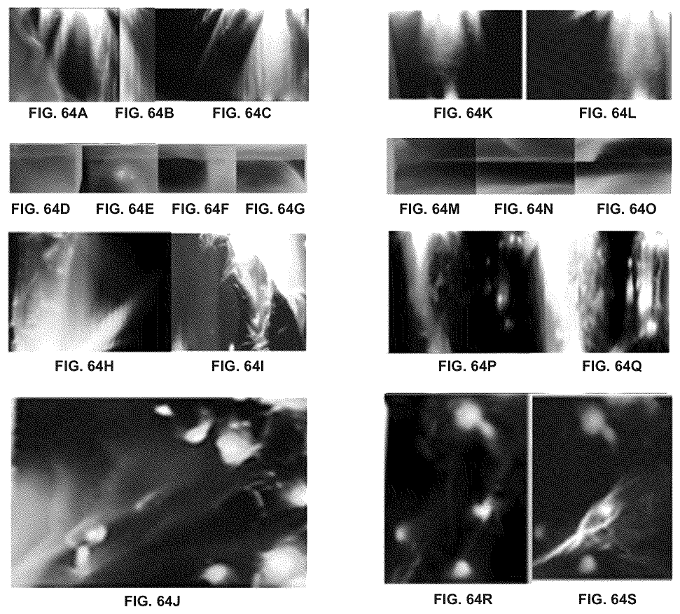

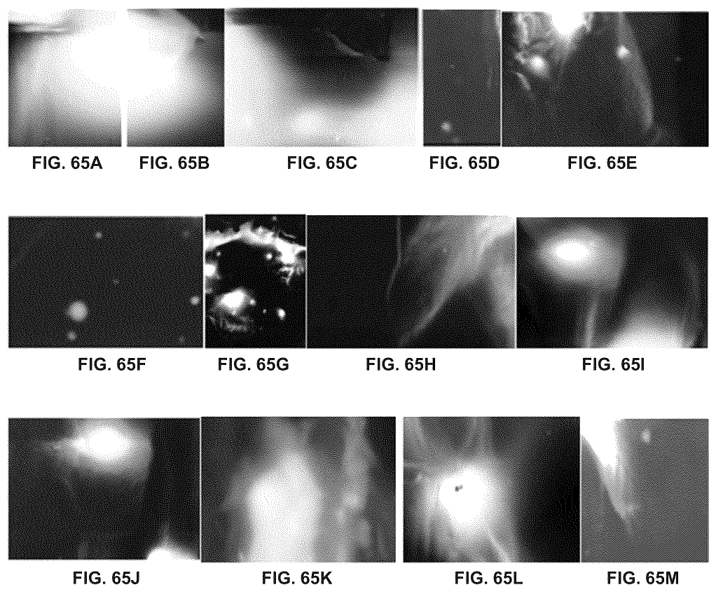

### Notes to FIGS. 64A-S and FIGS. 65A-M

OR=organic, NO=inorganic.

The fourth OR farogram, shown in FIG. 65G illustrates the right clavicle of the inventor but is not described in the text. This farogram is obtained from studying the inventor's right collarbone, showing processes whose mechanism remains mysterious.

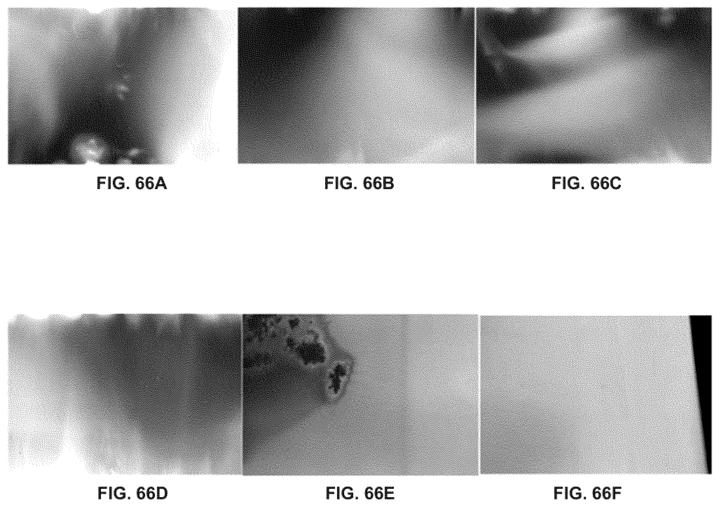

Experiment No. 67, FIGS. 66A-F. The experiment was carried out on PORTRA 800 Kodak professional film. Six farograms illustrate the p-radiation of a test similar to the test for sugar content in the body. A swab that absorbed a drop of the inventor's blood was placed on the film's surface in the center and exposed for an hour. Farogram a illustrates BL and water synthesis. The synthesis of water is observed on the farogram shown in FIG. 66E; a clear boundary between the dark and color areas is observed on the farogram shown in FIG. 66F. The red spectral line is observed on the farogram shown in FIG. 66E. The given farograms require special studies to use the information obtained in the experiment.

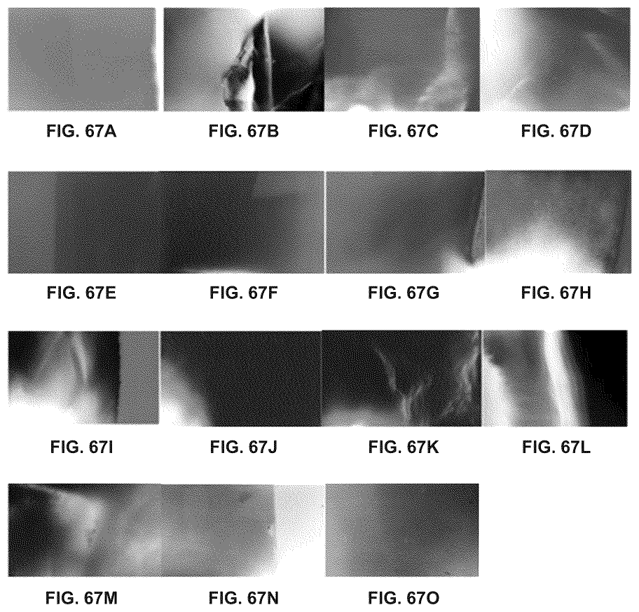

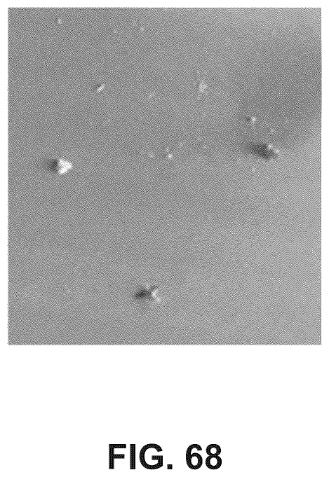

Experiment No. 68, FIGS. 67A-O. Fifteen farograms illustrate the result of a second test similar to the inventor's blood sugar test. A swab with two drops of blood was located where farograms shown in FIG. 67C and FIG. 67D were obtained. Thus, ten farograms were formed, as shown in FIGS. 67I-O, on the right and two on the left, shown in FIG. 67A and FIG. 67B, from the drops of blood. All farograms are interesting, but we note only those that have not been observed before: FIG. 67B, FIG. 67F, and FIG. 67I. Of particular interest is FIG. 67N. At least a portion of FIG. 67N is shown in FIG. 68. We observe unknown fragments located in the same direction, possibly in the direction of blood flow.

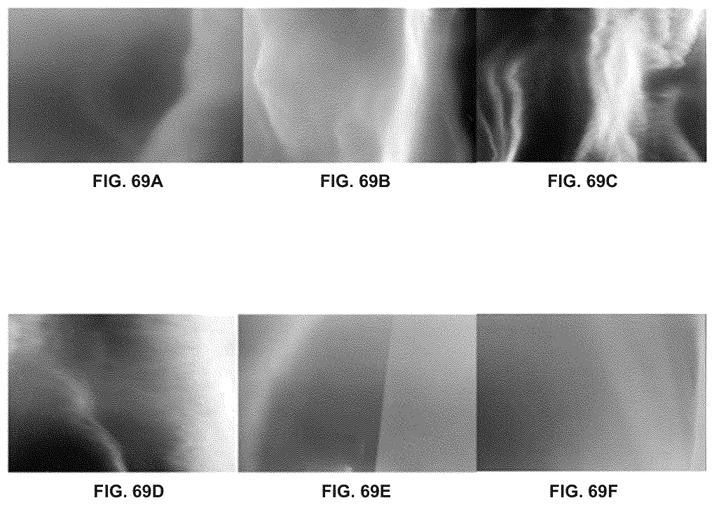

Experiment No. 69, FIGS. 69A-F. Six farograms illustrate the p-radiation of a sample similar to a urine test. The moistened tampon was placed on the film's surface in the farograms shown in FIG. 69B and FIG. 69C and kept for an hour. The maximum intensity of p-radiation is observed under the tampon. The yellow stripe on the farogram shown in FIG. 69B and the clear border on the farogram shown in FIG. 69E is particularly interesting.

The similarity of farograms demonstrating the processes in organic and inorganic molecules is shown in FIGS. 64A-S and FIGS. 65A-M indicates that the nature of the processes in inorganic and organic materials is electromagnetic and is accompanied by p-radiation. Still, such a coincidence is observed only on one of the farograms or its part. Secondly, the radiation frequency can be close but different. Modern experimental methods make it possible to measure the wavelength with an accuracy of a thousandth nanometer. FIGS. 64A-J show farograms that reveal processes in organic materials. FIGS. 64K-S show farograms that reveal processes in inorganic materials. FIG. 65A, FIG. 65B, FIG. 65F, FIG. 65H, and FIGS. 65I-L show farograms that reveal processes in inorganic materials. FIGS. 65C-E and FIG. 65G show farograms that reveal processes in organic materials.

Farograms make it possible to compare human behavior with the behavior of animals and insects and the results obtained from the study of technical devices that replace it. It is known that muscle contractions' efficiency exceeds modern devices' efficiency. The mechanics of muscle contraction is revealed by analyzing the farograms of the muscle and brain and comparing them with the farogram of a mechanical device. The inventor experimentally proved the possibility of simultaneously obtaining farograms in different directions. The total energy of p-radiation is determined using the integrating Ulbricht sphere.

It is known that the nature and mechanism of muscle contractions, brain memory, and sense of smell remain unresolved problems for biology. Understanding the mechanism of the collective behavior of a flock of birds, insects, schools of fish, ants, or bees is essential to their use. The use of p-learning helps in solving these problems. For example, brain memory is examined using farograms like those shown in FIGS. 41A-H, FIGS. 44A-M, FIGS. 47A-I, FIGS. 48A-F, and FIGS. 49A-I. The study of the behavior of a school of fish is significant for breeding fish for their catch. Understanding the signals that control a swarm of locusts will allow them to be directed to the surface of a reservoir or swamp to protect plants. There is no doubt that electromagnetic waves give such signals. The sense of smell is due to the effect of a chemical compound on the cells of a living organism, which is accompanied by p-radiation.

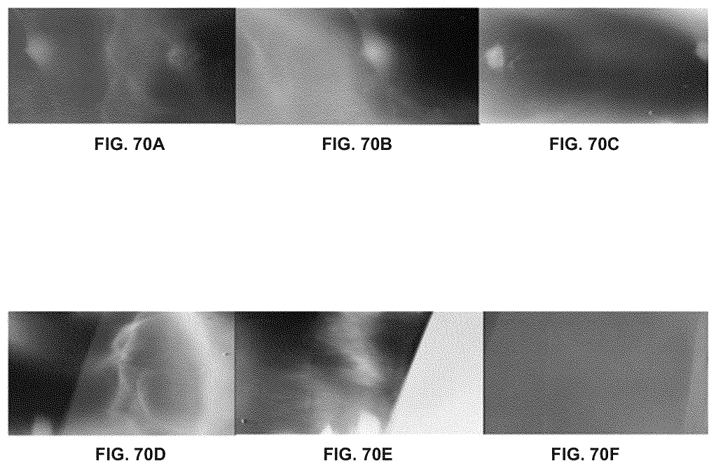

Experiment No. 70, FIGS. 70A-70F. FIGS. 70A-70F illustrate farograms obtained from the emission of p-photons from two drops of mineral water falling from a height of one meter onto the surface of a paper container where KODAK 800 film was placed-exposure 4 minutes. The drops fell in an area of the farogram shown in FIG. 70C. It is possible that a “quantum mirage” is observed on the farograms shown in FIG. 70A and FIG. 70B. A clear, flat border between the dark and colored areas is observed on the farograms shown in FIGS. 70D-F.

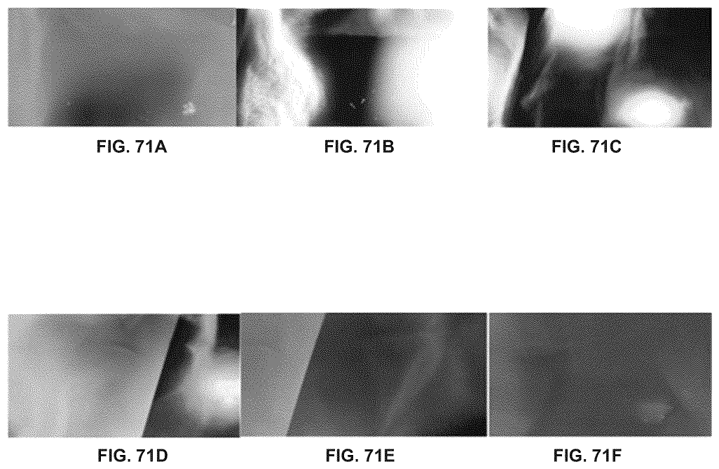

Experiment No. 71, FIGS. 71A-F. A drop of an alcoholic iodine solution fell on a container with photographic film, similar to the previous experiment. But instead of one drop image, three BLs are observed. Let us pay attention to the fact that similar figures are repeated in various experiments with other compositions and the nature of the experiment. For example, we see identical farograms in FIG. 14D, FIG. 15B, FIG. 15C, FIG. 28D, and FIG. 28H. The farograms shown in FIGS. 71C-E depict a flat border separating dark and colored areas.

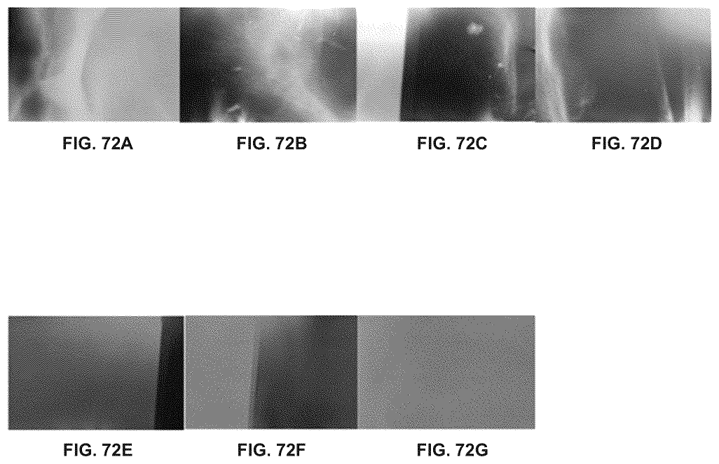

Experiment No. 72, FIGS. 72A-G. A drop of vitamin E oil fell on the surface of the container from a height of 2 cm in the region of the farogram shown in FIG. 72C, but there are three BLs in the dark region on the farogram. A flat border separating the dark area and the color area is observed on the farograms shown in FIG. 72C, FIG. 72E, and FIG. 72F.

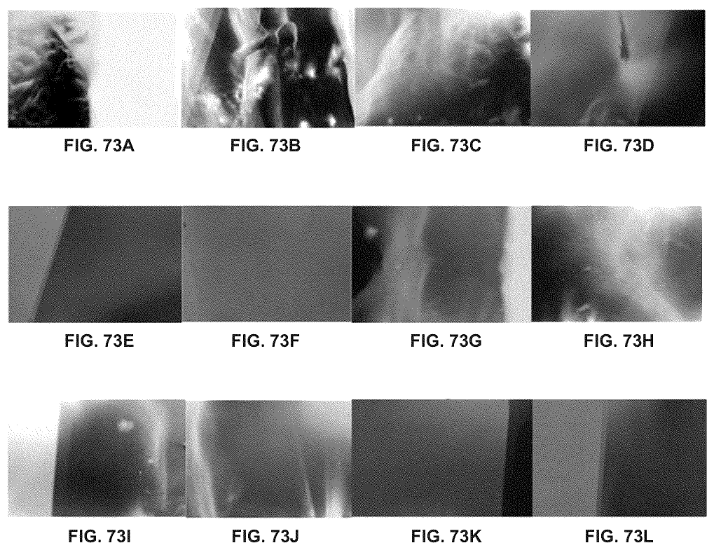

Experiment 73, FIGS. 73A-L. A thin layer of La Roche-Posay® Lipikar® AP+M Triple Repair Moisturizing Cream was applied to the surface of the container in the form of a strip 10×4 mm². In this experiment, twelve farograms must be divided into two parts. The first part includes seven farograms, shown in FIGS. 73A-F, and the second five, as shown in FIGS. 73H-L. It is easy to see that the second part, as shown in FIGS. 73H-L, consists of the five farograms shown in FIGS. 72B-F.

It is necessary to pay attention to this coincidence of the farograms of FIGS. 72B-F and FIGS. 73H-L, indicating the identity of the content of inorganic substances.

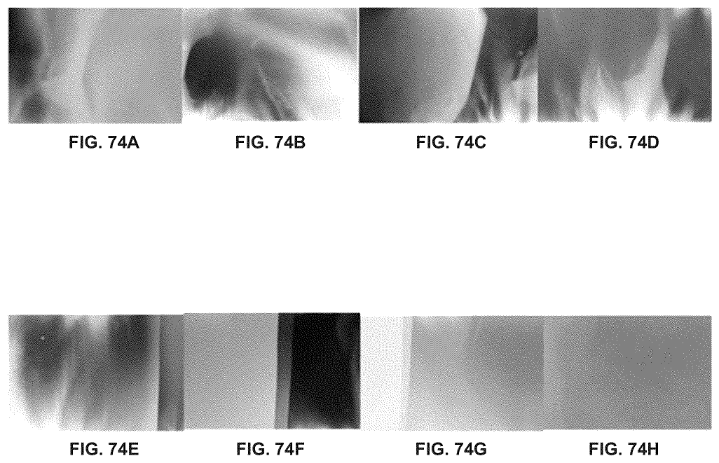

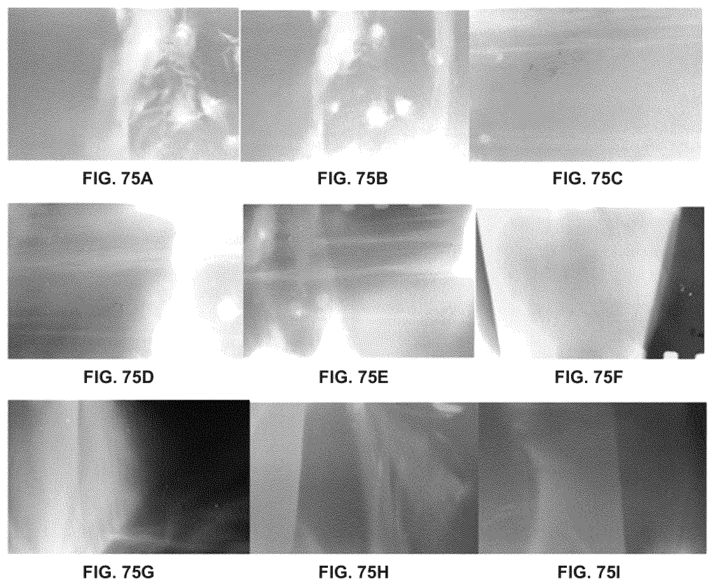

Experiment 74, FIGS. 74A-H. A drop of omega-3 from fish was carefully applied to the surface of the container. Eight frames illustrate various types of farograms: water synthesis (as shown in FIGS. 74B-G), a flat border spread out (as shown in FIGS. 74E-G), and an image in FIG. 75A similar to that shown in FIG. 72A.

The inventor, who had type 2 diabetes, used the FreeStyleLibre 2 and conducted a study with positive results on lifestyle changes and improvements to the device. The device contains a computer program that allows you to create a menu to maintain glucose levels in the proposed range and quickly return to this range by supplementing your diet with physical activity. Intense p-radiation makes it possible to create sensors for measurements based on farograms of glucose levels and the state of the pancreas. The disadvantage of the device is that the sensor must be immersed in the body and replaced after two weeks of power failure. The use of farograms allows you to eliminate these shortcomings.

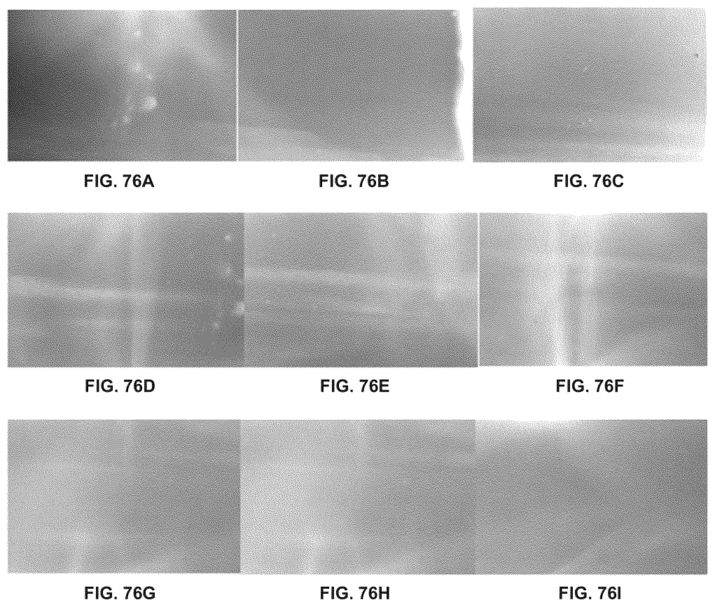

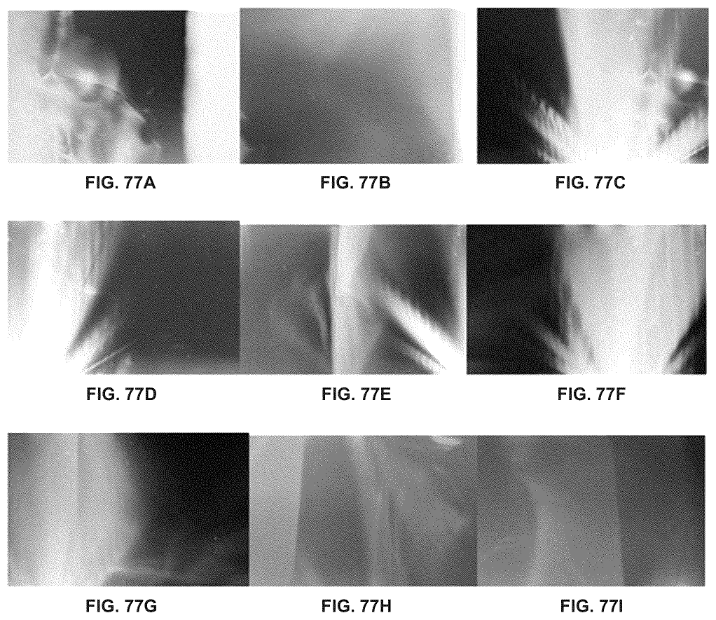

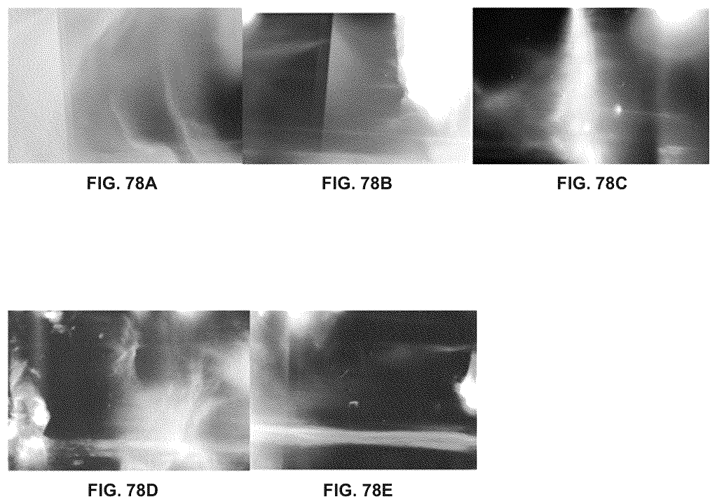

Experiment 75, FIGS. 75A-I, FIGS. 76A-I, FIGS. 77A-I, FIGS. 78A-E. The nine farograms shown in FIGS. 75A-I demonstrate the p-radiation of the device's sensor. This means that a sensor without a battery can be created. FIGS. 76A-I show the emission of the device when displaying information. Such farograms are observed for the first time. Two more photographic films were used simultaneously with them. The third Film was placed on the head of the inventor to obtain farograms of the brain, while the fourth was placed on the forearm next to the sensor.

Two more photographic films were used simultaneously with them. The third film was placed on the head of the inventor to obtain farograms of the brain, while the fourth was placed on the forearm next to the sensor. The farograms of the organs of the inventor and members of his family are shown repeatedly in this description. The four farograms of shown in FIGS. 77C-F show the particularly intense p-radiation due to water synthesis, which was observed when examining the chest of the inventor, as shown in FIG. 43F, FIG. 43H, and FIG. 43I.

An increase in brain water synthesis indicates an increase in energy, which should be considered a response to an increase in glucose followed by a decrease in glucose. This fact shows that the brain not only records changes in the organs, but also controls them to eliminate the pathology that has arisen in the pancreas. It is known that glucose levels depend on nutrition, the combustion of which can improve the functioning of the pancreas or worsen it so much that a pathology occurs that disrupts the functioning of another organ, for example, the foot, to such a level that wounds occur.

The organ farogram provides the most accurate information about the energy processes in it at the time of examination. The ease of obtaining farograms and the possibility of obtaining them independently of other studies makes it possible to detect pathology at an early stage of its occurrence. Of particular importance is the analysis of pharograms of the organ and brain obtained simultaneously. An effective method for treating wounds caused by diabetes is proposed in the work [Advanced Materials (2023). DOI: 10.1002/adma.202304638], but information about changes in the functioning of the pancreas is especially important.

It should be noted that the longitudinal red lines are preserved in the five farograms presented in FIGS. 78A-E. It is known that pancreatic cancer is most often detected when surgery is no longer possible.

Tumors, especially malignant ones, are one of the most pressing problems in medicine. The mechanism of their origin, development, and spread is often unclear. Pancreatic cancer is often discovered when surgery is no longer possible. The number of identical farograms is small for statistical analysis. However, these isolated cases provide confidence that searching for patterns using farograms is more effective than other methods for the early detection of tumors. The simultaneous analysis of farograms of the brain with the organ being studied is of particular importance. The experiment lasted eight weeks. Monitoring blood sugar levels after an hour helped create a dietary menu to keep glucose levels below 160 mg/dL and study the recovery time when it dropped to 165 mg/dL. If at the beginning of the experiment with a strict diet, the average content per week was 148 mg/dl in the range of 120-198 mg/dl, recovery time was 3 hours, then at the end, without following a diet, the average value was 136 mg/dl in the range of 107-182 mg/dL, recovery time 50-90 minutes. But the main conclusion of the experiment is that with the help of farograms, it is possible to establish the cause of diabetes, allowing humanity to get rid of it.

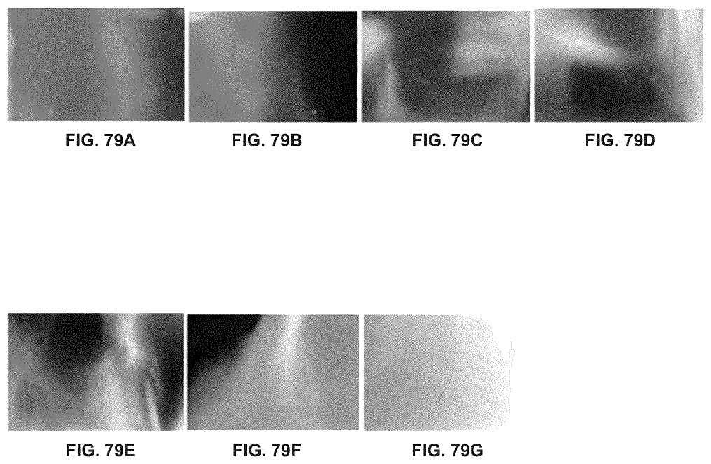

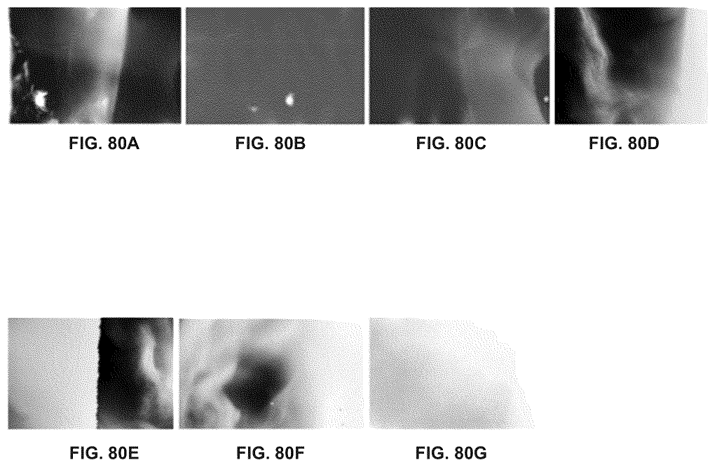

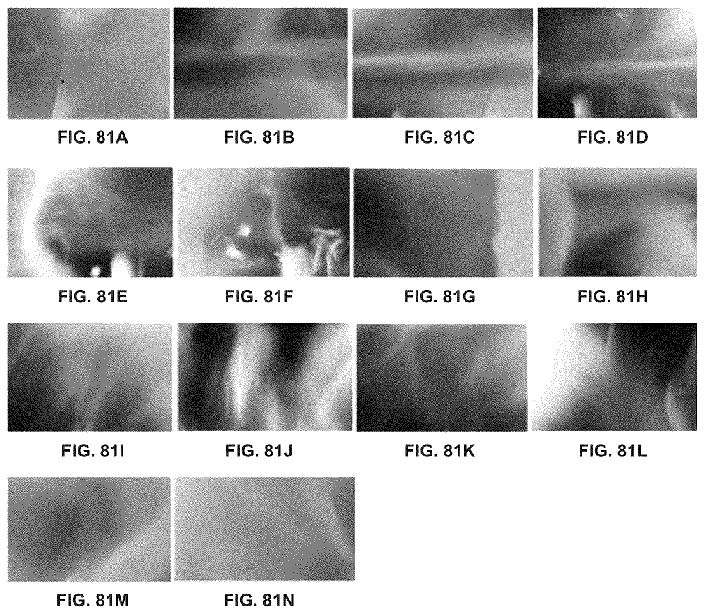

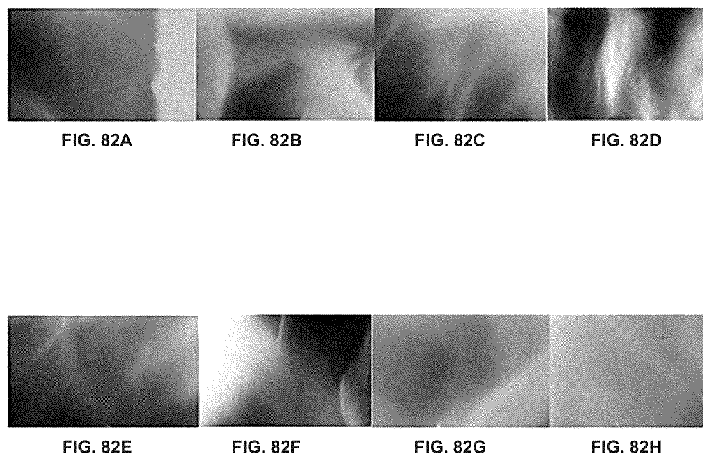

Experiment 76, FIGS. 79A-G, FIGS. 80A-G, FIGS. 81A-N, and FIGS. 82A-H. The unique role of the brain in human life is well known but attempts to prove and use this hypothesis continue [See https://jamanetwork.com/searchresults?author=Yoni+K.+Ashar&q=Yoni+K.+Asharl. Reattribution to Mind-Brain Processes and Recovery From Chronic Back, JAMA Network Open. 2023; 6(9):e2333846. doi:10.1001]“To test whether the reattribution of pain to mind or brain processes was associated with pain relief in pain reprocessing therapy (PRT) and to validate natural language-based tools for measuring patients' symptom attributions.”

Meanwhile, perhaps for the first time, with the help of farograms, experimental evidence of brain control over processes in the organ was demonstrated. It gives us confidence that evidence of brain control over organs can be obtained.

Four films were applied to the body of the inventor's son as follows. Film No. 1 was located to the left of the heart from top to bottom; Film No. 2 was located parallel to it on the right; Film No. 3 was located parallel to them against the heart, but behind; Film No. 4 was positioned perpendicular to the head at forehead level.

The experiment was conducted on a treadmill for 10 minutes at a 2-2.5 mile/hour speed.

The farograms obtained from the experiment are shown accordingly: No. 1-FIGS. 79A-G, No. 2-FIGS. 80A-G, No. 3-FIGS. 81A-N, No. 4—FIGS. 82A-H.

The result of the experiment is of particular importance. If FIGS. 73A-L confirms that Triple Repair moisturizer contains vitamin E, then FIGS. 81A-N and FIGS. 82A-H confirm the unique role of the brain, which is the response to any process in every associated body organ.

Eight matches between farograms of the heart, FIGS. 81G-N with farograms of the brain FIGS. 82A-H exclude randomness and is sufficient to prove the existence of the law by which it is conditioned. The difficulty of detecting such a coincidence in farograms is because, firstly, the brain controls all organs simultaneously. Secondly, farograms of one studied area are displayed spatially, and to detect a coincidence, it is necessary to conduct multi-path studies. This means the brain's functions are so unique that they cannot be compared to a computer.

Thirdly, the simultaneous study of processes in the body using modern high-precision and sensitive methods and the farogram method increases the efficiency of the study, in which pathology, including the occurrence of a tumor, will be detected at an earlier stage.

No. 2—FIGS. 80A-G, No. 3—FIGS. 81A-N, No. 4—FIGS. 82A-H.

The result of the experiment is of particular importance. If the farograms shown in FIGS. 73A-L confirm that the Triple Repair Moisturizer cream contains vitamin E, then the farograms shown in FIGS. 81A-N confirms the unique role of the brain, which is the response to any process in each organ of the body associated with it.

This conclusion is confirmed eightfold, for the eight farograms of the heart as shown in FIGS. 81G-N are identical to the eight farograms of the brain as shown in FIGS. 82A-H.

This means the brain's functions are so unique that the computer cannot be compared.

Secondly, the simultaneous study of processes in the body using modern high-precision and susceptible methods and the farogram method increases the efficiency of organ control, in which pathology, including the onset of a tumor, will be detected at an earlier stage.

Such several coincidences testify to a law by which they are conditioned. However, the number of farograms in space is so large that only a tiny part can be compared during the experiment. Thus, such a law is essential to eliminate the static probability. The inventor experimented to confirm this feature of farograms.

Experiment No. 77 was carried out similarly to the previous one. Still, two films were used, one for studying brain radiation, and the second was located like film No. 3 in the previous experiment.

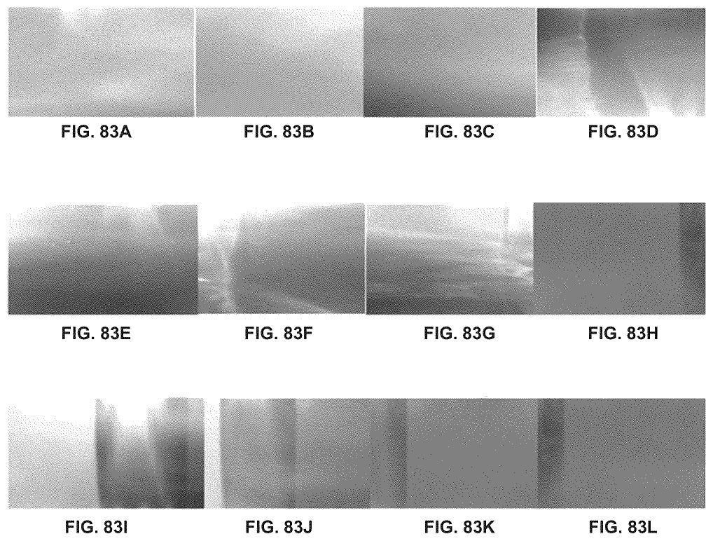

Experiment No. 83, FIGS. 83A-L shows farograms obtained by emitting p-photons from the brain of the inventor's son, similar to the previous experiments, but for four minutes. Let's carefully analyze all 12 farograms using slight contrast. Farograms demonstrate all the features of the p-radiation of organs. 1) Fan-shaped synthesis of water: FIG. 83A, FIG. 83D, and FIG. 83J; 2) a flat border between dark and colored areas: FIG. 83D, FIG. 83N, FIG. 83J, FIG. 83K, and FIG. 83L; 3) Parabolic boundary of dark and colored areas: FIG. 83D, FIG. 83F, FIG. 831, FIG. 83G; 4) Water synthesis in the dark area: FIG. 831 and FIG. 83J; 5) luminous ball (BL): FIG. 83B, FIG. 83E, and FIG. 83L; 6) Side radiation: FIG. 83D, FIG. 83F, FIG. 83G. Farograms demonstrate the process, indicating that a seemingly neutral molecule, evaporating from the surface and wetting the paper, emits many photons in various combinations unique to it. The farograms shown in FIG. 83I and FIG. 83J, characterizing the synthesis of water, are located on the dark background. This transformation is observed twice in other experiments, for example, as shown in FIG. 43F with a flat boundary and FIG. 43I with a parabolic one. Bright red-white and yellow-white BL flashes are observed repeatedly on both colored and black backgrounds.

Light is emitted when an electron in an atom moves from a higher energy level to a lower one or recombines. Therefore, the atom has been ionized or excited; for example, an ionized hydrogen molecule was formed. But comparing organ cell farograms with the results of many years of research is not productive since they were performed not on living cells but on dead ones. These photographs are black and white. The most critical parameter, the frequency of the atomic reaction of life, is not used. [See. www.uni-mainz.de Mitochondria Dr. Jastrow's EM-Atlase].

The effectiveness of using farograms depends on the degree of their decoding. The inventor postulates that the formation of a well-defined flat, parabolic, or blurred boundary between dark and colored areas observed with p-radiation of inorganic materials and cells is due to the formation of hydrogen peroxide, similar to what was demonstrated by Faraday in the synthesis of water. The color and shape of the luminous areas are different, but one-color or two-color spheroidal formations similar to ball lightning are often observed. But it must be remembered that all organ processes occur at body temperature. The inventor assumes that quasi-spherical regions are formed due to the formation of ions of hydrogen molecules, the emission of which occurs due to recombination with electrons.

Mental illnesses are entirely caused by brain pathology and are challenging to treat due to the lack of effective non-invasive analysis methods. One of the causes of mental illness is stress. Two articles published by the staff of world-famous laboratories demonstrate the problems of psychiatry. An analysis of these works shows that the behavior of mice and extrapolation of the result to human behavior remained the only research method until 2023. [See: Lopez et al., Single-cell molecular profiling of all three components of the HPA axis reveals adrenal ABCB1 as a regulator of stress adaptation, Sci. Adv. 2021; 7: 4497 27 Jan. 2021] and [E. Brivio et al., Sex shapes cell-type-specific transcriptional signatures of stress exposure in the mouse hypothalamus, Cell Reports (2023). DOI: 10.1016/j.celrep.2023.112874]

“E. Brivio et al. generated a rich single-cell RNA sequencing dataset of the mouse paraventricular nucleus of the hypothalamus (PVN) to show that the response to acute stress is encoded differently in cell types and sexes. This approach identified oligodendrocytes as cells susceptible to stress”. “Stress-related psychiatric disorders and the stress system show prominent differences between males and females, as well as strongly divergent transcriptional changes. Despite several proposed mechanisms, we still do not understand the molecular processes. Here, we explore the contribution of cell types to transcriptional sex dimorphism using single-cell RNA sequencing.” “Altogether, our results indicate the need for more single-cell resolution studies in females and males and highlight directions to dissect the molecular processes driving sex differences in normal physiology and stress response. When combined with other recent studies, our results provide a further molecular and cellular characterization of the hypothalamus and identify several cell types as priorities in which to explore sex differences in the context of stress in the human brain, which could provide a new understanding of the origin of the sex differences in brains.”

P-radiation makes it possible to study any organ without disturbing its functions, including remotely. The human brain is so sensitive to external influences that it reacts to individual photons. This fact is confirmed by experiments on the study of radioactivity performed by students of E. Rutheford and luminescence by S. I. Vavilov. Therefore, research into brain function using implants damages the brain and should be prohibited, but it is carried out even when it is not necessary, for example.

1* “A research team at Purdue University recently introduced a new approach to enable communication between the human brain and computers via wireless neural implants. Their proposed approach, outlined in Nature Electronics, relies on a two-phase process that slowly unfolds in the brain, allows a small sensor implanted in the brain to sense and transfer information to a wearable headphone-shaped device, without disrupting the human body's physiological processes.”

2* “This technology, when combined with further advancements in deep-brain wireless power transfer, would make it possible to gain fundamental insights into disorders like Parkinson's disease, Tourette Syndrome, Epilepsy, Depression, Anxiety, and obsessive-compulsive disorder.”

[See Baibhab Chatterjee et al., Biphasic quasistatic brain communication for energy-efficient wireless neural implants, Nature Electronics (2023). DOI: 10.1038/s41928-023-01000-3].

Experimental confirmation of the generally accepted hypothesis that the human brain is nature's highest creation allows us to conclude that the atomic-proton reaction is the radiation of living nature.

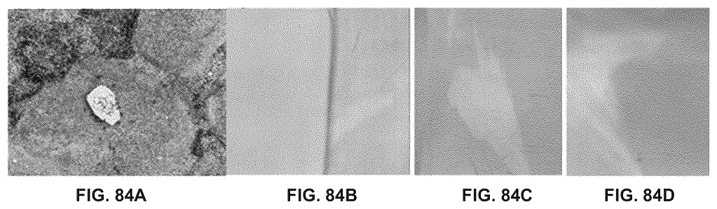

Experiment No. 84, FIGS. 84A-D. Black ants, whose length does not exceed 2 mm, have created an anthill on a plot of land covered with ceramic tiles. The entrance to it was located in the gap between the tiles. The experiment was carried out after sunset to exclude the effect of solar radiation on the photographic material. A lone ant, which was 80 cm from the entrance to the anthill, was fenced off from it with a pillow filled with foam, the thickness of which was 10 cm after it was tightly pressed against the tiles. This ruled out the possibility of good communication through a pinnacle on a path of 50 cm. Next, a boiled yolk was laid out in front of the ant on the shell of a chicken egg. Forty-six seconds after the lone ant tried the bait, an ant appeared from the anthill and headed toward the bait. The sound absorber was removed, and the ants, overcoming the distance, carried the bait, as seen in photos shown as FIGS. 84A-D. The gap between the tiles forming the chute was filled with crushed yolk and covered with a Film. The ants worked until sunrise. Eleven frames contained the green line in the farogram shown in FIG. 84B and ten black and white farograms like those shown in FIG. 84C and FIG. 84D. The experiment allows us to conclude that the remote communication between ants uses electromagnetic waves. The absence of colored farograms is due to low energy losses. The conducted experiment is a demonstration and requires repetition.

Experiment No. 85. The green fly entered the garage 76 seconds after beginning cleaning the fish, which was carried out at a distance of four meters from the entrance, whereas the flies had not previously entered the garage. Two minutes later, three more flies arrived. The sense of smell is due to the action of substance molecules on receptors. The distribution of such molecules occurs due to diffusion. The diffusion rate is so slow that the molecules emitted by the fish do not reach the olfactory organs of the flies.

Consequently, flies capture p-photons emitted by molecules. The experiment shows that technical devices that exceed the sensitivity of animals or insects can be created using a set of frequencies of p-radiation, which is perceived as odor. This set of frequencies (process code) is encrypted in the farogram. Only the exact correspondence of the color farogram to the frequency spectrum makes it possible to use p-radiation as a sensor of olfactory radiation practically.

### Ways of Implementation of the Method

Implementing the method according to the invention requires preliminary determination of the measured and calculated physical parameters to obtain the desired result based on the decision made. The processes are then analyzed based on the farograms.

The implementation of the method is based on measuring the intensity of p-radiation at each frequency, monitoring, analyzing, and storing information. The frequency of p-radiation characterizes the atomic proto-reaction, which determines the emergence of an object and its change or decay. Thus, the research objects can be both materials and organs of living organisms. This means there is sufficient evidence for abandoning modern methods of controlling processes in inorganic materials and devices based on classical mechanics, which are refuted by experiments. Instead, advances in modern medicine are driven by diagnostic and treatment methods based on electromagnetic radiation. Information about the condition of an organ that can be obtained using a farogram is many times greater than the information obtained using X-rays and complements the information obtained using MRI. P-radiation is non-invasive, does not require complex equipment, and can be used repeatedly for diagnosis and treatment.

However, the information in the color farogram is mostly encoded and requires interpretation of the mechanism of its conditioning. However, the main parameter that can be used to prevent a man-made disaster and warn of an earthquake earlier than using seismic methods is the energy accumulated in the destruction zones. P radiation is electromagnetic. It spreads in the atmosphere, liquids, glass, plastics, and even alloys and composites. Inorganic materials can be used simultaneously with X-rays and synchrotron radiation.

But n-photons are non-invasive; they do not break the bonds between atoms. The bond is broken by photons caused by the transition of electrons from the upper level to the lower one. But the simultaneous action of many such photons is necessary to damage a solid body, form a crack, or destroy it. The problem of preventing catastrophic damage is described in the author's U.S. Pat. No. 11,047,813; p-photons indicate where such a reaction occurs and what chemical element emits these photons. It is possible that the rate of energy accumulation can be determined from the intensity of n-radiation, but this needs to be proven experimentally.

Therefore, all devices designed to study the electromagnetic spectrum can be used together in a new branch of technology called protonics, using photonics methods and equipment.

### Examples of Application of Farograms

Farograms in the invention illustrate the effect of p-radiation on a candle flame or direct effect on photographic film. However, the inventor deciphered the information only for the synthesis of water, the formation of cracks, and the appearance of living organisms. A hypothesis about the mechanism of formation of other types of farograms, based on intuition, needs experimental verification. Therefore, to decipher all the information, it is necessary to compare it with other methods.

1. M. Faraday experimentally proved the synthesis of water in wax and metals during their combustion. B. V. Novozhilov was the editor of one of the translations of Faraday's book into Russian. He gave it a second name: “Burn to live.” This interpretation of Faraday's sixth lecture by a nuclear physicist should be attributed to the combustion of hydrogen (water synthesis), as the inventor did.

Cox [See, H. Cox on Impact on Elastic Beams. (1849) Cambridge Philosophical Society V. 9, 73-78]proposed a method for investigating the deflection of a railway bridge based on the measurement of physical parameters. His empirical equation made it possible to experiment with an error of 0.13 to 3%. Modern methods for measuring physical parameters are many times higher than the accuracy and sensitivity of the method used by Cox. There is no obstacle to using the Cox method based on p-radiation instead of modern methods based on stress intensity factors.

The finite element method is used to solve strength problems, including in the design of aircraft, missiles, and ships, and analysis of the causes of catastrophes of structures, for example, bridges. But it is mathematical, and physical parameters are not used in it.

The photographs obtained from the experiment demonstrate the physical phenomenon that causes them. The p-photon absorbed by the photographic emulsion causes a photochemical reaction, enhanced during further processing with chemical reagents. This made it possible to understand the destruction mechanism, but it is not enough to estimate the energy of destruction caused by the p-photons of all absorbed frequencies. However, a photographic farogram exhibits bright radiation at a particular frequency. Therefore, photons emitted by some chemical element or compound do not form cracks. Modern experimental technology has sensors for detecting electromagnetic signals in the frequency range in which p-photons are emitted and computer programs for their analysis, storage, and transmission. A digital farogram is a real-time data table on radiation intensity at a selected frequency for a selected point with coordinates x₁y₁. For example, the relationship between the p-radiation energy E and the intensity I of spontaneous radiation is expressed by Eq.

$${E}_{n} = {α}_{n} {I}_{n} ( {v}_{n} , {t}_{n} ) {E}_{n} = {α}_{n} {I}_{n} ( {v}_{n} , {t}_{n} ) . \qquad ( 5 )$$

Here, the subscript n indicates the chemical element or compound, the radiation being investigated, and α is the coefficient of proportionality. The fundamental difference of the farogram is that the frequency of the p-radiation allows you to determine the chemical element by which the photon is emitted.

For example, we are investigating a Fe—Mn—C alloy synthesized in the atmosphere using ultrapure components. A farogram will illustrate the synthesis of water, even though hydrogen and oxygen are not part of the composition. Thus, it is necessary to investigate those elements used for synthesis and those numerous phase transitions that occur in the Fe—C system to find the chemical element whose atoms form a destruction domain. It is necessary to obtain data according to equations

$${E}_{Mn 1} = {α}_{Mn 1} {I}_{Mn 1} ( {v}_{Mn} , {t}_{1} ) \qquad ( 6 )$$

$$and$$

$${E}_{Mn 1} = {α}_{Mn 1} {I}_{Mn 1} ( {v}_{Mn} , {t}_{2} ) . \qquad ( 7 )$$

Several points obtained before the crack forms allow the creation of a device for investigation.

The farogram method makes it possible to investigate the processes of crack formation, sequentially controlling all its stages using photographic and digital programs. The time interval is not limited by an upper or a lower value, but external influences can increase the radiation intensity. A change in a is possible, as shown in equation

$${E}_{Mn 2} = {α}_{Mn 2} {I}_{Mn 2} ( {v}_{Mn} , {t}_{2} ) . \qquad ( 8 )$$

Thus, the function E=F(t) analysis makes it possible to solve the set problem in a laboratory experiment and create a device for non-invasive control of those elements that form a fracture domain in alloys. Laboratory studies are performed to assess the energy limit at which a crack forms. Knowing the element that causes the formation of the crack, the researcher decides on possible changes in technology, guaranteed time of use and replacement, strengthening control over this element.

Iron and carbon are special in technology, plants, and living organisms. M. Faraday noted in one of his lectures that carbon behaves like no other material. At that time, two phases were known: graphite and diamond. Today, fullerene, nanotubes, and graphene are known, and other local formations are observed in iron and carbon alloys. However, the breakdown of phase transitions, for example, residual austenite-martensite, remains unresolved until now, despite many experimental studies, since the characteristic feature of the atomic interaction of graphene, which has one free bond, is not considered. Nevertheless, the presence of graphene in iron alloys up to one nanometer in size has been proven experimentally [See: K. Y. Wen, T. J. Marrow, and B. J. Marsden, The microstructure of nuclear graphite binders, Carbon, 2008, 46 (1), 62-71].

The fuselage of the Boeing 777X was destroyed by an explosion that followed before reaching its intended critical load during testing. The FAA agreed with the manufacturer that the hardening could be done based on the analysis of the cause of the failure and that it was put into service without retesting. The aircraft was manufactured and offered for use. This decision is based on several mistakes due to ignorance of destruction laws. First, the cause of the explosion and subsequent destruction, including the passenger door, is not elastic but electromagnetic energy. Second, aluminum is the critical load for the fuselage because the composite material containing carbon fundamentally differs from aluminum's properties. Thirdly, the computer program is erroneous because it is based on analyzing acoustic waves. At the same time, the propagation speed of the impulse leading to destruction is 50,000 times bigger, which excludes the crew's ability to make any decisions in flight. This means that operational safety is not guaranteed, which is a crime. In this case, the reliability of the Boeing 777X can be verified only by the non-invasive method proposed in this invention. The new computer program should be developed based on the farograms obtained by non-damaging bending of the material and the equations developed based on the work of Maxwell. Existing methods do not apply to this.

The method based on using farograms makes it possible to evaluate the reliability and durability of the screw, rivet, and adhesive fasteners at the production stage and during operation, including remotely. The non-invasive test lasts a fraction of a second without compromising the integrity of the product.

We restrict ourselves to the analysis of two experimental studies of the causes of crack formation in the riveted joint of the Boeing 727-232 fuselage, performed by the same method. The results of crack length measurements are given in the tables. Theoretical analysis of the study's results was carried out using the NASGRO equation. The inventor used tables to demonstrate his proposed method.

First, we will demonstrate the analysis based on one of the tables and photographs in [B. R. Mosigny, “Fatigue Damage Assessment of the Fuselage Structure of Aircraft in Service,” Thesis Presented to the Faculty of Drexel University, 2007], as shown on Rivet A40.

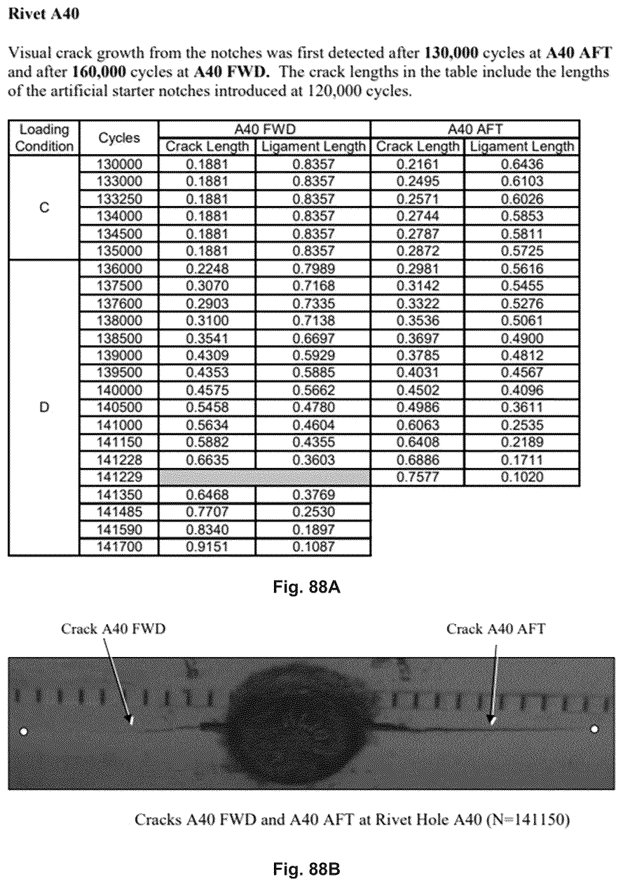

Let us note the features in the table, shown in FIG. 88A, which demonstrates the results of measuring the length of cracks on the left A40 FWD and right A40 AFT, shown in FIG. 88B. The first crack length measurement, performed after N=130,000 flexure cycles of the fuselage panel, showed that the crack on the right is longer than on the left. This difference remained until N=141228. Let us pay attention to the fact that the length of the crack on the left did not change during the first six tests.

Let's see how the data obtained based on the experiment are used from the position of quantum mechanics to ensure flight safety. The table allows us to demonstrate the application of the method proposed in U.S. Pat. No. 11,047,813 of the inventor.

We will use an equation to estimate the energy that is expended to break the bond during the formation of a crack

$$Δ {U}_{2} = \frac{h {ε}_{b}}{{a}^{2}} \cdot \frac{Δ l}{Δ N} . \qquad ( 9 )$$

Where h is the thickness of the metal, ε_{b} is the binding energy, a is the constant of the crystal lattice, Δl the lengthening of the crack with an increase in the number of cycles ΔN.

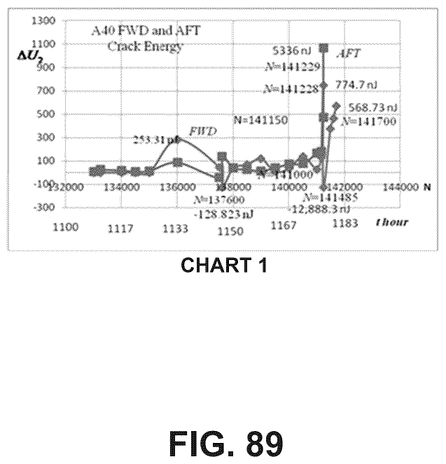

The graphs shown in Chart 1 of FIG. 89 illustrate the energy processes of the crack formation near the A40 rivet. Paying attention to the minimum energy increment −128.823 nJ at N=137600 is necessary. A similar minimum energy increment −12,888.3 nJ is observed at N=141485 (The scale changes a hundred times.)

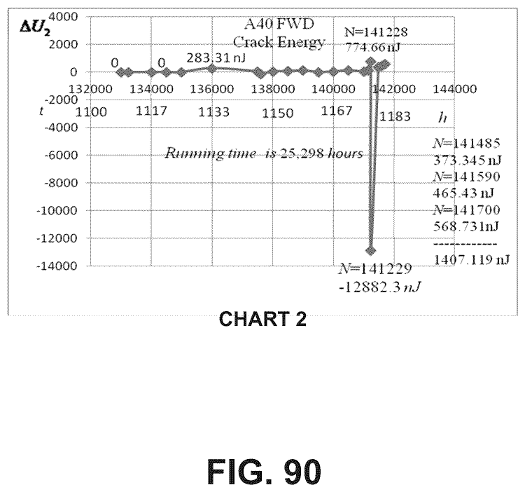

Chart 2 of FIG. 90 is presented in such a way as to highlight a deep low followed by three highs. But not all of the accumulated energy is emitted; there is still 11.475.18 nJ left, the stimulated emission of which will lead to the formation of a large crack. This is a blind (invisible) crack or a precursor of a crack leading to a catastrophe. [See, V. P. Rombakh, Nondestructive Monitoring of Atomic Reactions to Detect Precursors of Structural Failure, Singapore, 2023] Such accidents often occur so unexpectedly for the crew that their cause remains mysterious.

But there comes a moment when they all combine into one main crack, at which depressurization occurs, a catastrophe, and the death of passengers and crew.

However, as we know, in a riveted joint, cracks grow towards each other, practically between all rivets, remaining dangerous precursors.

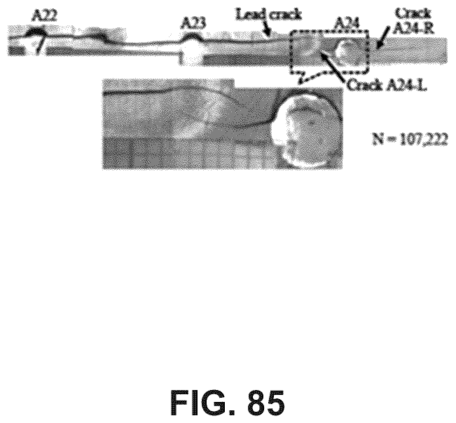

A study of cracks growing towards each other was carried out in [Abubaker Ali Ahmed, Initiation and Growth of Multiple-Site Damage in the Riveted Lap Joint of a Curved Stiffened Fuselage Panel: An Experimental and Analytical Study A Thesis Submitted to the Faculty of Drexel University, 2007]. A photograph, shown in FIG. 85, taken from the work shows a fragment of a Boeing 727-232 fuselage, in which a crack 75.80 inches long (1925 mm) formed during the study. The author noted the unusual location of the cracks upon meeting but did not comment on it.

But we see that the crack growing from rivet A24 to rivet A23 “avoids” a direct meeting and grows not in a straight line. Still, in an arc, heading to some point on the opposite crack, a crack growing from rivet A23 of the rivet-to-rivet A24 “avoids” the meeting and grows, heading to some point on the opposite crack.

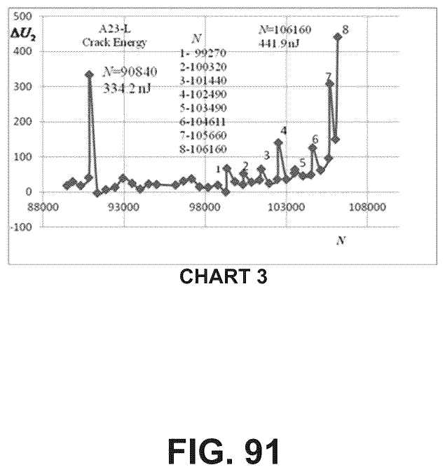

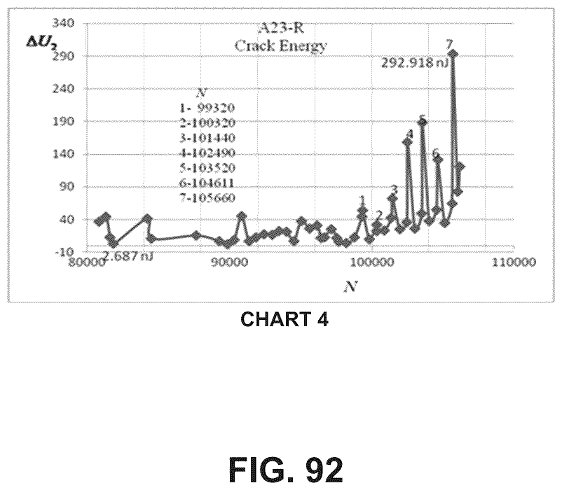

A crack with a length of 1925 mm was formed during the investigation, in which 106.217 cycles were performed. During the study, 4,508,920 tests were performed, which took 31,318 hours. At the same time, R. Mosigny's study lasted 25,298 hours. Chart 3, shown in FIG. 91, and Chart 4, shown in FIG. 92, illustrate how slowly energy can accumulate, but the bottom line is that any crack in the fuselage during flight can cause disaster.

The ease of obtaining farograms, reliability, and efficiency guarantees the possibility of immediate use of the proposed method.

The rejection of the use of quantum mechanics in the theories of strength and fracture led to the fact that USA PUBLIC LAW 100-591—Nov. 3, 1988 “RESEARCH PLAN AND REPORTS” (d) (1) has not yet been performed. This law was passed after the crash of a Boeing 737-292, which occurred on Jan. 28, 1988, during a flight. A crack 5.6 m long destroyed the fuselage, so the first three rows remained open. One passenger who was not wearing a seatbelt was killed. Nevertheless, the plane managed to land. The law obliges the creation of new non-destructive methods for monitoring the reliability and durability of the fuselage. The NASGRO equation, based on an erroneous hypothesis refuted by each farogram, is still used today. [See: Fracture Mechanics and Crack Growth Analysis Software SwRI NASGRO]. The NASGRO equation, based on an erroneous hypothesis refuted by each farogram, is still used today. [See: SwRI NASGRO Fracture Mechanics and Crack Growth Analysis Software]. However, nothing has changed in 35 years. In August 2022, a new version of equation 10.2 was proposed, based not on experiment but fictitious corrections.

It should be noted here that in 2014, there was a version of equation 3. Changing versions occurs by changing dimensionless coefficients without an experimental basis. Let's demonstrate the result of another experiment that refutes the NASGRO equation and similar ones.

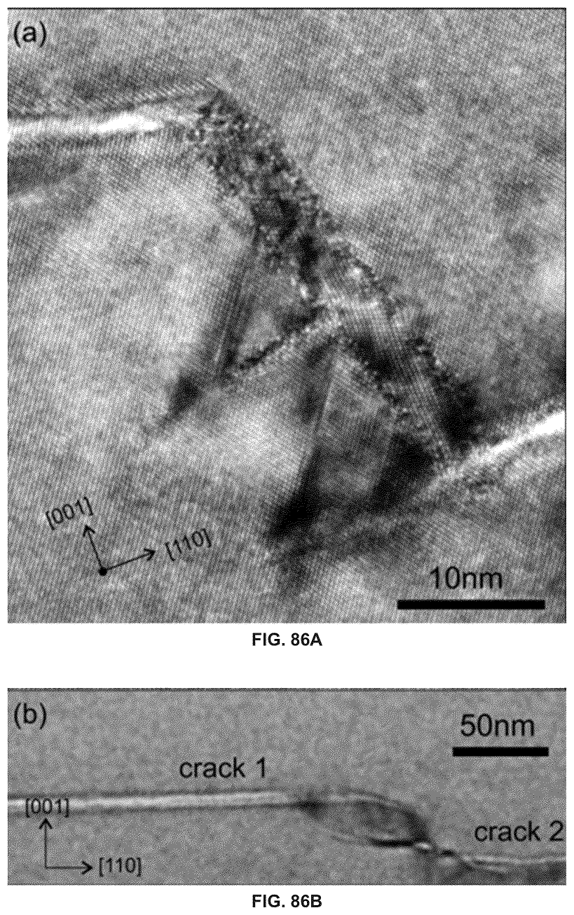

As shown in FIG. 86A and FIG. 86B, a single crack is formed, taken from the work [S. Reboh, J. F. Barbot, M. F. Beaufort, and P. F. P Fitchner: Propagation and interaction of cracks in Si induced by H supply into He filled cracks, J Appl. Phys. Lett. 96, 031907 2010].

In FIG. 86B, cracks 1 and 2 in the nanomaterial grow towards each other, as in the fuselage. Merging into one crack occurs, as shown in the photograph. Photo FIG. 86A, obtained at a higher resolution, allows us to conclude that the formation of a crack was preceded by a discharge, possibly a twofold one. Classical mechanics does not apply to nanomaterials; the electron microscope creates a photograph based on de Broglie waves. The use of the equation of mechanics to describe these processes is meaningless.

The identity of the processes of crack formation between rivets with those shown in FIG. 85 allows us to conclude that cracks originate at the nano level, and the mechanism of their initiation and growth cannot be described from the standpoint of classical mechanics, as is done in the four cited publications.

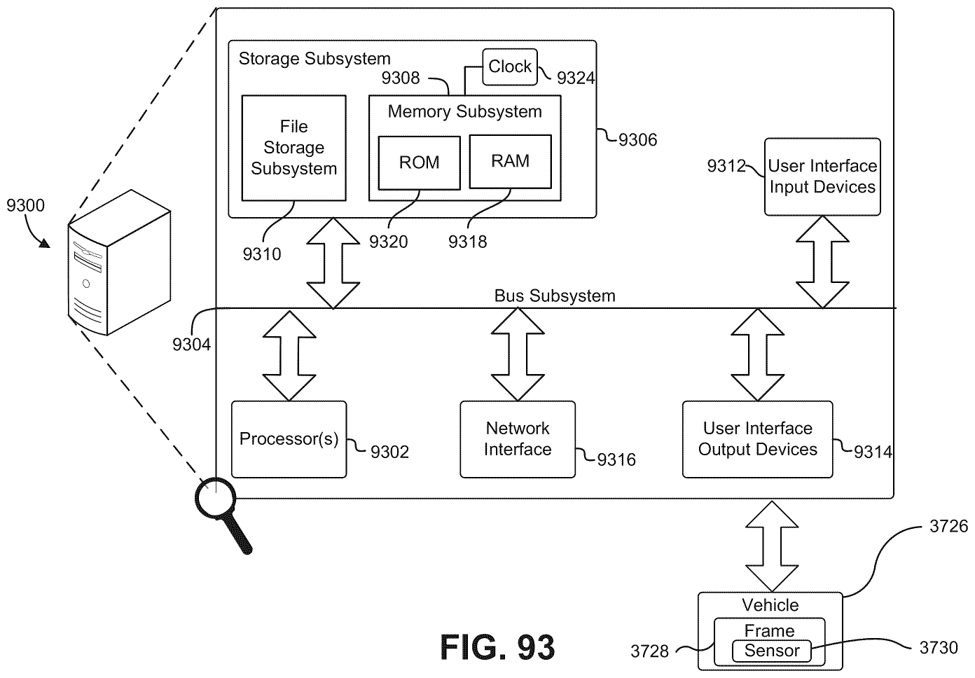

FIG. 93, is an illustrative, simplified block diagram of a computing device 9300 that can be used to practice at least one embodiment of the present disclosure. In at least one embodiment, computing device 9300 is used to implement any one or more aspects of the invention disclosed herein, including aspects related to detecting cracks, corrosion, pores, disease, viruses, glucose levels, or some combination thereof. In at least one embodiment, computing device 9300 is connected, wirelessly or otherwise, to a digital camera or sensing device configured to generate a farogram of an organic and/or inorganic object. The computing device 9300 causes instructions of software, installed in memory of the computing device, to be performed by a processor to obtain or otherwise receive the farogram in the form of data, such as bits. The software of the computing device 9300 analyzes the data of the farogram to identify aspects of the farogram that indicate various inorganic and/or organic processes occurring in the object, such as any of those described further herein. In at least one embodiment, computing device 9300 includes a neural network trained to infer semantic meaning, categories, classes, labels, or some combination thereof, of aspects identified in the farograms. A neural network can be any type or combination of types neural networks including convolutional neural networks, generative adversarial networks, recurrent neural networks, deep neural network, or perceptron neural networks.

In at least one embodiment, computing device 9300 is, or is a part of, a laptop, dedicated farogram generating system, a medical imaging device, desktop computer, data center, cloud computing system, or some combination thereof.

In various embodiments, the computing device 9300 may be used to implement any of the systems illustrated and described above. For example, the computing device 9300 may be configured for use as a data server, a web server, a portable computing device, a personal computer, or any electronic computing device. As shown in FIG. 93, the computing device 9300 may include one or more processors 9302 that, in embodiments, communicate with and are operatively coupled to a number of peripheral subsystems via a bus subsystem. In some embodiments, these peripheral subsystems include a storage subsystem 9306, comprising a memory subsystem 9308 and a file/disk storage subsystem 9310, one or more user interface input devices 9312, one or more user interface output devices 9314, and a network interface subsystem 9316. Such storage subsystem 9306 may be used for temporary or long-term storage of information.

In some embodiments, the bus subsystem 9304 may provide a mechanism for enabling the various components and subsystems of computing device 9300 to communicate with each other as intended. Although the bus subsystem 9304 is shown schematically as a single bus, alternative embodiments of the bus subsystem utilize multiple buses. The network interface subsystem 9316 may provide an interface to other computing devices and networks. The network interface subsystem 9316 may serve as an interface for receiving data from and transmitting data to other systems from the computing device 9300, such as sensor data, control signals (e.g., to control electromagnets affixed to a bridge), transmitting information (e.g., message indicating warnings about structural failure, and other examples). In some embodiments, the bus subsystem 9304 is utilized for communicating data locally and/or over a network.

In some embodiments, the user interface input devices 9312 includes one or more user input devices such as a keyboard; pointing devices such as an integrated mouse, trackball, touchpad, or graphics tablet; a scanner; a barcode scanner; a touch screen incorporated into the display; audio input devices such as voice recognition systems, microphones; and other types of input devices. In general, use of the term “input device” is intended to include all possible types of devices and mechanisms for inputting information to the computing device 9300. In some embodiments, the one or more user interface output devices 9314 include a display subsystem, a printer, or non-visual displays such as audio output devices, etc. In some embodiments, the display subsystem includes a cathode ray tube (CRT), a flat-panel device such as a liquid crystal display (LCD), light emitting diode (LED) display, or a projection or other display device. In general, use of the term “output device” is intended to include all possible types of devices and mechanisms for outputting information from the computing device 9300. The one or more user interface output devices 9314 can be used, for example, to present user interfaces to facilitate user interaction with applications performing processes described and variations therein, when such interaction may be appropriate. For example, a display interface may provide a graphical representation of a warning to an operator, a technician, or other employee to indicate results of measurements taken in accordance with embodiments described herein and conclusions derived therefrom.

In some embodiments, the storage subsystem 9306 provides a computer-readable storage medium for storing the basic programming and data constructs that provide the functionality of at least one embodiment of the present disclosure. The applications (programs, code modules, instructions), when executed by one or more processors in some embodiments, provide the functionality of one or more embodiments of the present disclosure and, in embodiments, are stored in the storage subsystem 9306. These application modules or instructions can be executed by the one or more processors 9302. In various embodiments, the storage subsystem 9306 additionally provides a repository for storing data used in accordance with the present disclosure. In some embodiments, the storage subsystem 9306 comprises a memory subsystem 9308 and a file/disk storage subsystem 9310.

In embodiments, the memory subsystem 9308 includes a number of memories, such as a main random access memory (RAM) 9318 for storage of instructions and data during program execution and/or a read only memory (ROM) 9320, in which fixed instructions can be stored. In some embodiments, the file/disk storage subsystem 9310 provides a non-transitory persistent (non-volatile) storage for program and data files and can include a hard disk drive, a floppy disk drive along with associated removable media, a Compact Disk Read Only Memory (CD-ROM) drive, an optical drive, removable media cartridges, or other like storage media. Memories of the computing device 9300 may be non-transitory and store instructions that are executable by one or more processors to cause the system to perform operations herein, such as applying logic to sensor data to infer conclusions to cause further operations (e.g., providing messages indicative of such conclusions, updating a graphical user interface, transmitting control signals to cause operation of another system (e.g., a brake subsystem, a warning alarm, and/or other such system). The logic can be in various forms, such as a rules engine, a decision tree, a neural network or other machine learning model, and/or other such computer-executable applications of logic to data.

In some embodiments, the computing device 9300 includes at least one local clock 9322. The at least one local clock 9322, in some embodiments, is a counter that represents the number of ticks that have transpired from a particular starting date and, in some embodiments, is located integrally within the computing device 9300. In various embodiments, the at least one local clock 9322 is used to synchronize data transfers in the processors for the computing device 9300 and the subsystems included therein at specific clock pulses and can be used to coordinate synchronous operations between the computing device 9300 and other systems in which the computing device is used. In another embodiment, the local clock is a programmable interval timer. In an embodiment, the computing device 9300 may communicate with a sensor 9330. In an embodiment, the sensor 9330 may be attached to a vehicle 9326. In another embodiment, the vehicle may possess a frame 9328 to which a sensor 9330 may be mounted.

The computing device 9300 could be of any of a variety of types, including a portable computer device, tablet computer, a workstation, or any other device described below. In at least one embodiment, computing device 9300 is a portable device taken into the field for a pipeline inspector to approach different sections of pipeline and inspect the structural integrity of the sections, with inspections discussed further in conjunction with at least FIGS. 3A-I. Additionally, the computing device 9300 can include another device that, in some embodiments, can be connected to the computing device 9300 through one or more ports (e.g., USB, a headphone jack, Lightning connector, etc.). In embodiments, such a device includes a port that accepts a fiber-optic connector. Accordingly, in some embodiments, this device is that converts optical signals to electrical signals that are transmitted through the port connecting the device to the computing device 9300 for processing. Due to the ever-changing nature of computers and networks, the description of the computing device 9300 depicted in FIG. 93 is intended only as a specific example for purposes of illustrating the preferred embodiment of the device. Many other configurations having more or fewer components than the system depicted in FIG. 93 are possible.

The proposed method allows you to abandon the methods, resulting in the product being destroyed or damaged so much that it is unsuitable for use.

The farogram of the process can be obtained at any stage of defect formation, as well as from any direction remotely, including for detecting damage to the inner surface by placing the sensor on the outer one. The process can be obtained in a fraction of a second, and its objectivity does not require proof. The farogram is created by photons emitted due to an atomic-proton reaction due to a natural phenomenon that does not require a source of energy created by man.

The proposed method makes it possible to monitor the manufacturing quality of the connection of fuselage elements during wear and operation remotely, informing the crew. Farogram of the process can be obtained in a fraction of a second, and its objectivity does not require proof. The farogram is created by photons emitted due to an atomic-proton reaction due to a natural phenomenon that does not require a source of energy created by man.

The proposed method makes it possible to monitor the manufacturing quality of the connection of fuselage elements during wear and operation remotely, informing the crew.

The problem of monitoring changes in the state of the pipeline due to mechanical, chemical, and radiation exposure remains relevant.

There are no modern, non-destructive methods of continuous monitoring of the inner surface of the pipeline. Using p-radiation makes it possible to control the process of nucleation and development of corrosion and to crack based on the difference in the frequency of photons mitted under mechanical, chemical, and radiation.

The farograms obtained during the study of pipes under mechanical and chemical influences and multi-colored areas indicate different frequencies of the emitted photons by various metals and non-metals.

This means that material described in U.S. Pat. No. 11,047,813 B2 can be used to monitor pipeline damage and predict to prevent catastrophic failure.

The pipeline investigation under mechanical and chemical action confirmed the possibility of using the self-emission transparency of p-radiation.

The use of the method proposed in this invention implements this law.

Farograms do not exclude the possibility of using other control methods. On the contrary, they can complement each other.

The main controlled parameters are the pipeline wall thickness and the rate of its decrease. The pipeline walls' thickness reduction occurs due to damage to the outer and inner surfaces resulting from corrosion and cracking. A distinctive feature of this invention lies in that damage to the inner surface is controlled from the outer surface using the self-emission transparency of p-radiation. However, this does not exclude the possibility of using p-radiation inside the pipeline when access to the outer surface is impossible. The device called a smart pig, is supplemented with p-radiation sensors. This method for assessing the thickness of the material, used for the first time, requires experimental and theoretical justification.

Previously, the corrosion layer's thickness was investigated with direct access to it repeatedly using ultrasound and electromagnetic methods. For example, X-ray photoelectron spectroscopy was used in [F. M. Al-Kharafi et al. in Industrial Corrosion and Corrosion Control Proceedings of the 2nd Arabian Corrosion Conference Kuwait, (1996), 416-429], which investigated the effect of chloride ion concentration on the corrosion and passivation behavior of Al, Al—Cu, Al-6061, and Al-70753. The energies of Al ions were determined: at 2p −75.5 eV, 2s −120 eV; Cu at 2p³-932.5 eV and Cu at 2p¹-952.5 eV; C at is −285.5 eV; O at1s 532.5 eV.

The pipeline walls' thickness reduction occurs due to damage to the outer and inner surfaces resulting from corrosion and cracking. A distinctive feature of this invention lies in that damage to the inner surface is controlled from the outer surface using the self-emission transparency of p-radiation. However, this does not exclude the possibility of using p-radiation inside the pipeline when access to the outer surface is impossible. The device called a smart pig, is supplemented with p-radiation sensors.

Analysis of farograms allows us to conclude that the effect of a constant magnetic field on atomic fusion reactions and atomic reactions caused by deformation is different, just as the mechanism is different. For example, the magnetic field inhibits the synthesis of water and compounds, slowing the corrosion process. In contrast, deformation under the influence of a magnetic field decreases the energy density of the electromagnetic pulse, at which cracks are not formed. Still, the energy of the emitted photons (their frequency) increases, and the absorbed photons decrease. The inventor experimented, and its interpretation indicates how the fabrication must be carried out to protect the products from damage. Of particular importance are the magnitude of the induction of the magnetic field, the direction of the magnetic field, etc.

The development of hydrogen power engineering requires effective methods to control the conditions of hydrogen storage and its use. The use of farograms for these purposes is the most promising.

Lasers are widely used in engineering, biology, and medicine. For example, using p-radiation caused by lasers significantly expands the possibilities and quality of control of processes in the cells of plants, organs, multi-ton products, water bodies, and the earth's surface cells, of plants, organs, multi-ton products, water bodies, and the earth's surface.

### Safe Synthesis of High-Toxic Compounds

Thallium and some of its compounds are especially dangerous. The solid-phase synthesis of compounds containing thallium presents extreme difficulties associated with the possibility of unexpected depressurization of the ampoule in which it is carried out. This leads to the synthesis being carried out in small quantities for a long time. The inventor synthesized the compound to create a switching and memory device on a single crystal but not a chalcogenide glass. The attempt turned out to be successfully obtained a 1×1×1 mm³ single crystal was, discovered the effect was the published article [See, L. N. Antipova, B. V. Beliaev, V. A. Gorkov, V. P. Rombakh, Switching Effect on Crystalline, Physics, and Technics of Semiconductors (1972) Vol. 5, No. 12, 2397, 2398]. The article was used as a prototype of U.S. Pat. No. 3,991,330. The authors patented the high piezoelectric effect on this crystalline compound, which is particularly interesting for acousto-electronics. However, it was impossible to provide the required quantity and quality of single crystals for creating devices using solid-phase synthesis methods. The inventor developed and used an explosion-proof method to synthesize a compound consisting of two stages, which made it possible to synthesize 6 kg of the compound in one ampoule for three days. The required quality of the compound was ensured by additional purification of extra-pure starting components from carbon, which, according to M. Faraday's experiments, got into it from the carbon dioxide in the air. The validity of M. Faraday's conclusion is confirmed by the fact that the capillary walls darkened after the purification of ultra-pure sulfur by dripping through a capillary.

The quality of the single crystal and its dimensions were ensured by growing in an argon atmosphere using sevenfold zone recrystallization. It assumes that this excluded the synthesis of water.

This made it possible to synthesize other class compounds, which were

B=V, Nb, Ta; C=S, Se, Te. The Tl—Ta—Te system absorbs X-rays intensively.

This example is given to show one of the problems of cracking in synthesis.

The inventor and his employees, V. A. Gritskikh and V. A. Korotun, synthesized the compound. And grew from it single crystals 3×3×1 mm³ of a multicomponent high-temperature superconductor.

$${Tl}_{2 - x} {Bi}_{x} {Ba}_{2 - y} {Pb}_{y} {Ca}_{2} {Cu}_{3} {CuO}_{{10}_{-} + 8}$$

with a transition temperature of 123 K.

### Hot Cracks in the Synthesis of Multicomponent Alloys

The number of alloys containing from two to seven metals is practically unlimited.

Modern scientific journals publish the results of the synthesis and study of the properties of dozens of new alloys annually. A common challenge for the new alloy remains the problem of cracks and corrosion cracking. It is generally accepted that the main reason for the formation of hot cracks is in the stress-strain state. This builds confidence that one can solve the problem using mechanics. But in nature, there is no mechanical interaction between atoms. Newton's force loses its meaning at a distance of fewer than four nanometers since the wave properties of the atom are manifested.

An analysis of seven thousand publications on strength and fracture problems, carried out by the inventor over twenty-two years, showed that none contains the word photon—the chemical reaction characterizing corrosion recorded without his participation.

Eleven employees of scientific laboratories in Germany and France carried out a study of the formation of an alloy of seven chemical elements Ni—Co—Cr—Mo—Al—Ti—B, using melting in a vacuum or argon atmosphere using an electron beam or laser to describe the mechanism of hot cracking. The study of the processes was carried out using modern equipment [See, E. Chauvet et al. Hot cracking mechanism affecting a non-weldable Ni-based superalloy produced by selective electron Beam Melting, Acta Materialia, (2018), 142 82-94]. Both mechanisms of crack formation, proposed by the authors, do not consider the electromagnetic nature of the formation of pores, cracks, the liquid they discovered, and the synthesis of boron (B) compounds with metal (M): MB, M₂B, and M₅B₃. Metal is not specified.

Lithium, beryllium, and boron are particularly interesting for creating alloys. The atoms of these metals have the lowest nuclear charge: 3, 4, 5. Obtaining the programs of alloys containing these metals is due to the lowest charge of the atomic nucleus (3, 4, 5). The electronic configuration of the lithium atom is 1s²2s¹. A chemical reaction involving lithium will occur during ionization, which requires the appropriate photon energy. The first ionization potential of lithium is I₁=5.39 eV; the second is I₂=75.64 eV, and the third is I₃=122.45 eV. Excitation of electrons from the is shell is accompanied by emission of X-rays. The inventor recorded p-radiation from a working lithium battery. Excitation of X-rays with an energy of 122.45 eV is possible only when an electrostatic field accelerates electrons, the potential difference which significantly exceeds this value.

The inventor recorded p-radiation from a lithium battery at a voltage of 12 V through a polypropylene case. This experiment confirms the inventor's hypothesis that p-radiation is due to protons' excitation of an electron.

The ionization potentials of electrons from the is shell of beryllium and boron are higher than those of lithium. Consequently, the synthesis of compounds with these elements can be monitored using a farogram.

Farograms for controlling the synthesis of compounds allow you to create alloys with desired properties. Farograms allow technologists to select the alloy, ensuring the product's maximum safety during the entire intended service life. Farograms obtained during the operation of the product make it possible to stop using it before the onset of a critical state.

The examples show that the spectral lines caused by the excitation of valence electrons, which characterize a chemical element, accompany the p-radiation caused by the impact of protons. These two processes are conditioned by the law, which the inventor formulates. The experiment shows that the probability of excitation of an atom by a proton exceeds that of excitation by an electron and a photon. The repulsion of a proton by the atomic nucleus prevents the formation of p-radiation. However, the fact that it is observed in the lead indicates that the photon excites the electron, not from the atom's valence shell but from the nearest shell under it. For example, five electrons are distributed over the shells in boron 1s²2s²p¹.

Boron exhibits a maximum valence of three. Consequently, p-radiation is due to the excitation of an electron on the shell 1s.

The existence of numerous compounds of boron with hydrogen, up to BₙHₙ²⁻ (n=10, 12) [See: E. A. Malinina, Clusters of Boron Anions (n=10, 12) as Ligand in Coordination Compounds of Metal IB Group and Lead (II), Abstract of Doctoral dissertation, Moscow, 2009, 46 pages (in Russian).] indicates that two electrons are removed from two hydrogen atoms. But this can also be explained by the fact that two additional protons are absorbed.

The analysis of such processes is of great importance for hydrogen energy and storage, but studying the processes is possible only with the help of farograms.

This led to the broad possibilities of the practical application of p-radiation, some of which are given in the present invention.

Thus, one more experiment shows that the nature of cracking and its mechanism can only be described from the standpoint of quantum mechanics.

Today, scientific laboratories in highly developed countries use the most high-precision, susceptible equipment for studying the properties of materials created thanks to the success of quantum mechanics. Often, research is carried out by teams from several laboratories, even from different countries. The photographs obtained using X-ray diffraction, neutron diffraction, and electron backscattering are black and white. Still, the authors paint the areas in different colors to describe the processes caused by the influence of particular chemical elements. However, the color proposed by the authors does not carry any information about the synthesis, destruction, or reduction processes, such as iron from compounds using hydrogen. When starting a research, the authors often declare that the mechanism of the phenomenon is unclear or unknown. Still, they try to explain it in the works based on the theory refuted by the experiment.

The research carried out by the inventor and the law proposed by him allow us to describe the mechanism of fracturing and synthesis.

Let's call a photograph, graph, or a set of numbers obtained from the research a farogram. Thus, the first step in the application of the method proposed by the invention is to create a device for creating a farogram, including measurements of the intensity of p-radiation, its frequency, the transmission of information, its storage and use by the object, the safe functioning of which it should provide.

The advantage of using farograms is due to:

1. Using the electromagnetic spectrum, which is the only source of information about the energy state of atoms, guarantees the results' reliability and objectivity.

2. The versatility allows the method to be used in any field of human activity at any object.

3. High penetrating power allows you to explore large-sized multi-ton structures to investigate devices inaccessible for direct contact remotely.

4. A short wavelength that allows viruses to be detected.

5. Maximum possible signal transmission speed of measurement results at any distance, including via satellite.

6. High efficiency and low cost.

7. There are two types of X-radiation: bremsstrahlung (continuous) and characteristic. This name is because characteristic radiation is observed in the form of a narrow spectral line characteristic of an atom of a chemical element. Such lines are observed when an electron moves away from orbits near the nucleus. The scattering of charged particles in the electric field of an atomic nucleus causes Bremsstrahlung. The magnetic field changes the trajectory of a charged particle. The farogram illustrates this fact, which predetermines the possibility of using a magnetic field to inhibit an atomic reaction. The farogram illustrates lines and solid-colored areas. This means that the solid-colored regions of the p-radiation should be regarded as characteristic. Consequently, the farogram expands the possibilities of spectral analysis.

A farogram can be obtained in any experimental study. The photographic method does not exclude the possibility of detecting electromagnetic signals with the help of sensors and developing computer programs for analyzing the processes of initiation and development of damage in various objects and/or substances. The inventor publishes books and an article to implement the method proposed in the invention.

V. P. Rombakh, Non-Invasive Use of Electromagnetic Radiation Caused by Atomic-Proton and Proton-Electron Interaction in all Field of Human Activity (2022) 210 pages.

2. V. P. Rombakh, P-radiation of Atomic Reaction and its use, (2021) 120 pages.

3. V. P. Rombakh. Non-Invasive Monitoring of Atomic Reaction and its Applications, (2019), 187 pages.

4. V. P. Rombakh, Two Interpretations of Maxwell's Mechanics, (2018) 87 pages.

5. V. P. Rombakh, Introduction to the Physics of Destruction (2014) 196 pages.

6. V. P. Rombakh, Why the Volgograd Bridge didn't collapse and how to avoid a catastrophe, (2011) Vestnik Volgu. Series 10, Issue 5, 72-80 pages

7. V. P. Rombakh, Atom Parameters, and Metal Properties. Logistics Capital Inc., Edmonds, WA 2008, P. 313.

8. V. P. Rombakh, Introduction to Fracture Physics, Edmonds, Logistics Capital Inc., Edmonds, WA 2008 WA USA 2014, p. 297.

9. V. P. Rombakh, The electronic mechanism for the formation of cracks in metal,

10. V. P. Rombakh, Maxwell's theory of elasticity, the idea of the potential energy of distortion and its quantum mechanical interpretation, Belgorod: Publishing house of BSTU, (2015) 185-197.

11. V. P. Rombakh, Physical foundations of materials science, Saransk: Mordov Publishing House. (2016) 46-57.

12. V. P. Rombakh, Reliability, and durability of structures and devices, Collection of materials of the IV International scientific and practical conference, Kemerovo, (2016), vol. II, 205-209.

13. V. P. Rombakh, Nondestructive Monitoring of Atomic Reactions to Detect Precursors of Structural Failure, Singapore, WAST. org, Manuscript ID: 158487-Review Report (in press).

At least one embodiment of the disclosure can be described in view of the following clauses:
1. A method, comprising:
using a sensor to detect p-photon radiation activity within an object; and
locating damage within the object based, at least in part, on the sensor detecting the p-photon radiation activity.
2. The method of clause 1, wherein a characteristic of the damage is indicated by an image generated based, at least in part, on the p-photon radiation activity detected by the sensor.
3. The method of any of clauses 1-2, wherein:
a characteristic of the damage is identified by one or more frequencies of one or more p-photons; and
the one or more frequencies are identified by one or more colors in an image generated based, at least in part, on the sensor detecting the p-photon radiation activity.
4. The method of any of clauses 1-3, wherein:
a characteristic of the damage is identified by one or more shapes in an image; and
the image is generated based, at least in part, on the sensor detecting the p-photon radiation activity.
5. The method of any of clauses 1-4, wherein one or more indications of water synthesis are generated based, at least in part, on the sensor detecting the p-photon radiation activity.
6. The method of any of clauses 1-5, wherein the method further comprises positioning a screen through which p-photons pass before reaching the sensor.
7. The method of any of clauses 1-6, wherein the damage is associated with pathologies of human organs.
8. A system, comprising:
one or more processors; and
memory including executable instructions that, if executed by the one or more processors, cause the system to:
   use a sensor to detect p-photon radiation activity within an object; and
   locate damage within the object based, at least in part, on the sensor detecting the p-photon radiation activity.
9. The system of clause 8, wherein a characteristic of the damage is indicated by an image generated based, at least in part, on the p-photon radiation activity detected by the sensor.
10. The system of any of clauses 8-9, wherein:
a characteristic of the damage is identified by one or more frequencies of one or more p-photons; and
the one or more frequencies are identified by one or more colors in an image generated based, at least in part, on the sensor detecting the p-photon radiation activity.
11. The system of any of clauses 8-10, wherein:
a characteristic of the damage is identified by one or more shapes in an image; and
the image is generated based, at least in part, on the sensor detecting the p-photon radiation activity
12. The system of any of clauses 8-11, wherein one or more indications of water synthesis are generated based, at least in part, on the sensor detecting the p-photon radiation activity.
13. The system of any of clauses 8-12, wherein the method further comprises positioning a screen through which p-photons pass before reaching the sensor.
14. The system of any of clauses 8-13, wherein the damage is associated with pathologies of human organs.
15. A non-transitory computer-readable storage medium having stored thereon executable instructions that, if executed by one or more processors of a computer system, cause the computer system to at least:
   use a sensor to detect p-photon radiation activity within an object; and
   locate damage within the object based, at least in part, on the sensor detecting the p-photon radiation activity.
16. The non-transitory computer-readable storage medium of clause 15, wherein a characteristic of the damage is indicated by an image generated based, at least in part, on the p-photon radiation activity detected by the sensor.
17. The non-transitory computer-readable storage medium of any of clauses 15-16, wherein:
a characteristic of the damage is identified by one or more frequencies of one or more p-photons; and
the one or more frequencies are identified by one or more colors in an image generated based, at least in part, on the sensor detecting the p-photon radiation activity.
18. The non-transitory computer-readable storage medium of any of clauses 15-17, wherein:
a characteristic of the damage is identified by one or more shapes in an image; and
the image is generated based, at least in part, on the sensor detecting the p-photon radiation activity.
19. The non-transitory computer-readable storage medium of any of clauses 15-18, wherein one or more indications of water synthesis are generated based, at least in part, on the sensor detecting the p-photon radiation activity.
20. The non-transitory computer-readable storage medium of any of clauses 15-19, wherein the method further comprises positioning a screen through which p-photons pass before reaching the sensor.

Other variations are within spirit of present disclosure. Thus, while disclosed techniques are susceptible to various modifications and alternative constructions, certain illustrated embodiments thereof are shown in drawings and have been described above in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of this disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

Numerous specific details are set forth to provide a more thorough understanding of at least one embodiment. However, it will be apparent to a skilled person that these inventive concepts may be practiced without one or more of these specific details and that aspects of one or more embodiments described herein can be combined.

What is claimed is:

1. A method, comprising:
using a sensor to detect p-photon radiation activity within an object; and
locating damage within the object based, at least in part, on the sensor detecting the p-photon radiation activity.

2. The method according to claim 1, wherein a characteristic of the damage is indicated by an image generated based, at least in part, on the p-photon radiation activity detected by the sensor.

3. The method according to claim 1, wherein:
a characteristic of the damage is identified by one or more frequencies of one or more p-photons; and
the one or more frequencies are identified by one or more colors in an image generated based, at least in part, on the sensor detecting the p-photon radiation activity.

4. The method according to claim 1, wherein:
a characteristic of the damage is identified by one or more shapes in an image; and
the image is generated based, at least in part, on the sensor detecting the p-photon radiation activity.

5. The method according to claim 1, wherein one or more indications of water synthesis are generated based, at least in part, on the sensor detecting the p-photon radiation activity.

6. The method according to claim 1, wherein the method further comprises positioning a screen through which p-photons pass before reaching the sensor.

7. The method according to claim 1, wherein the damage is associated with pathologies of human organs.

8. A system, comprising:
one or more processors; and
memory including executable instructions that, if executed by the one or more processors, cause the system to:
use a sensor to detect p-photon radiation activity within an object; and
locate damage within the object based, at least in part, on the sensor detecting the p-photon radiation activity.

9. The system of claim 8, wherein a characteristic of the damage is indicated by an image generated based, at least in part, on the p-photon radiation activity detected by the sensor.

10. The system of claim 8, wherein:
a characteristic of the damage is identified by one or more frequencies of one or more p-photons; and
the one or more frequencies are identified by one or more colors in an image generated based, at least in part, on the sensor detecting the p-photon radiation activity.

11. The system according to claim 8, wherein:
a characteristic of the damage is identified by one or more shapes in an image; and
the image is generated based, at least in part, on the sensor detecting the p-photon radiation activity

12. The system of claim 8, wherein one or more indications of water synthesis are generated based, at least in part, on the sensor detecting the p-photon radiation activity.

13. The system of claim 8, wherein the method further comprises positioning a screen through which p-photons pass before reaching the sensor.

14. The system of claim 8, wherein the damage is associated with pathologies of human organs.

15. A non-transitory computer-readable storage medium having stored thereon executable instructions that, if executed by one or more processors of a computer system, cause the computer system to at least:
use a sensor to detect p-photon radiation activity within an object; and
locate damage within the object based, at least in part, on the sensor detecting the p-photon radiation activity.

16. The non-transitory computer-readable storage medium of claim 15, wherein a characteristic of the damage is indicated by an image generated based, at least in part, on the p-photon radiation activity detected by the sensor.

17. The non-transitory computer-readable storage medium of claim 15, wherein:
a characteristic of the damage is identified by one or more frequencies of one or more p-photons; and
the one or more frequencies are identified by one or more colors in an image generated based, at least in part, on the sensor detecting the p-photon radiation activity.

18. The non-transitory computer-readable storage medium of claim 15, wherein:
a characteristic of the damage is identified by one or more shapes in an image; and
the image is generated based, at least in part, on the sensor detecting the p-photon radiation activity.

19. The non-transitory computer-readable storage medium of claim 15, wherein one or more indications of water synthesis are generated based, at least in part, on the sensor detecting the p-photon radiation activity.

20. The non-transitory computer-readable storage medium of claim 15, wherein the method further comprises positioning a screen through which p-photons pass before reaching the sensor.
